SEQUENCE LISTING (40 sequences)

SEQ ID NO: 1 - protein, 5 aa, synthetic construct
SNAMH

SEQ ID NO: 2 - protein, 17 aa, synthetic construct
  VARIANT 6: note = N or A
  VARIANT 7: note = G or A
WIKAGXXEIKYSQKFQG

SEQ ID NO: 3 - protein, 10 aa, synthetic construct
GDYYYYYMDV

SEQ ID NO: 4 - protein, 11 aa, synthetic construct
RASQSISDFLN

SEQ ID NO: 5 - protein, 7 aa, synthetic construct
  VARIANT 7: note = N or A
AASSLQX

SEQ ID NO: 6 - protein, 9 aa, synthetic construct
QQSYSTPLS

SEQ ID NO: 7 - protein, 119 aa, synthetic construct
  VARIANT 55: note = N or A
  VARIANT 56: note = G or A
QVQLVQSGAEVKKPGASVKVSCKASGYTFTSNAMHWVRQVPGQGLEWMGWIKAGXXEIKYSQKFQGRVTITRDTSATTAYMELSSLRSEDTAVYYCARGDYYYYYMDVWGKGTTVTVSS

SEQ ID NO: 8 - protein, 107 aa, synthetic construct
  VARIANT 56: note = N or A
DIQVTQSPSSLSASVGDRVTITCRASQSISDFLNWYQQKPGKAPKLLIHAASSLQXGVPSRFSGSGSGTDFSLTINSLQPEDFATYYCQQSYSTPLSFGGGTKVEIK

SEQ ID NO: 9 - protein, 5 aa, synthetic construct
  VARIANT 2: note = Y or F
SXAMQ

SEQ ID NO: 10 - protein, 17 aa, synthetic construct
  VARIANT 6: note = N or A
  VARIANT 7: note = G or A
WIIAGXXDTKYSQNFQD

SEQ ID NO: 11 - protein, 12 aa, synthetic construct
GSGFSGYGYFDK

SEQ ID NO: 12 - protein, 11 aa, synthetic construct
RASHSISSYLN

SEQ ID NO: 13 - protein, 7 aa, synthetic construct
ASSSLQS

SEQ ID NO: 14 - protein, 9 aa, synthetic construct
QQSHSIPLT

SEQ ID NO: 15 - protein, 121 aa, synthetic construct
  VARIANT 55: note = N or A
  VARIANT 56: note = G or A
QVQLVQSGAEVKKPGASVKVSCKASGYTFTSYAMQWVRQAPGQRLEWMGWIIAGXXDTKYSQNFQDRVTLTRDTSANTAYMDLSGLRSEDTAVYYCARGSGFSGYGYFDKWGQGTLVTVSS

SEQ ID NO: 16 - protein, 107 aa, synthetic construct
  VARIANT 65: note = N or S
DIQMTQSPSSLSASVGDRVTITCRASHSISSYLNWYQQKPGKVPKLLIYASSSLQSGVPSRFSGXGSGTDFTLIISSLQPEDFATYYCQQSHSIPLTFGGGTKVEIK

SEQ ID NO: 17 - protein, 121 aa, synthetic construct
QVQLVQSGAEVKKPGASVKVSCKASGYTFTSFAMQWVRRAPGQRLEWMGWIIAGNGDTKYSQNFQDRVTLTRDTSANTAYMDLSGLRSEDTAVYYCARGSGFSGYGYFDKWGQGTLVTVSS

SEQ ID NO: 18 - protein, 107 aa, synthetic construct
DIQMTQSPSSLSASVGDRVTITCRASHSISSYLNWYQQKPGKVPKLLIYASSSLQSGVPSRFSGNGSGTDFTLIISSLQPEDFATYYCQQSHSIPLTFGGGTKVEIK

SEQ ID NO: 19 - protein, 326 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 20 - protein, 107 aa, synthetic construct [sequence omitted]

SEQ ID NO: 21 - protein, 602 aa, synthetic construct
MEPAGPAPGRLGPLLCLLLAASCAWSGVAGEEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSAHPKEQGSNTAAENTGSNEREPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 22 - protein, 380 aa, synthetic construct
MEPAGPAPGRLGPLLCLLLAASCAWSGVAGEEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSAHPKEQGSNTAAENTGSNERHHHHHHHHHH

SEQ ID NO: 23 - protein, 374 aa, synthetic construct
MDMRVPAQLLGLLLLWFPGSRCEDELQVIQPEKSVSVAAGESATLRCAMTSLIPVGPIMWFRGAGAGRELIYNQKEGHFPRVTTVSELTKRNNLDFSISISNITPADAGTYYCVKFRKGSPDDVEFKSGAGTELSVRAKPSAPVVSGPAVRATPEHTVSFTCESHGFSPRDITLKWFKNGNELSDFQTNVDPAGDSVSYSIHSTARVVLTRGDVHSQVICEIAHITLQGDPLRGTANLSEAIRVPPTLEVTQQPMRAENQANVTCQVSNFYPRGLQLTWLENGNVSRTETASTLIENKDGTYNWMSWLLVNTCAHRDDVVLTCQVEHDGQQAVSKSYALEISAHQKEHGSDITHEAALAPTAPLHHHHHHHHHH

SEQ ID NO: 24 - protein, 370 aa, synthetic construct
MPVPASWPHPPGPFLLLTLLLGLTEVAGEEELQMIQPEKLLLVTVGKTATLHCTVTSLLPVGPVLWFRGVGPGRELIYNQKEGHFPRVTTVSDLTKRNNMDFSIRISSITPADVGTYYCVKFRKGSPENVEFKSGPGTEMALGAKPSAPVVLGPAARTTPEHTVSFTCESHGFSPRDITLKWFKNGNELSDFQTNVDPTGQSVAYSIRSTARVVLDPWDVRSQVICEVAHVTLQGDPLRGTANLSEAIRVPPTLEVTQQPMRVGNQVNVTCQVRKFYPQSLQLTWSENGNVCQRETASTLTENKDGTYNWTSWFLVNISDQRDDVVLTCQVKHDGQLAVSKRLALEVTVHQKDQSSDATPGPASHHHHHH

SEQ ID NO: 25 - protein, 379 aa, synthetic construct
MEPAGPAPGRLGPLLCLLLTASCAWSGVLGEEELQVIQPEKSVSVAAGDSATLNCTVSSLIPVGPIQWFRGAGPGRELIYNLKEGHFPRVTAVSDPTKRNNMDFSIRISNITPADAGTYYCVKFRKGSPDVELKSGAGTELSVRAKPSAPVVSGPAVRATAEHTVSFTCESHGFSPRDITLKWFKNGNELSDVQTNVDPAGKSVSYSIRSTARVLLTRRDVHSQVICEVAHVTLQGDPLRGTANLSEAIRVPPFLEVTQQSMRADNQVNVTCQVTKFYPQRLQLTWLENGNVSRTEMASALPENKDGTYNWTSWLLVNVSAHRDDVKLTCQVEHDGQPAVNKSFSVKVSAHPKEQGSNTAAENTGTNERHHHHHHHHHH

SEQ ID NO: 26 - protein, 513 aa, Homo sapiens
MEPAGPAPGRLGPLLCLLLAASCAWSGVAGEEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSAHPKEQGSNTAAENTGSNERNIYIVVGVVCTLLVALLMAALYLVRIRQKKAQGSTSSTRLHEPEKNAREITQDTNDITYADLNLPKGKKPAPQAAEPNNHTEYASIQTSPQPASEDTLTYADLDMVHLNRTPKQPAPKPEPSFSEYASVQVPRKYPYDVPDYA

SEQ ID NO: 27 - protein, 305 aa, Homo sapiens
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRNN

SEQ ID NO: 28 - protein, 368 aa, synthetic construct
MGWSCIILFLVATATGVHSEEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSAHPKEQGSNTAAENTGSNERHHHHHHHHHH

SEQ ID NO: 29 - protein, 372 aa, synthetic construct
MGWSCIILFLVATATGVHSGVAGEEELQVIQPDKSVLVAAGETATLRCTATSLIPVGPIQWFRGAGPARELIYNQKEGHFPRVTTVSESTKRENMDFSISISNITPADAGTYYCVKFRKGSPDTEFKSGAGTELSVRAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSAHPKEQGSNTAAENTGSNERHHHHHHHHHH

SEQ ID NO: 30 - protein, 153 aa, synthetic construct
MDMRVPAQLLGLLLLWFPGSRCQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPHHHHHHHHHH

SEQ ID NO: 31 - protein, 353 aa, synthetic construct
QLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 32 - protein, 440 aa, synthetic construct
EVQLQQSGAELVKPGASVKLSCTASGFNIKDYYIHWVQQRTEQGLEWIGRIDPEDGETKYAPKFQDKATITADTSSNTAYLHLSSLTSEDTAVYYCARWGAYWGQGTLVTVSAASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 33 - protein, 215 aa, synthetic construct
QIVLTQSPAIMSASPGEKVTLTCSASSSVSSSYLYWYQQKPGSSPKLWIYSTSNLASGVPARFSGSGSGTSYSLTISSMEAEDAASYFCHQWSSYPRTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 34 - protein, 447 aa, synthetic construct
EVQLVQSGAEVKKPGESLRISCKASGYSFTSYWVHWVRQMPGKGLEWMGNIDPSDSDTHYSPSFQGHVTLSVDKSISTAYLQLSSLKASDTAMYYCVRGGTGTLAYFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 35 - protein, 219 aa, synthetic construct
DVVMTQSPLSLPVTLGQPASISCRSSQSLVHSYGNTYLYWFQQRPGQSPRLLIYRVSNRFSGVPDRFSGSGSGTDFTLKISRVEAEDVGVYYCFQGTHVPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 36 - protein, 602 aa, synthetic construct
MGADDVVDSSKSFVMENFSSYHGTKPGYVDSIQKGIQKPKSGTQGNYDDDWKGFYSTDNKYDAAGYSVDNENPLSGKAGGVVKVTYPGLTKVLALKVDNAETIKKELGLSLTEPLMEQVGTEEFIKRFGDGASRVVLSLPFAEGSSSVEYINNWEQAKALSVELEINFETRGKRGQDAMYEYMAQACAGNRVRRSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKHIDEILAALPKTDTYKLILNGKTLKGETTTEAVDAATAEKVFKQYANDNGVDGEWTYDDATKTFTVTEKPEVIDASELTPAVTTYKLVINGKTLKGETTTEAVDAATAEKVFKQYANDNGVDGEWTYDDATKTFTVTEKPEVIDASELTPAVTTYKLVINGKTLKGETTTKAVDAETAEKAFKQYANDNGVDGVWTYDDATKTFTVTELEHHHHHH

SEQ ID NO: 37 - protein, 373 aa, synthetic construct
MEPAGPAPGRLGPLLLCLLLSASCFCTGATGTEVKVTQPEKSVSVAAGDSTILNCTVTSLLPVGPIRWYRGVGQSRLLIYSFTGEHFPRVRNVSDTTKRNNMDFSIRISNVTPEDAGTYYCVKFQRGSSEPDTEIQSGGGTEVYVLAKPSPPEVSGPADRGIPDQKVNFTCKSHGFSPRNITLKWFKDGQELHPLETTVNPSGKNVSYNISSTVRVVLNSMDVNSKVICEVAHITLDRSPLRGIANLSNFIRVSPTVKVTQQPPTSMNQVNLTCRAERFYPEDLQLIWLENGNVSRNDTPKNLTKNTDGTYNYTSLFLVNSSAHREDVVFTCQVKHDQQPAITRNHTVLGLAHSSDQGSMQTFHHHHHHHHHH

SEQ ID NO: 38 - protein, 329 aa, synthetic construct
  VARIANT 135: note = M or Y
  VARIANT 137: note = S or T
  VARIANT 139: note = T or E
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAQGGPSVFLFPPKPKDTLXIXRXPEVTCVVVDVSHEDPEVKFNWYVDGVEVHNAKTKPREEQYNSTYRVVSVLTVLHQDWLNGKEYKCQVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 39 - protein, 448 aa, synthetic construct
QVQLVQSGAEVKKPGASVKVSCKASGYSFTNYWVNWVRQAPGQGLEWIGMVHPSGSETRLNQQFKDKATLTVDKSTSTAYMELSSLRSEDTAVYYCARVWSGNAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 40 - protein, 220 aa, synthetic construct
DIVMTQSPDSLAVSLGERATINCLSSQSLLASGNQRNYLTWYQQKPGQPPKLLIYWASTRDSGVPDRFTGSGSGTDFTLTISSVQAEDVAVYYCQNGFSFPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2025/0250355 A1
(43) Pub. Date: Aug. 7, 2025

(54) ANTIBODIES TARGETING SIRP-ALPHA AND USES THEREOF

(71) Applicant: BIOSION INC., Nanjing, Jiangsu (CN)

(72) Inventors: Mingjiu CHEN, Nanjing (CN); Mark Zhiqing MA, Nanjing (CN); Zeyu PENG, Nanjing (CN); Jinyu LIU, Nanjing (CN)

(21) Appl. No.: 18/857,004

(22) PCT Filed: Apr. 20, 2023
(86) PCT No.: PCT/CN2023/089551

§ 371 Date: Oct. 15, 2024

(30) Foreign Application Priority Data
Apr. 20, 2022 (WO) PCT/CN2022/088000

(51) Int. Cl. C07K 16/28; A61K 39/00; A61P 37/04

(52) U.S. Cl. CPC C07K 16/2896; A61P 37/04; A61K 2039/505; C07K 2317/33; C07K 2317/565; C07K 2317/77; C07K 2317/92

(57) ABSTRACT

The present disclosure provides an isolated monoclonal antibody that specifically binds human SIRPα, or an antigen-binding portion thereof. A nucleic acid molecule encoding the antibody or the antigen-binding portion thereof, an expression vector, a host cell and a method for expressing the antibody or the antigen-binding portion thereof are also provided. The present disclosure further provides a bispecific molecule, an immunoconjugate, a chimeric antigen receptor, an oncolytic virus and a pharmaceutical composition comprising the antibody or the antigen-binding portion thereof, as well as a treatment method using an anti-SIRPα antibody or the antigen-binding portion thereof.

This application claims priority to International Patent Application No. PCT/CN2022/088000 filed on Apr. 20, 2022.

### FIELD OF THE INVENTION

The present disclosure relates generally to an isolated monoclonal antibody, particularly a chimeric or fully human monoclonal antibody, or an antigen-binding portion thereof, that binds to SIRPα, with high affinity and functionality. A nucleic acid molecule encoding the antibody or the antigen-binding portion thereof, an expression vector, a host cell and a method for expressing the antibody or the antigen-binding portion thereof are also provided. The present disclosure further provides a bispecific molecule, an immunoconjugate, a chimeric antigen receptor, an oncolytic virus, and a pharmaceutical composition which may comprise the antibody or the antigen-binding portion thereof, as well as a treatment method using the anti-SIRPα antibody or the antigen-binding portion thereof of the disclosure.

### BACKGROUND OF THE INVENTION

Immune checkpoints are regulators of the immune system, where immune cells must pass stimulatory and/or inhibitory immune checkpoints, to proceed with activation or anergy.

Inhibitory immune checkpoints are especially important for self-tolerance, preventing the immune system from attacking normal cells. They are often manipulated by e.g., tumor cells to evade immune surveillance. Over the past decades, inhibitory immune checkpoint blockade has been extensively studied in e.g., cancer immunotherapy, and inhibitors of PD-1 and/or CTLA-4 have been clinically approved for treatment of various cancers.

Signal-regulatory protein alpha (SIRP-alpha, also called SIRPα) is an inhibitory immune checkpoint for innate immune cells, predominantly expressed on myeloid cells, including macrophages, dendritic cells and neutrophils. It contains in its cytoplasmic domain immunoreceptor tyrosine-based inhibition motifs (ITIMs). Upon binding of CD47, a membrane protein expressed on almost all cell types, to SIRPα, the ITIMs become phosphorylated and recruit inhibitory molecules such as SHP-1 and SHP-2, exerting inhibitory effects on cell activation (Veillette A, Chen J. (2018) Trends Immunol. 39(3):173-184). A lot of tumor cells overexpress CD47 molecules to suppress phagocytosis by immune cells.

Ten human SIRPα alleles have been found till now, human SIRPα isoform 1 (or V1), isoform 2 (or V2) and isoform 8 (or V8) account for 80% of those expressed in human.

SIRPα has two close SIRP family members, SIRPβ expressed on macrophages and neutrophils, and SIRPγ expressed on lymphocytes and natural killer cells. SIRPβ does not bind to CD47, but is a stimulatory receptor, while SIRPγ binds CD47 with a low affinity but is a non-signaling receptor (Barclay, A. N. and Brown, M. H. (2006) Nat. Rev. Immunol. 6(6): 457-464; Takahashi S. (2018) Biomedical Reports. 9(1):3-7).

Preclinical studies have shown the disruption of SIRPα-CD47 interaction can promote phagocytic uptake of cancer cells by macrophages, e.g., with the aids of pro-phagocytic receptors on cancer cells such as SLAMF7 and Mac-1 (Chen, J. et al. (2017) 544:493-497). The antigenic peptides from the cancer cells as generated during phagocytosis may later initiate an adaptive immune response. Most pharmaceuticals for SIRPα-CD47 blockade currently under clinical trials target CD47 molecules and are capable of inducing FcR-mediated phagocytosis, such as recombinant SIRPα-Fc fusion proteins, or IgG1 anti-CD47 antibodies. Such agents may also harm normal CD47-expressing hematopoietic and non-hematopoietic cells, causing anemia, thrombocytopenia, and/or leukopenia, as observed in nonhuman models (Veillette A, Chen J. (2018) supra). Therefore, there may be some benefits in using blocking antibodies against SIRPα which has a more restricted histological distribution, resulting in limited toxicity. It was even found SIRPα is aberrantly expressed on some solid tumors, including renal cell carcinoma and melanoma, anti-SIRPα antibodies may directly initiate FcR-mediated phagocytosis of these tumor cells (Yanagita, T. et al. (2017) JCI Insight 2(1): e89140).

KWAR23, an anti-SIRPα antibody that binds SIRPα at an epitope on CD47-SIRPα interface, was reported to enhance macrophage-mediated phagocytosis of colorectal adenocarcinoma cells in vitro when used alone or in combination with Cetuximab, in a concentration dependent manner. BI 765063 (BOehringer Ingelheim/OSE), an anti-SIRPα antibody that selectively inhibits SIRPα and restores the activity of tumor associated macrophages and dendritic cells in response to tumor cells, is now being tested in patients with advanced solid tumors in the Phase 1 clinical trial as a single agent and in combination with an PD-1 antagonist. CC-95251 (Celgene) is also under Phase 1 trial for treatment of advanced solid and hematologic cancers alone or in combination with Cetuximab or Rituximab (Uger R, Johnson L. (2020) Expert Opin Biol Ther. 20(1):5-8; Zhang W et al. (2020) Front Immunol. 11:18).

Studies have indicated that the blocking antibodies against SIRPα may affect the function of other members of the SIRP family, and the polymorphisms in human SIRPα may make the antibodies less efficacious in some subjects (Yanagita, T. et al. (2017) supra; Barclay, A. N. and Van den Berg, T. K. (2014) Annu. Rev. Immunol. 32: 25-50). Therefore, there is always a need for more monoclonal antibodies with desired characteristics.

Citation or identification of any document in this application is not an admission that such document is available as prior art to the present invention.

### SUMMARY OF THE INVENTION

The inventors of the present application have found an antagonistic anti-SIRPα antibody or an antigen-binding portion thereof that (a) specifically binds to SIRPα (e.g., human and monkey SIRPα, including isoform 1 (V1), isoform 2 (V2) and isoform 8 (V8)) with comparable, if not higher, binding affinity/activity as compared to prior art anti-SIRPα antibodies such as KWAR23 and BI-765063; (b) cross reacts with SIRPβ, with comparable, if not higher, binding affinity/activity compared to prior art anti-SIRPα antibodies such as KWAR23 and BI-765063; (c) does not bind SIRPγ; (d) does not bind mouse SIRPα, (e) blocks SIRPα binding to CD47 with comparable, if not higher, blocking activity compared to the prior art antibodies, (f) can be internalized by SIRPα-expressing cells, (g) induces phagocytosis of tumor cells by macrophages with comparable, if not higher, activity compared to prior art anti-SIRPα antibodies such as KWAR23 and BI-765063, and/or (h) shows in vivo anti-tumor activity.

The antibody or antigen-binding portion of the disclosure can be used for a variety of applications, including detection of human or monkey SIRPα proteins in vitro, and treatment of cancers, including solid and hematological cancers.

Accordingly, in one aspect, the disclosure pertains to an isolated monoclonal antibody (e.g., a chimeric or fully human antibody), or an antigen-binding portion thereof, that binds SIRPα, comprising (i) a heavy chain variable region that may comprise a VH CDR1 region, a VH CDR2 region and a VH CDR3 region, wherein the VH CDR1 region, the VH CDR2 region and the VH CDR3 region may comprise amino acid sequences having at least 85%, 86%, 87%, 88%, 89%, 90%, 91%, 92%, 93%, 94%, 95%, 96%, 97%, 98%, 99% or 100% identity to (1) SEQ ID NOs: 1, 2 (X1=N, X2=G; X1=N, X2=A; X1=A, X2=G) and 3, respectively; (2) SEQ ID NOs: 9 (X=Y), 10 (X1=N, X2=G; X1=N, X2=A; X1=A, X2=G), and 11, respectively; or (3) SEQ ID NOs: 9 (X=F), 10 (X1=N, X2=G) and 11, respectively; and/or (ii) a light chain variable region that may comprise a VL CDR1 region, a VL CDR2 region and a VL CDR3 region, wherein the VL CDR1 region, the VL CDR2 region and the VL CDR3 region may comprise amino acid sequences having at least 85%, 86%, 87%, 88%, 89%, 90%, 91%, 92%, 93%, 94%, 95%, 96%, 97%, 98%, 99% or 100% identity to (1) SEQ ID NOs: 4, 5 (X=N; X=A) and 6, respectively; or (2) SEQ ID NOs: 12, 13 and 14, respectively.

The isolated monoclonal antibody, or the antigen-binding portion thereof, of the present disclosure may comprise a heavy chain variable region comprising a VH CDR1 region, a VH CDR2 region and a VH CDR3 region, and a light chain variable region comprising a VL CDR1 region, a VL CDR2 region and a VL CDR3 region, wherein the VH CDR1, VH CDR2, VH CDR3, VL CDR1, VL CDR2 and VL CDR3 may comprise amino acid sequences having at least 85%, 86%, 87%, 88%, 89%, 90%, 91%, 92%, 93%, 94%, 95%, 96%, 97%, 98%, 99% or 100% identity to (1) SEQ ID NOs: 1, 2 (X1=N, X2=G), 3, 4, 5 (X=N) and 6, respectively; (2) SEQ ID NOs: 1, 2 (X1=N, X2=A; or X1=A, X2=G), 3, 4, 5 (X=A) and 6, respectively; (3) SEQ ID NOs: 9 (X=Y), 10 (X1=N, X2=G; X1=N, X2=A; X1=A, X2=G), 11, 12, 13 and 14, respectively; or (4) SEQ ID NOs: 9 (X=F), 10 (X1=N, X2=G), 11, 12, 13 and 14, respectively.

The isolated monoclonal antibody, or the antigen-binding portion thereof, of the present disclosure may comprise a heavy chain variable region that may comprise an amino acid sequence having at least 85%, 86%, 87%, 88%, 89%, 90%, 91%, 92%, 93%, 94%, 95%, 96%, 97%, 98%, 99% or 100% identity to SEQ ID NOs: 7 (X1=N, X2=G; X1=N, X2=A; X1=A, X2=G), 15 (X1=N, X2=G; X1=N, X2=A; X1=A, X2=G), or 17.

The isolated monoclonal antibody, or the antigen-binding portion thereof, of the present disclosure may comprise a light chain variable region that may comprise an amino acid sequence having at least 85%, 86%, 87%, 88%, 89%, 90%, 91%, 92%, 93%, 94%, 95%, 96%, 97%, 98%, 99% or 100% identity to SEQ ID NOs: 8 (X=N; X=A), 16 (X=N; X=S), or 18.

The isolated monoclonal antibody, or the antigen-binding portion thereof, of the present disclosure may comprise a heavy chain variable region and a light chain variable region, the heavy chain variable region and the light chain variable region may comprise amino acid sequences having at least 85%, 86%, 87%, 88%, 89%, 90%, 91%, 92%, 93%, 94%, 95%, 96%, 97%, 98%, 99% or 100% identity to (1) SEQ ID NOs: 7 (X1=N, X2=G) and 8 (X=N), respectively; (2) SEQ ID NOs: 7 (X1=N, X2=A) and 8 (X=A), respectively; (3) SEQ ID NOs: 7 (X1=A, X2=G) and 8 (X=A), respectively; (4) SEQ ID NOs: 15 (X1=N, X2=G) and 16 (X=N), respectively; (5) SEQ ID NOs: 15 (X1=N, X2=A) and 16 (X=S), respectively; (6) SEQ ID NOs: 15 (X1=A, X2=G) and 16 (X=S), respectively; or (7) SEQ ID NOs: 17 and 18, respectively.

The isolated monoclonal antibody, or the antigen-binding portion thereof, of the present disclosure may comprise a heavy chain and a light chain linked by disulfide bonds, the heavy chain may comprise a heavy chain variable region and a heavy chain constant region, the light chain may comprise a light chain variable region and a light chain constant region, wherein the C terminus of the heavy chain variable region is linked to the N terminus of the heavy chain constant region, and the C terminus of the light chain variable region is linked to the N terminus of the light chain constant region, wherein the heavy chain variable region and the light chain variable region may comprise amino acid sequences described above, and the antibody or antigen-binding portion thereof binds to SIRPα.

The heavy chain constant region may be one with FcR binding affinity, such as genetically engineered human IgG4 constant region, or human IgG1 constant region. The heavy chain constant region may be modified to have enhanced FcR binding affinity and/or enhanced antibody structure stability. In certain embodiments, the heavy chain constant region may be human IgG4 constant region having the amino acid sequence set forth in e.g., SEQ ID NO: 19, or a functional fragment thereof. In certain embodiments, the heavy chain constant region may be human IgG1 constant region having the amino acid sequence set forth in e.g., SEQ ID NO: 38 (X1=M, X2=S, X3=T; X1=Y, X2=T, X3=E). The heavy chain constant region may also be one with weak or no FcR binding affinity, such as IgG4 constant region, or modified IgG1 constant region. The light chain constant region may be human kappa constant region having the amino acid sequences set forth in e.g., SEQ ID NO.: 20.

The antibody of the present disclosure in certain embodiments may comprise or consist of two heavy chains and two light chains, wherein each heavy chain may comprise the heavy chain constant region, heavy chain variable region or CDR sequences mentioned above, and each light chain may comprise the light chain constant region, light chain variable region or CDR sequences mentioned above, wherein the antibody binds to SIRPα. The antibody or the antigen-binding portion thereof of the present disclosure in other embodiments may be a single chain variable fragment (scFv) antibody, or antibody fragments, such as Fab or F(ab′)2 fragments.

The disclosure also provides a bispecific molecule that may comprise the antibody, or the antigen-binding portion thereof, of the disclosure, linked to a second functional moiety (e.g., a second antibody) having a different binding specificity than said antibody, or antigen-binding portion thereof, e.g., a second functional moiety targeting a tumor associated antigen, such as CD19, CD20, CD22, CD4, CD24, CD38, CD123, CD228, CD138, BCMA, GPC3, CEA, folate receptor (FRα), mesothelin, CD276, gp100, 5T4, GD2, EGFR, MUC-1, PSMA, EpCAM, MCSP, SM5-1, MICA, MICB, ULBP, and HER-2. The disclosure also provides an immunoconjugate, such as an antibody-drug conjugate, that may comprise an antibody, or antigen-binding portion thereof, of the disclosure, linked to a therapeutic agent, such as a cytotoxin, e.g., a recombinant protein termed DT3C having the amino acid sequence of SEQ ID NO: 36. In another aspect, the antibody or the antigen binding portion thereof of the present disclosure can be made into part of a chimeric antigen receptor (CAR). Also provided is an immune cell that may comprise the antigen chimeric receptor, such as a T cell and a NK cell. The antibody or the antigen binding portion thereof of the present disclosure can also be encoded by or used in conjunction with an oncolytic virus.

A nucleic acid molecule encoding the antibody, or the antigen-binding portion thereof, of the disclosure is also encompassed by the disclosure, as well as an expression vector that may comprise such a nucleic acid molecule and a host cell that may comprise such an expression vector or have the nucleic acid molecule integrated into its genome. A method for preparing the anti-SIRPα antibody or the antigen-binding portion thereof of the disclosure using the host cell is also provided, that may comprise steps of (i) expressing the antibody in the host cell and (ii) isolating the antibody from the host cell or its cell culture.

A composition, e.g., a pharmaceutical composition, that may comprise the antibody, or the antigen-binding portion thereof, or the immunoconjugate, the bispecific molecule, the immune cell with CAR, the oncolytic virus, the nucleic acid molecule, the expression vector or the host cell of the disclosure, and optionally a pharmaceutically acceptable carrier, is also provided. In certain embodiments, the pharmaceutical composition may further contain a therapeutic agent for treating cancers, such as an anti-cancer agent. In certain embodiments, the composition further comprises an anti-Claudin18.2 antibody, or an anti-CD20 antibody.

In yet another aspect, the disclosure provides a method for treating a cancer in a subject in need thereof, which may comprise administering to the subject a therapeutically effective amount of the composition of the present disclosure. The cancer may be a solid cancer or a hematologic cancer, including but not limited to, non-small lung cancer, breast cancer, ovarian cancer, renal cell cancer, colorectal cancer and pancreatic cancer. The composition may comprise the antibody, or the antigen-binding portion thereof, the bispecific molecule, the immunoconjugate, the immune cell with CAR, the oncolytic virus, the nucleic acid molecule, the expression vector or the host cell of the disclosure. If the composition comprises the antibody or the antigen binding portion thereof with strong FcR binding affinity, the immunoconjugate, the immune cell with CAR, or the oncolytic virus, then local delivery of the composition to the tumor site(s) is preferred. In some embodiments, at least one additional anti-cancer antibody can be further administered, such as an antibody targeting a tumor associated antigen such as CD19, CD20, CD22, CD4, CD24, CD38, CD123, CD228, CD138, BCMA, GPC3, CEA, folate receptor (FRα), mesothelin, CD276, gp100, 5T4, GD2, EGFR, MUC-1, PSMA, EpCAM, MCSP, SM5-1, MICA, MICB, ULBP, and HER-2, or an antibody targeting an inhibitory immune checkpoint, such as LAG-3, PD-1, VISTA, or CTLA-4. In yet another embodiment, an antibody, or an antigen-binding portion thereof, of the disclosure is administered with a cytokine (e.g., IL-2 and/or IL-21), or a costimulatory antibody (e.g., an anti-CD137 and/or anti-GITR antibody). In another embodiment, an antibody, or an antigen-binding portion thereof, of the disclosure is administered with a chemotherapeutic agent, which may be a cytotoxic agent, such as epirubicin, oxaliplatin, and/or 5-fluorouracil (5-FU). In certain embodiments, the antibody or antigen binding portion thereof of the disclosure may be administered with an anti-Claudin18.2 antibody or an anti-CD20 antibody. The antibody or antigen binding portion thereof of the present disclosure may be, for example, chimeric or fully human. In certain embodiments, the subject is human.

In yet another aspect, the disclosure provides a method of modulating or enhancing an immune response in a subject in need thereof, comprising administering to the subject the composition of the disclosure such that the immune response in the subject is modulated/enhanced. The composition comprises the antibody, or the antigen-binding portion thereof, with weak FcR binding heavy chain constant regions, the bispecific molecule, the nucleic acid molecule, or the expression vector of the disclosure. The antibody or antigen binding portion thereof of the present disclosure may be, for example, chimeric or fully human.

In certain embodiments, the subject is human.

Other features and advantages of the instant disclosure will be apparent from the following detailed description and examples, which should not be construed as limiting. The contents of all references, Genbank entries, patents and published patent applications cited throughout this application are expressly incorporated herein by reference.

Accordingly, it is an object of the invention not to encompass within the invention any previously known product, process of making the product, or method of using the product such that Applicants reserve the right and hereby disclose a disclaimer of any previously known product, process, or method. It is further noted that the invention does not intend to encompass within the scope of the invention any product, process, or making of the product or method of using the product, which does not meet the written description and enablement requirements of the USPTO (35 U.S.C. § 112, first paragraph) or the EPO (Article 83 of the EPC), such that Applicants reserve the right and hereby disclose a disclaimer of any previously described product, process of making the product, or method of using the product. It may be advantageous in the practice of the invention to be in compliance with Art. 53(c) EPC and Rule 28(b) and (c) EPC. All rights to explicitly disclaim any embodiments that are the subject of any granted patent(s) of applicant in the lineage of this application or in any other lineage or in any prior filed application of any third party is explicitly reserved. Nothing herein is to be construed as a promise.

It is noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

### BRIEF DESCRIPTION OF THE DRAWINGS

The following detailed description, given by way of example, but not intended to limit the invention solely to the specific embodiments described, may best be understood in conjunction with the accompanying drawings.

FIG. 1 shows the binding capability of antibodies E1D5E1F1, E1F1B1C3 and E1H10B7C7 to human SIRPα in a capture ELISA.

FIG. 2 shows the binding capability of antibodies E1D5E1F1, E1F1B1C3 and E1H10B7C7 to monkey SIRPα in an indirect ELISA.

FIG. 3 shows the binding capability of antibodies E1D5E1F1, E1F1B1C3 and E1H10B7C7 to human SIRPα-isoform2 in an indirect ELISA.

FIG. 4 shows the binding capability of antibodies E1D5E1F1, E1F1B1C3 and E1H10B7C7 to HEK293 cells expressing human SIRPα in a cell based binding FACS assay.

FIG. 5 shows the binding capability of antibodies E1D5E1F1, E1F1B1C3 and E1H10B7C7 to HEK293 cells expressing human SIRPβ in a cell based binding FACS assay.

FIG. 6 shows the binding capability of antibodies E1D5E1F1, E1F1B1C3 and E1H10B7C7 to Jurkat cells expressing human SIRPγ in a cell based binding FACS assay.

FIG. 7 shows the ability of antibodies E1D5E1F1, E1F1B1C3 and E1H10B7C7 to block binding of human CD47 to cell surface SIRPα in a cell based blocking FACS assay.

FIG. 8 shows the ability of antibodies E1D5E1F1, E1F1B1C3 and E1H10B7C7 to block human SIRPα-CD47 binding in a competitive ELISA.

FIG. 9 shows the binding capability of E1H10B7-CDRV1-IgG1 (YTE) and E1H10B7-CDRV1-IgG1 to monkey SIRPα in an indirect ELISA.

FIG. 10 shows the binding capability of antibodies E1F1B1C3-CDRV1-IgG1 (YTE) and E1F1B1C3-CDRV1-IgG1 to monkey SIRPα in an indirect ELISA.

FIG. 11 shows the binding capability of antibodies E1H10B7-CDRV1-IgG1 (YTE) and E1H10B7-CDRV1-IgG1 to mouse SIRPα in an indirect ELISA.

FIG. 12 shows the binding capability of antibodies E1F1B1C3-CDRV1-IgG1 (YTE) and E1F1B1C3-CDRV1-IgG1 to mouse SIRPα in an indirect ELISA.

FIG. 13 shows the binding capability of antibodies E1H10B7-CDRV1-IgG1 (YTE) and E1H10B7-CDRV1-IgG1 to human SIRPα-V8 in an indirect ELISA.

FIG. 14 shows the binding capability of antibodies E1F1B1C3-CDRV1-IgG1 (YTE) and E1F1B1C3-CDRV1-IgG1 to human SIRPα-V8 in an indirect ELISA.

FIG. 15 shows the binding capability of antibodies E1H10B7-CDRV1-IgG1 (YTE) and E1H10B7-CDRV1-IgG1 to human SIRPα-isoform2 in an indirect ELISA.

FIG. 16 shows the binding capability of antibodies E1F1B1C3-CDRV1-IgG1 (YTE) and E1F1B1C3-CDRV1-IgG1 to human SIRPα-isoform2 in an indirect ELISA.

FIG. 17 shows the binding capability of antibodies E1H10B7-CDRV1-IgG1 (YTE) and E1H10B7-CDRV1-IgG to 293F cells expressing human SIRPα in a cell based binding FACS assay.

FIG. 18 shows the binding capability of antibodies E1F1B1C3-CDRV1-IgG1 (YTE) and E1F1B1C3-CDRV1-IgG1 to 293F cells expressing human SIRPα in a cell based binding FACS assay.

FIG. 19 shows the binding capability of antibodies E1H10B7-CDRV1-IgG1 (YTE) and E1H10B7-CDRV1-IgG to 293F cells expressing human SIRPβ in a cell based binding FACS assay.

FIG. 20 shows the binding capability of antibodies E1F1B1C3-CDRV1-IgG1 (YTE) and E1F1B1C3-CDRV1-IgG1 to 293F cells expressing human SIRPβ in a cell based binding FACS assay.

FIG. 21 shows the binding capability of antibodies E1H10B7-CDRV1-IgG1 (YTE) and E1H10B7-CDRV1-IgG to Jurkat cells expressing human SIRPγ in a cell based binding FACS assay.

FIG. 22 shows the binding capability of antibodies E1F1B1C3-CDRV1-IgG1 (YTE) and E1F1B1C3-CDRV1-IgG1 to Jurkat cells expressing human SIRPγ in a cell based binding FACS assay.

FIG. 23 shows the ability of antibodies E1H10B7-CDRV1-IgG1 (YTE) and E1H10B7-CDRV1-IgG to block human CD47 binding to cell surface SIRPα in a cell based blocking FACS assay.

FIG. 24 shows the ability of antibodies E1F1B1C3-CDRV1-IgG1 (YTE) and E1F1B1C3-CDRV1-IgG1 to block human CD47 binding to cell surface SIRPα in a cell based blocking FACS assay.

FIG. 25 shows the ability of antibodies E1H10B7-CDRV1-IgG1 (YTE) and E1H10B7-CDRV1-IgG to block human SIRPα binding to cell surface CD47 in a cell based blocking FACS assay.

FIG. 26 shows the ability of antibodies E1F1B1C3-CDRV1-IgG1 (YTE) and E1F1B1C3-CDRV1-IgG1 to block human SIRPα binding to cell surface CD47 in a cell based blocking FACS assay.

FIG. 27 shows the ability of antibodies E1H10B7-CDRV1-IgG1 (YTE) and E1F1B1C3-CDRV1-IgG1 (YTE) to induce macrophage-mediated phagocytosis of Raji cells.

FIG. 28 shows the effect of antibodies E1H10B7-CDRV1-IgG1 and E1F1B1C3-CDRV1-IgG1 on SIRPα phosphorylation in THP1 cells.

FIG. 29 shows the effect of antibodies E1H10B7-CDRV1-IgG1, E1H10B7-CDRV1-IgG1 (YTE), E1F1B1C3-CDRV1-IgG1 and E1 F1B1C3-CDRV1-IgG1 (YTE) on CD47-mediated SIRPα phosphorylation in THP1 cells.

FIG. 30 shows the internalization of antibodies E1H10B7-CDRV1-IgG1, E1H10B7-CDRV1-IgG1 (YTE), E1F1B1C3-CDRV1-IgG1 and E1F1B1C3-CDRV1-IgG1 (YTE) by SIRPα-expressing cells.

FIG. 31 shows the body weights of B-NDG hSIRPA mice injected with B-luc-GFP-Raji lymphoma cells and treated by the antibodies of the disclosure.

FIG. 32 shows the tumor signal intensity in B-NDG hSIRPA mice injected with B-luc-GFP-Raji lymphoma cells and treated by the antibodies of the disclosure.

FIG. 33 shows the tumor signal intensity in B-NDG hSIRP mice injected with B-luc-GFP-Raji lymphoma cells and treated by the antibodies of the disclosure at different doses.

FIG. 34 shows the tumor sizes in C57BL/6-hSIRPA(2)/hCD47 mice injected with murine colon cancer cells and treated by the antibodies of the disclosure.

### DETAILED DESCRIPTION OF THE INVENTION

To ensure that the present disclosure may be more readily understood, certain terms are first defined. Additional definitions are set forth throughout the detailed description.

The term “SIRPα” refers to signal regulatory protein alpha, also known as CD172a or Src homology 2 domain-containing phosphatase substrate-1. The term “SIRPα” may comprise variants, isoforms, homologs, orthologs and paralogs. For example, an antibody specific for a human SIRPα protein may, in certain cases, cross-react with a SIRPα protein from a species other than human, such as monkey. In other embodiments, an antibody specific for a human SIRPα protein may be completely specific for the human SIRPα protein and exhibit no cross-reactivity to other species or of other types, or may cross-react with SIRPα from certain other species but not all other species.

The term “human SIRPα” refers to a SIRPα protein having an amino acid sequence from a human, such as the amino acid sequence of human SIRPα with NCBI Reference No.: NP_001035111.1 (Burgess T L et al., (2020), PLoS ONE15(4): e0226661). The terms “monkey SIRPα” or “cynomolgus SIRPα” refer to a SIRPα protein having an amino acid sequence from monkeys, such as the amino acid sequence with NCBI Reference No.: NP_001271679.1 (Wang H Y et al., (2007), PLoS Biol. 5(2): e13). The term “mouse SIRPα” refers to a SIRPα protein having an amino acid sequence from a mouse, such as the amino acid sequence of mouse SIRPα with GenBank Reference No.: AAH62197.1 (Strausberg, R. L. et al., (2002) Proc. Natl. Acad. Sci. U.S.A. 99(26): 16899-16903).

The term “antibody” as used herein refers to an immunoglobulin molecule that recognizes and specifically binds a target, such as SIRPα, through at least one antigen-binding site wherein the antigen-binding site is usually within the variable region of the immunoglobulin molecule. As used herein, the term encompasses intact polyclonal antibodies, intact monoclonal antibodies, single-chain Fv (scFv) antibodies, heavy chain antibodies (HCAbs), light chain antibodies (LCAbs), multi-specific antibodies, bispecific antibodies, monospecific antibodies, monovalent antibodies, fusion proteins comprising an antigen-binding site of an antibody, and any other modified immunoglobulin molecule comprising an antigen-binding site (e.g., dual variable domain immunoglobulin molecules) as long as the antibodies exhibit the desired biological activity. Antibodies also include, but are not limited to, mouse antibodies, chimeric antibodies, humanized antibodies, and human antibodies. An antibody can be any of the five major classes of immunoglobulins: IgA, IgD, IgE, IgG, and IgM, or subclasses (isotypes) thereof (e.g., IgG1, IgG2, IgG3, IgG4, IgA1 and IgA2), based on the identity of their heavy-chain constant domains referred to as alpha, delta, epsilon, gamma, and mu, respectively. The different classes of immunoglobulins have different and well-known subunit structures and three-dimensional configurations. Antibodies can be naked or conjugated to other molecules, including but not limited to, toxins and radioisotopes. Unless expressly indicated otherwise, the term “antibody” as used herein include “antigen-binding portion” of the intact antibodies. An IgG is a glycoprotein which may comprise two heavy (H) chains and two light (L) chains inter-connected by disulfide bonds. Each heavy chain may be comprised of a heavy chain variable region (abbreviated herein as VH) and a heavy chain constant region. The heavy chain constant region may be comprised of three domains, CH1, CH2 and CH3. Each light chain may be comprised of a light chain variable region (abbreviated herein as VL) and a light chain constant region. The light chain constant region may be comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxyl-terminus in the following order: FR1, CDR1, FR2, CDR2, FR3, CDR3, FR4. The variable regions of the heavy and light chains contain a binding domain that interacts with an antigen. The constant regions of the antibodies can mediate the binding of the immunoglobulin to host tissues or factors, including various cells of the immune system (e.g., effector cells) and the first component (C1q) of the classical complement system.

The term “antigen-binding portion” of an antibody (or simply “antibody portion”), as used herein, refers to one or more fragments of an antibody that retain the ability to specifically bind to an antigen (e.g., a SIRPα protein). It has been shown that the antigen-binding function of an antibody can be performed by fragments of a full-length antibody. Examples of binding fragments encompassed within the term “antigen-binding portion” of an antibody include (i) a Fab fragment, a monovalent fragment consisting of the VL, VH, CL and CH1 domains; (ii) a F(ab′)2 fragment, a bivalent fragment which may comprise two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; (vi) an isolated complementarity determining region (CDR); and (viii) a nanobody, a heavy chain variable region containing a single variable domain and two constant domains. Furthermore, although the two domains of the Fv fragment, VL and VH, are coded by separate genes, they can be joined, using recombinant methods, by a synthetic linker that enables them to be made as a single protein chain in which the VL and VH regions pair to form monovalent molecules. Such single chain antibodies are also intended to be encompassed within the term “antigen-binding portion” of an antibody. These antibody fragments are obtained using conventional techniques known to those with skill in the art, and the fragments are screened for utility in the same manner as are intact antibodies.

An “isolated antibody”, as used herein, is intended to refer to an antibody that is substantially free of other antibodies having different antigenic specificities (e.g., an isolated antibody that specifically binds a SIRPα protein is substantially free of antibodies that specifically bind antigens other than SIRPα proteins). An isolated antibody that specifically binds a human SIRPα protein may, however, have cross-reactivity to other antigens, such as SIRPα proteins from other species. Moreover, an isolated antibody can be substantially free of other cellular material and/or chemicals.

The term “monoclonal antibody” as used herein refers to an antibody obtained from a population of substantially homogeneous antibodies, i.e., the individual antibodies comprising the population are identical except for possible naturally occurring mutations and/or post-translation modifications (e.g., isomerizations, amidations) that may be present in minor amounts. Monoclonal antibodies are highly specific, being directed against a single antigenic site. In contrast to polyclonal antibody preparations which typically include different antibodies directed against different determinants (epitopes), each monoclonal antibody is directed against a single determinant on the antigen. In addition to their specificity, the monoclonal antibodies are advantageous in that they are synthesized by the hybridoma culture, uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including, for example, the hybridoma method.

The term “human antibody” or “fully human antibody”, as used herein, is intended to include antibodies having variable regions in which both the framework and CDR regions are derived from human germline immunoglobulin sequences. Furthermore, if the antibody contains a constant region, the constant region also is derived from human germline immunoglobulin sequences. The human antibodies of the disclosure can include amino acid residues not encoded by human germline immunoglobulin sequences (e.g., mutations introduced by random or site-specific mutagenesis in vitro or by somatic mutation in vivo). However, the term “human antibody”, as used herein, is not intended to include antibodies in which CDR sequences derived from the germline of another mammalian species have been grafted onto human framework sequences.

The term “chimeric antibody” refers to an antibody made by combining genetic material from a nonhuman source with genetic material from a human being. Or more generally, a chimeric antibody is an antibody having genetic material from a certain species with genetic material from another species.

The term “isotype” refers to the antibody class (e.g., IgM or IgG1) that is encoded by the heavy chain constant region genes.

The phrases “an antibody recognizing an antigen” and “an antibody specific for an antigen” are used interchangeably herein with the term “an antibody which binds specifically to an antigen.”

As used herein, an antibody that “specifically binds to human SIRPα” is intended to refer to an antibody that binds to human SIRPα protein (and possibly a SIRPα protein from one or more non-human species) but does not substantially bind to non-SIRPα proteins. Preferably, the antibody binds to human SIRPα protein with “high affinity”, namely with a K_{D} of 1.0×10⁻⁷ M or less, more preferably 5.0×10⁻⁸ M or less, and more preferably 1.0×10⁻⁸ M or less.

The term “does not substantially bind” to a protein or cells, as used herein, means does not bind or does not bind with a high affinity to the protein or cells, i.e. binds to the protein or cells with a K_{D} of 1.0×10⁻⁶ M or more, more preferably 1.0×10⁻⁵ M or more, more preferably 1.0×10⁻⁴ M or more, more preferably 1.0×10⁻³ M or more, even more preferably 1.0×10⁻² M or more.

The term “high affinity” for an IgG antibody refers to an antibody having a K_{D} of 1.0×10⁻⁷ M or less, more preferably 5.0×10⁻⁸ M or less for a target antigen. However, “high affinity” binding can vary for other antibody isotypes. For example, “high affinity” binding for an IgM isotype refers to an antibody having a K_{D} of 10⁻⁶ M or less, more preferably 10⁻⁷ M or less, even more preferably 10⁻⁸ M or less.

The term “K_{assoc}” or “Kₐ”, as used herein, is intended to refer to the association rate of a particular antibody-antigen interaction, whereas the term “K_{dis}” or “K_{d}”, as used herein, is intended to refer to the dissociation rate of a particular antibody-antigen interaction. The term “K_{D}”, as used herein, is intended to refer to the dissociation constant, which is obtained from the ratio of K_{d} to Kₐ (i.e., K_{d}/Kₐ) and is expressed as a molar concentration (M). K_{D} values for antibodies can be determined using methods well established in the art. A preferred method for determining the K_{D} of an antibody is by using surface plasmon resonance, preferably using a biosensor system such as a Biacore™ system.

The term “EC₅₀”, also known as half maximal effective concentration, refers to the concentration of an antibody which induces a response halfway between the baseline and maximum after a specified exposure time.

The term “IC₅₀”, also known as half maximal inhibitory concentration, refers to the concentration of an antibody which inhibits a specific biological or biochemical function by 50% relative to the absence of the antibody.

The term “antagonistic” means the antibody or antigen binding portion thereof of the disclosure can bind to SIRPα and block SIRPα signaling initiated by CD47 engagement.

The term “subject” includes any human or nonhuman animal. The term “nonhuman animal” includes all vertebrates, e.g., mammals and non-mammals, such as non-human primates, sheep, dogs, cats, cows, horses, chickens, amphibians, and reptiles, although mammals are preferred, such as non-human primates, sheep, dogs, cats, cows and horses.

The term “therapeutically effective amount” means an amount of the antibody or the antigen binding portion of the present disclosure sufficient to prevent or ameliorate the symptoms associated with a disease or condition (such as a chronic inflammation) and/or lessen the severity of the disease or condition. A therapeutically effective amount is understood to be in context to the condition being treated, where the actual effective amount is readily discerned by those of skill in the art.

The antibody, or the antigen-binding portion thereof, of the disclosure (a) specifically binds SIRPα (e.g., human and monkey SIRPα) with comparable, if not higher, binding affinity/activity as compared to prior art anti-SIRPα antibodies such as KWAR23 and BI-765063; (b) cross reacts with SIRPβ, with comparable, if not higher, binding affinity/activity compared to prior art anti-SIRPα antibodies such as KWAR23 and BI-765063; (c) does not bind SIRPγ; (d) does not bind mouse SIRPα, (e) blocks SIRPα binding to CD47 with comparable, if not higher, blocking activity compared to the prior art antibodies, (f) can be internalized by SIRPα-expressing cells, and/or (g) shows in vivo anti-tumor activity. The antibodies of the disclosure are chimeric and human monoclonal antibodies.

The antibody or antigen binding portion thereof of the disclosure is structurally and chemically characterized below and in the following Examples. The amino acid sequence ID NOs of the heavy/light chain variable regions and CDRs of the disclosure are summarized in Table 1 below, some antibodies sharing the same VH or VL. The heavy chain constant region for the antibodies may be human IgG4 heavy chain constant region having an amino acid sequence set forth in, e.g., SEQ ID NO: 19, human IgG1 heavy chain constant region having the amino acid sequence of SEQ ID NO: 38 (X1=M, X2=S, X3=T; X1=Y, X2=T, X3=E), modified to have enhance FcR binding affinity and/or antibody stability. The heavy chain constant region may also with weak FcR binding affinity. The light chain constant region for the antibodies may be human kappa constant region having an amino acid sequence set forth in, e.g., SEQ ID NO: 20. The antibodies of the disclosure may also contain human lambda light chain constant region.

The heavy chain variable region CDRs and the light chain variable region CDRs in Table 1 have been defined by the Kabat numbering system. However, as is well known in the art, CDR regions can also be determined by other systems such as Chothia, and IMGT, AbM, or Contact numbering system/method, based on heavy chain/light chain variable region sequences.

The VH and VL sequences (or CDR sequences) of other Anti-SIRPα antibodies which bind to human SIRPα can be “mixed and matched” with the VH and VL sequences (or CDR sequences) of the anti-SIRPα antibody of the present disclosure. Preferably, when VH and VL chains (or the CDRs within such chains) are mixed and matched, a VH sequence from a particular VH/VL pairing is replaced with a structurally similar VH sequence. Likewise, preferably a VL sequence from a particular VH/VL pairing is replaced with a structurally similar VL sequence.

TABLE 1
Amino acid sequence ID NOs of heavy/light chain variable regions and CDRs

| | VH CDR1 | VH CDR2 | VH CDR3 | VH | VL CDR1 | VL CDR2 | VL CDR3 | VL |
|---|---|---|---|---|---|---|---|---|
| E1F1B1C3 | SEQ ID NO: | SEQ ID NO: 2, | SEQ ID NO: | SEQ ID NO: 7, | SEQ ID NO: | SEQ ID NO: 5, | SEQ ID NO: | SEQ ID NO: 8, |
| | 1 | X1 = N, X2 = G | 3 | X1 = N, X2 = G | 4 | X = N | 6 | X = N |
| E1F1B1C3- | SEQ ID NO: | SEQ ID NO: 2, | SEQ ID NO: | SEQ ID NO: 7, | SEQ ID NO: | SEQ ID NO: 5, | SEQ ID NO: | SEQ ID NO: 8, |
| CDRV1 | 1 | X1 = N, X2 = A | 3 | X1 = N, X2 = A | 4 | X = A | 6 | X = A |
| E1F1B1C3- | SEQ ID NO: | SEQ ID NO: 2, | SEQ ID NO: | SEQ ID NO: 7, | SEQ ID NO: | SEQ ID NO: 5, | SEQ ID NO: | SEQ ID NO: 8, |
| CDRV2 | 1 | X1 = A, X2 = G | 3 | X1 = A, X2 = G | 4 | X = A | 6 | X = A |
| E1H10B7C7 | SEQ ID NO: 9, | SEQ ID NO: 10, | SEQ ID NO: | SEQ ID NO: 15, | SEQ ID NO: | SEQ ID NO: | SEQ ID NO: | SEQ ID NO: 16, |
| | X = Y | X1 = N, X2 = G | 11 | X1 = N, X2 = G | 12 | 13 | 14 | X = N |
| E1H10B7- | SEQ ID NO: 9, | SEQ ID NO: 10, | SEQ ID NO: | SEQ ID NO: 15, | SEQ ID NO: | SEQ ID NO: | SEQ ID NO: | SEQ ID NO: 16, |
| CDRV1 | X = Y | X1 = N, X2 = A | 11 | X1 = N, X2 = A | 12 | 13 | 14 | X = S |
| E1H10B7- | SEQ ID NO: 9, | SEQ ID NO: 10, | SEQ ID NO: | SEQ ID NO: 15, | SEQ ID NO: | SEQ ID NO: | SEQ ID NO: | SEQ ID NO: 16, |
| CDRV2 | X = Y | X1 = A, X2 = G | 11 | X1 = A, X2 = G | 12 | 13 | 14 | X = S |
| E1D5E1F1 | SEQ ID NO: 9, | SEQ ID NO: 10, | SEQ ID NO: | SEQ ID NO: | SEQ ID NO: | SEQ ID NO: | SEQ ID NO: | SEQ ID NO: |
| | X = F | X1 = N, X2 = G | 11 | 17 | 12 | 13 | 14 | 18 |

Accordingly, in one embodiment, an antibody of the disclosure, or an antigen binding portion thereof, may comprise:
(a) a heavy chain variable region which may comprise an amino acid sequence listed above in Table 1; and
(b) a light chain variable region which may comprise an amino acid sequence listed above in Table 1, or the V_{L} of another Anti-SIRPα antibody, wherein the antibody specifically binds human SIRPα.

In another embodiment, an antibody of the disclosure, or an antigen binding portion thereof, may comprise:
(a) the CDR1, CDR2, and CDR3 regions of the heavy chain variable region listed above in Table 1; and
(b) the CDR1, CDR2, and CDR3 regions of the light chain variable region listed above in Table 1 or the CDRs of another anti-SIRPα antibody, wherein the antibody specifically binds human SIRPα.

In yet another embodiment, the antibody, or antigen binding portion thereof, includes the heavy chain variable CDR2 region of anti-SIRPα antibody combined with CDRs of other antibodies which bind human SIRPα, e.g., CDR1 and/or CDR3 from the heavy chain variable region, and/or CDR1, CDR2, and/or CDR3 from the light chain variable region of a different anti-SIRPα antibody.

In addition, it is well known in the art that the CDR3 domain, independently from the CDR1 and/or CDR2 domain(s), alone can determine the binding specificity of an antibody for a cognate antigen and that multiple antibodies can predictably be generated having the same binding specificity based on a common CDR3 sequence. See, e.g., Klimka et al., British J. of Cancer 83(2):252-260 (2000); Beiboer et al., J. Mol. Biol. 296:833-849 (2000); Rader et al., Proc. Natl. Acad. Sci. U.S.A. 95:8910-8915 (1998); Barbas et al., J. Am. Chem. Soc. 116:2161-2162 (1994); Barbas et al., Proc. Natl. Acad. Sci. U.S.A. 92:2529-2533 (1995); Ditzel et al., J. Immunol. 157:739-749 (1996); Berezov et al., BIAjournal 8: Scientific Review 8 (2001); Igarashi et al., J. Biochem (Tokyo) 117:452-7 (1995); Bourgeois et al., J. Virol 72:807-10 (1998); Levi et al., Proc. Natl. Acad. Sci. U.S.A. 90:4374-8 (1993); Polymenis and Stoller, J. Immunol. 152:5218-5329 (1994) and Xu and Davis, Immunity 13:37-45 (2000). See also, U.S. Pat. Nos. 6,951,646; 6,914,128; 6,090,382; 6,818,216; 6,156,313; 6,827,925; 5,833,943; 5,762,905 and 5,760,185. Each of these references is hereby incorporated by reference in its entirety.

Accordingly, in another embodiment, antibodies of the disclosure may comprise the CDR2 of the heavy chain variable region of the anti-SIRPα antibody and at least the CDR3 of the heavy and/or light chain variable region of the anti-SIRPα antibody, or the CDR3 of the heavy and/or light chain variable region of another anti-SIRPα antibody, wherein the antibody is capable of specifically binding to human SIRPα. These antibodies preferably (a) compete for binding with SIRPα; (b) retain the functional characteristics; (c) bind to the same epitope; and/or (d) have a similar binding affinity as the anti-SIRPα antibody of the present disclosure. In yet another embodiment, the antibodies further may comprise the CDR2 of the light chain variable region of the anti-SIRPα antibody, or the CDR2 of the light chain variable region of another anti-SIRPα antibody, wherein the antibody is capable of specifically binding to human SIRPα. In another embodiment, the antibodies of the disclosure may include the CDR1 of the heavy and/or light chain variable region of the anti-SIRPα antibody, or the CDR1 of the heavy and/or light chain variable region of another anti-SIRPα antibody, wherein the antibody is capable of specifically binding to human SIRPα.

In another embodiment, an antibody of the disclosure may comprise a heavy and/or light chain variable region sequences of CDR1, CDR2 and CDR3 sequences which differ from those of the anti-SIRPα antibodies of the present disclosure by one or more conservative modifications. It is understood in the art that certain conservative sequence modification can be made which do not remove antigen binding. See, e.g., Brummell et al., (1993) Biochem 32:1180-8; de Wildt et al., (1997) Prot. Eng. 10:835-41; Komissarov et al., (1997) J. Biol. Chem. 272:26864-26870; Hall et al., (1992) J. Immunol. 149:1605-12; Kelley and O'Connell (1993) Biochem. 32:6862-35; Adib-Conquy et al., (1998) Int. Immunol. 10:341-6 and Beers et al., (2000) Clin. Can. Res. 6:2835-43.

Accordingly, in one embodiment, the antibody may comprise a heavy chain variable region which may comprise CDR1, CDR2, and CDR3 sequences and/or a light chain variable region which may comprise CDR1, CDR2, and CDR3 sequences, wherein:
(a) the heavy chain variable region CDR1 sequence may comprise a sequence listed in Table 1 above, and/or conservative modifications thereof; and/or
(b) the heavy chain variable region CDR2 sequence may comprise a sequence listed in Table 1 above, and/or conservative modifications thereof; and/or
(c) the heavy chain variable region CDR3 sequence may comprise a sequence listed in Table 1 above, and conservative modifications thereof; and/or
(d) the light chain variable region CDR1, and/or CDR2, and/or CDR3 sequences may comprise the sequence(s) listed in Table 1 above; and/or conservative modifications thereof; and
(e) the antibody specifically binds human SIRPα.

The antibody of the present disclosure possesses one or more of the following functional properties described above, such as high affinity binding to human SIRPα, and blocking activity on SIRPα-PD-L1 binding.

In various embodiments, the antibody can be, for example, a human, humanized or chimeric antibody.

As used herein, the term “conservative sequence modifications” is intended to refer to amino acid modifications that do not significantly affect or alter the binding characteristics of the antibody containing the amino acid sequence. Such conservative modifications include amino acid substitutions, additions and deletions. Modifications can be introduced into an antibody of the disclosure by standard techniques known in the art, such as site-directed mutagenesis and PCR-mediated mutagenesis. Conservative amino acid substitutions are ones in which the amino acid residue is replaced with an amino acid residue having a similar side chain. Families of amino acid residues having similar side chains have been defined in the art.

These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, one or more amino acid residues within the CDR regions of an antibody of the disclosure can be replaced with other amino acid residues from the same side chain family and the altered antibody can be tested for retained function (i.e., the functions set forth above) using the functional assays described herein.

Antibodies of the disclosure can be prepared using an antibody having one or more of the V_{H}/V_{L} sequences of the anti-SIRPα antibody of the present disclosure as starting material to engineer a modified antibody. An antibody can be engineered by modifying one or more residues within one or both variable regions (i.e., V_{H} and/or V_{L}), for example within one or more CDR regions and/or within one or more framework regions. Additionally or alternatively, an antibody can be engineered by modifying residues within the constant region(s), for example to alter the effector function(s) of the antibody.

In certain embodiments, CDR grafting can be used to engineer variable regions of antibodies. Antibodies interact with target antigens predominantly through amino acid residues that are located in the six heavy and light chain complementarity determining regions (CDRs).

For this reason, the amino acid sequences within CDRs are more diverse between individual antibodies than sequences outside of CDRs. Because CDR sequences are responsible for most antibody-antigen interactions, it is possible to express recombinant antibodies that mimic the properties of specific naturally occurring antibodies by constructing expression vectors that include CDR sequences from the specific naturally occurring antibody grafted onto framework sequences from a different antibody with different properties (see, e.g., Riechmann et al., (1998) Nature 332:323-327; Jones et al., (1986) Nature 321:522-525; Queen et al., (1989) Proc. Natl. Acad. See also U.S.A. 86:10029-10033; U.S. Pat. Nos. 5,225,539; 5,530,101; 5,585,089; 5,693,762 and 6,180,370).

Accordingly, another embodiment of the disclosure pertains to an isolated monoclonal antibody, or antigen binding portion thereof, which may comprise a heavy chain variable region that may comprise CDR1, CDR2, and CDR3 sequences which may comprise the sequences of the present disclosure, as described above, and/or a light chain variable region which may comprise CDR1, CDR2, and CDR3 sequences which may comprise the sequences of the present disclosure, as described above. While these antibodies contain the V_{H} and V_{L} CDR sequences of the monoclonal antibody of the present disclosure, they can contain different framework sequences.

Such framework sequences can be obtained from public DNA databases or published references that include germline antibody gene sequences. For example, germline DNA sequences for human heavy and light chain variable region genes can be found in the “VBase” human germline sequence database (available on the Internet at www.mrc-cpe.cam.ac.uk/vbase), as well as in Kabat et al., (1991), cited supra; Tomlinson et al., (1992) J. Mol. Biol. 227:776-798; and Cox et al., (1994) Eur. J. Immunol. 24:827-836; the contents of each of which are expressly incorporated herein by reference. As another example, the germline DNA sequences for human heavy and light chain variable region genes can be found in the Genbank database. For example, the following heavy chain germline sequences found in the HCo7 HuMAb mouse are available in the accompanying Genbank Accession Nos.: 1-69 (NG-0010109, NT-024637 & BC070333), 3-33 (NG-0010109 & NT-024637) and 3-7 (NG-0010109 & NT-024637). As another example, the following heavy chain germline sequences found in the HCo12 HuMAb mouse are available in the accompanying Genbank Accession Nos.: 1-69 (NG-0010109, NT-024637 & BC070333), 5-51 (NG-0010109 & NT-024637), 4-34 (NG-0010109 & NT-024637), 3-30.3 (CAJ556644) & 3-23 (AJ406678).

Antibody protein sequences are compared against a compiled protein sequence database using one of the sequence similarity searching methods called the Gapped BLAST (Altschul et al., (1997), supra), which is well known to those skilled in the art.

Preferred framework sequences for use in the antibodies of the disclosure are those that are structurally similar to the framework sequences used by antibodies of the disclosure.

The V_{H} CDR1, CDR2, and CDR3 sequences can be grafted onto framework regions that have the identical sequence as that found in the germline immunoglobulin gene from which the framework sequence derives, or the CDR sequences can be grafted onto framework regions that contain one or more mutations as compared to the germline sequences. For example, it has been found that in certain instances it is beneficial to mutate residues within the framework regions to maintain or enhance the antigen binding ability of the antibody (see e.g., U.S. Pat. Nos. 5,530,101; 5,585,089; 5,693,762 and 6,180,370).

Another type of variable region modification is to mutate amino acid residues within the V_{H} and/or V_{L} CDR1, CDR2 and/or CDR3 regions to thereby improve one or more binding properties (e.g., affinity) of the antibody of interest. Site-directed mutagenesis or PCR-mediated mutagenesis can be performed to introduce the mutation(s) and the effect on antibody binding, or other functional property of interest, can be evaluated in in vitro or in vivo assays as known in the art. Preferably conservative modifications (as known in the art) are introduced.

The mutations can be amino acid substitutions, additions or deletions, but are preferably substitutions. Moreover, typically no more than one, two, three, four or five residues within a CDR region are altered.

Accordingly, in another embodiment, the disclosure provides isolated anti-SIRPα monoclonal antibodies, or antigen binding portions thereof, which may comprise a heavy chain variable region that may comprise: (a) a V_{H} CDR1 region which may comprise the sequence of the present disclosure, or an amino acid sequence having one, two, three, four or five amino acid substitutions, deletions or additions; (b) a V_{H} CDR2 region which may comprise the sequence of the present disclosure, or an amino acid sequence having one, two, three, four or five amino acid substitutions, deletions or additions; (c) a V_{H} CDR3 region which may comprise the sequence of the present disclosure, or an amino acid sequence having one, two, three, four or five amino acid substitutions, deletions or additions; (d) a V_{L} CDR1 region which may comprise the sequence of the present disclosure, or an amino acid sequence having one, two, three, four or five amino acid substitutions, deletions or additions; (e) a V_{L} CDR2 region which may comprise the sequence of the present disclosure, or an amino acid sequence having one, two, three, four or five amino acid substitutions, deletions or additions; and (t) a V_{L} CDR3 region which may comprise the sequence of the present disclosure, or an amino acid sequence having one, two, three, four or five amino acid substitutions, deletions or additions.

Engineered antibodies of the disclosure include those in which modifications have been made to framework residues within V_{H} and/or V_{L}, e.g. to improve the properties of the antibody. Typically, such framework modifications are made to decrease the immunogenicity of the antibody. For example, one approach is to “back-mutate” one or more framework residues to the corresponding germline sequence. More specifically, an antibody that has undergone somatic mutation can contain framework residues that differ from the germline sequence from which the antibody is derived. Such residues can be identified by comparing the antibody framework sequences to the germline sequences from which the antibody is derived.

Another type of framework modification involves mutating one or more residues within the framework region, or even within one or more CDR regions, to remove T cell epitopes to thereby reduce the potential immunogenicity of the antibody. This approach is also referred to as “de-immunization” and is described in further detail in U.S. Patent Publication No. 20030153043.

In addition, or as an alternative to modifications made within the framework or CDR regions, antibodies of the disclosure can be engineered to include modifications within the Fc region, typically to alter one or more functional properties of the antibody, such as serum half-life, complement fixation, Fc receptor binding, and/or antigen-dependent cellular cytotoxicity.

Furthermore, an antibody of the disclosure can be chemically modified (e.g., one or more chemical moieties can be attached to the antibody) or be modified to alter its glycosylation, again to alter one or more functional properties of the antibody.

In one embodiment, the hinge region of C_{H1} is modified in such that the number of cysteine residues in the hinge region is altered, e.g., increased or decreased. This approach is described further in U.S. Pat. No. 5,677,425. The number of cysteine residues in the hinge region of C_{H1} is altered to, for example, facilitate assembly of the light and heavy chains or to increase or decrease the stability of the antibody.

In another embodiment, the Fc hinge region of an antibody is mutated to decrease the biological half-life of the antibody. More specifically, one or more amino acid mutations are introduced into the C_{H2}-C_{H3} domain interface region of the Fc-hinge fragment such that the antibody has impaired Staphylococcyl protein A (SpA) binding relative to native Fc-hinge domain SpA binding. This approach is described in further detail in U.S. Pat. No. 6,165,745.

In still another embodiment, the glycosylation of an antibody is modified. For example, a glycosylated antibody can be made (i.e., the antibody lacks glycosylation). Glycosylation can be altered to, for example, increase the affinity of the antibody for antigen. Such carbohydrate modifications can be accomplished by, for example, altering one or more sites of glycosylation within the antibody sequence. For example, one or more amino acid substitutions can be made that result in elimination of one or more variable region framework glycosylation sites to thereby eliminate glycosylation at that site. Such aglycosylation may increase the affinity of the antibody for antigen. See, e.g., U.S. Pat. Nos. 5,714,350 and 6,350,861.

Additionally or alternatively, an antibody can be made that has an altered type of glycosylation, such as a hypo fucosylated antibody having reduced amounts of fucosyl residues or an antibody having increased bisecting GlcNac structures. Such altered glycosylation patterns have been demonstrated to increase or reduce the ADCC ability of antibodies. Such carbohydrate modifications can be accomplished by, for example, expressing the antibody in a host cell with altered glycosylation machinery. Cells with altered glycosylation machinery have been described in the art and can be used as host cells in which to express recombinant antibodies of the disclosure to thereby produce an antibody with altered glycosylation. For example, the cell lines Ms704, Ms705, and Ms709 lack the fucosyltransferase gene, FUT8 (α(1,6)-fucosyltransferase), such that antibodies expressed in the Ms704, Ms705, and Ms709 cell lines lack fucose on their carbohydrates. The Ms704, Ms705, and Ms709 FUT8-/- cell lines were created by the targeted disruption of the FUT8 gene in CHO/DG44 cells using two replacement vectors (see U.S. Patent Publication No. 20040110704 and Yamane-Ohnuki et al., (2004) Biotechnol Bioeng 87:614-22). As another example, EP 1,176,195 describes a cell line with a functionally disrupted FUT8 gene, which encodes a fucosyl transferase, such that antibodies expressed in such a cell line exhibit hypofucosylation by reducing or eliminating the α-1,6 bond-related enzyme. EP 1,176,195 also describes cell lines which have a low enzyme activity for adding fucose to the N-acetylglucosamine that binds to the Fc region of the antibody or does not have the enzyme activity, for example the rat myeloma cell line YB2/0 (ATCC CRL 1662). PCT Publication WO 03/035835 describes a variant CHO cell line, Lec13 cells, with reduced ability to attach fucose to Asn (297)-linked carbohydrates, also resulting in hypofucosylation of antibodies expressed in that host cell (see also Shields et al., (2002) J. Biol. Chem. 277:26733-26740). Antibodies with a modified glycosylation profile can also be produced in chicken eggs, as described in PCT Publication WO 06/089231. Alternatively, antibodies with a modified glycosylation profile can be produced in plant cells, such as Lemna.

Methods for production of antibodies in a plant system are disclosed in the U.S. patent application corresponding to Alston & Bird LLP attorney docket No. 040989/314911, filed on Aug. 11, 2006. The fucose residues of the antibody can be cleaved off using a fucosidase enzyme; e.g., the fucosidase α-L-fucosidase removes fucosyl residues from antibodies (Tarentino et al., (1975) Biochem. 14:5516-23).

Another modification of the antibodies herein that is contemplated by this disclosure is pegylation. An antibody can be pegylated to, for example, increase the biological (e.g., serum) half-life of the antibody. To pegylate an antibody, the antibody, or fragment thereof, typically is reacted with polyethylene glycol (PEG), such as a reactive ester or aldehyde derivative of PEG, under conditions in which one or more PEG groups become attached to the antibody or antibody fragment. Preferably, the pegylation is carried out via an acylation reaction or an alkylation reaction with a reactive PEG molecule (or an analogous reactive water-soluble polymer). As used herein, the term “polyethylene glycol” is intended to encompass any of the forms of PEG that have been used to derivatize other proteins, such as mono (C₁-C₁₀) alkoxy- or aryloxy-polyethylene glycol or polyethylene glycol-maleimide. In certain embodiments, the antibody to be pegylated is an aglycosylated antibody. Methods for pegylating proteins are known in the art and can be applied to the antibodies of the disclosure. See, e.g., EP 0 154 316 and EP 0 401 384.

Antibodies of the disclosure can be characterized by their various physical properties, to detect and/or differentiate different classes thereof.

For example, antibodies can contain one or more glycosylation sites in either the light or heavy chain variable region. Such glycosylation sites may result in increased immunogenicity of the antibody or an alteration of the pK of the antibody due to altered antigen binding (Marshall et al (1972) Annu Rev Biochem 41:673-702; Gala and Morrison (2004) J Immunol 172:5489-94; Wallick et al (1988) J Exp Med 168:1099-109; Spiro (2002) Glycobiology 12:43R-56R; Parekh et al (1985) Nature 316:452-7; Mimura et al., (2000) Mol Immunol 37:697-706). Glycosylation has been known to occur at motifs containing an N-X-S/T sequence. In some instances, it is preferred to have an anti-SIRPα antibody that does not contain variable region glycosylation. This can be achieved either by selecting antibodies that do not contain the glycosylation motif in the variable region or by mutating residues within the glycosylation region.

In a preferred embodiment, the antibodies do not contain asparagine isomerism sites. The deamidation of asparagine may occur on N-G or D-G sequences and result in the creation of an isoaspartic acid residue that introduces a link into the polypeptide chain and decreases its stability (isoaspartic acid effect).

Each antibody will have a unique isoelectric point (pI), which generally falls in the pH range between 6 and 9.5. The pI for an IgG1 antibody typically falls within the pH range of 7-9.5 and the pI for an IgG4 antibody typically falls within the pH range of 6-8. There is speculation that antibodies with a pI outside the normal range may have some unfolding and instability under in vivo conditions. Thus, it is preferred to have an anti-SIRPα antibody that contains a pI value that falls in the normal range. This can be achieved either by selecting antibodies with a pI in the normal range or by mutating charged surface residues.

In another aspect, the disclosure provides nucleic acid molecules that encode heavy and/or light chain variable regions, or CDRs, of the antibodies of the disclosure. The nucleic acids can be present in whole cells, in a cell lysate, or in a partially purified or substantially pure form. A nucleic acid is “isolated” or “rendered substantially pure” when purified away from other cellular components or other contaminants, e.g., other cellular nucleic acids or proteins, by standard techniques. A nucleic acid of the disclosure can be, e.g., DNA or RNA and may or may not contain intronic sequences. In a preferred embodiment, the nucleic acid is a cDNA molecule.

Nucleic acids of the disclosure can be obtained using standard molecular biology techniques. For antibodies expressed by hybridomas (e.g., hybridomas prepared from transgenic mice carrying human immunoglobulin genes as described further below), cDNAs encoding the light and heavy chains of the antibody made by the hybridoma can be obtained by standard PCR amplification or cDNA cloning techniques. For antibodies obtained from an immunoglobulin gene library (e.g., using phage display techniques), a nucleic acid encoding such antibodies can be recovered from the gene library.

Preferred nucleic acids molecules of the disclosure include those encoding the V_{H} and V_{L} sequences of the SIRPα monoclonal antibody or the CDRs. Once DNA fragments encoding V_{H} and V_{L} segments are obtained, these DNA fragments can be further manipulated by standard recombinant DNA techniques, for example to convert the variable region genes to full-length antibody chain genes, to Fab fragment genes or to a scFv gene. In these manipulations, a V_{L}- or V_{H}-encoding DNA fragment is operatively linked to another DNA fragment encoding another protein, such as an antibody constant region or a flexible linker.

The term “operatively linked”, as used in this context, is intended to mean that the two DNA fragments are joined such that the amino acid sequences encoded by the two DNA fragments remain in-frame.

The isolated DNA encoding the V_{H} region can be converted to a full-length heavy chain gene by operatively linking the V_{H}-encoding DNA to another DNA molecule encoding heavy chain constant regions (C_{H1}, C_{H2} and C_{H3}). The sequences of human heavy chain constant region genes are known in the art and DNA fragments encompassing these regions can be obtained by standard PCR amplification. The heavy chain constant region can be an IgG1, IgG2, IgG3, IgG4, IgA, IgE, IgM or IgD constant region, but most preferably is an IgG1 or IgG4 constant region. For a Fab fragment heavy chain gene, the V_{H}-encoding DNA can be operatively linked to another DNA molecule encoding only the heavy chain C_{H1} constant region.

The isolated DNA encoding the V_{L} region can be converted to a full-length light chain gene (as well as a Fab light chain gene) by operatively linking the V_{L}-encoding DNA to another DNA molecule encoding the light chain constant region, CL. The sequences of human light chain constant region genes are known in the art and DNA fragments encompassing these regions can be obtained by standard PCR amplification. In preferred embodiments, the light chain constant region can be a kappa or lambda constant region.

To create a scFv gene, the V_{H}- and V_{L}-encoding DNA fragments are operatively linked to another fragment encoding a flexible linker, e.g., encoding the amino acid sequence (Gly4-Ser)3, such that the V_{H} and V_{L} sequences can be expressed as a contiguous single-chain protein, with the V_{L} and V_{H} regions joined by the flexible linker (see e.g., Bird et al., (1988) Science 242:423-426; Huston et al., (1988) Proc. Natl. Acad. Sci. USA 85:5879-5883; McCafferty et al., (1990) Nature 348:552-554).

Monoclonal antibodies (mAbs) of the present disclosure can be produced using the well-known somatic cell hybridization (hybridoma) technique of Kohler and Milstein (1975) Nature 256: 495. Other embodiments for producing monoclonal antibodies include viral or oncogenic transformation of B lymphocytes and phage display techniques. Chimeric or humanized antibodies are also well known in the art. See e.g., U.S. Pat. Nos. 4,816,567; 5,225,539; 5,530,101; 5,585,089; 5,693,762 and 6,180,370, the contents of which are specifically incorporated herein by reference in their entirety.

Antibodies of the disclosure also can be produced in a host cell transfectoma using, for example, a combination of recombinant DNA techniques and gene transfection methods as is well known in the art (e.g., Morrison, S. (1985) Science 229:1202). In one embodiment, DNA encoding partial or full-length light and heavy chains obtained by standard molecular biology techniques is inserted into one or more expression vectors such that the genes are operatively linked to transcriptional and translational regulatory sequences. In this context, the term “operatively linked” is intended to mean that an antibody gene is ligated into a vector such that transcriptional and translational control sequences within the vector serve their intended function of regulating the transcription and translation of the antibody gene.

The term “regulatory sequence” is intended to include promoters, enhancers and other expression control elements (e.g., polyadenylation signals) that control the transcription or translation of the antibody genes. Such regulatory sequences are described, e.g., in Goeddel (Gene Expression Technology. Methods in Enzymology 185, Academic Press, San Diego, Calif. (1990)). Preferred regulatory sequences for mammalian host cell expression include viral elements that direct high levels of protein expression in mammalian cells, such as promoters and/or enhancers derived from cytomegalovirus (CMV), Simian Virus 40 (SV40), adenovirus, e.g., the adenovirus major late promoter (AdMLP) and polyomavirus enhancer.

Alternatively, non-viral regulatory sequences can be used, such as the ubiquitin promoter or β-globin promoter. Still further, regulatory elements composed of sequences from different sources, such as the SRa promoter system, which contains sequences from the SV40 early promoter and the long terminal repeat of human T cell leukemia virus type 1 (Takebe et al., (1988) Mol. Cell. Biol. 8:466-472). The expression vector and expression control sequences are chosen to be compatible with the expression host cell used.

The antibody light chain gene and the antibody heavy chain gene can be inserted into the same or separate expression vectors. In preferred embodiments, the variable regions are used to create full-length antibody genes of any antibody isotype by inserting them into expression vectors already encoding heavy chain constant and light chain constant regions of the desired isotype such that the V_{H} segment is operatively linked to the CH segment(s) within the vector and the V_{L} segment is operatively linked to the CL segment within the vector. Additionally or alternatively, the recombinant expression vector can encode a signal peptide that facilitates secretion of the antibody chain from a host cell. The antibody chain gene can be cloned into the vector such that the signal peptide is linked in-frame to the amino terminus of the antibody chain gene. The signal peptide can be an immunoglobulin signal peptide or a heterologous signal peptide (i.e., a signal peptide from a non-immunoglobulin protein).

In addition to the antibody chain genes and regulatory sequences, the recombinant expression vectors of the disclosure can carry additional sequences, such as sequences that regulate replication of the vector in host cells (e.g., origins of replication) and selectable marker genes. The selectable marker gene facilitates selection of host cells into which the vector has been introduced (see, e.g., U.S. Pat. Nos. 4,399,216; 4,634,665 and 5,179,017). For example, typically the selectable marker gene confers resistance to drugs, such as G418, hygromycin or methotrexate, on a host cell into which the vector has been introduced. Preferred selectable marker genes include the dihydrofolate reductase (DHFR) gene (for use in dhfr-host cells with methotrexate selection/amplification) and the neo gene (for G418 selection).

For expression of the light and heavy chains, the expression vector(s) encoding the heavy and light chains is transfected into a host cell by standard techniques. The various forms of the term “transfection” are intended to encompass a wide variety of techniques commonly used for the introduction of exogenous DNA into a prokaryotic or eukaryotic host cell, e.g., electroporation, calcium-phosphate precipitation, DEAE-dextran transfection and the like.

Although it is theoretically possible to express the antibodies of the disclosure in either prokaryotic or eukaryotic host cells, expression of antibodies in eukaryotic cells, and most preferably mammalian host cells, is the most preferred because such eukaryotic cells, and in particular mammalian cells, are more likely than prokaryotic cells to assemble and secrete a properly folded and immunologically active antibody.

Preferred mammalian host cells for expressing the recombinant antibodies of the disclosure include Chinese Hamster Ovary (CHO cells) (including dhfr-CHO cells, described in Urlaub and Chasin, (1980) Proc. Natl. Acad. Sci. USA 77:4216-4220, used with a DHFR selectable marker, e.g., as described in R. J. Kaufman and P. A. Sharp (1982) J. Mol. Biol. 159:601-621), NSO myeloma cells, COS cells and SP2 cells. In particular for use with NSO myeloma cells, another preferred expression system is the GS gene expression system disclosed in WO 87/04462, WO 89/01036 and EP 338,841. When recombinant expression vectors encoding antibody genes are introduced into mammalian host cells, the antibodies are produced by culturing the host cells for a period of time sufficient to allow for expression of the antibody in the host cells or, more preferably, secretion of the antibody into the culture medium in which the host cells are grown. Antibodies can be recovered from the culture medium using standard protein purification methods.

Antibodies or antigen-binding portions thereof of the disclosure can be conjugated to a therapeutic agent to form an immunoconjugate such as an antibody-drug conjugate (ADC).

Suitable therapeutic agents include cytotoxins, alkylating agents, DNA minor groove binders, DNA intercalators, DNA crosslinkers, histone deacetylase inhibitors, nuclear export inhibitors, proteasome inhibitors, topoisomerase I or II inhibitors, heat shock protein inhibitors, tyrosine kinase inhibitors, antibiotics, and anti-mitotic agents. In the ADC, the antibody and therapeutic agent preferably are conjugated via a linker cleavable such as a peptidyl, disulfide, or hydrazone linker. More preferably, the linker is a peptidyl linker such as Val-Cit, Ala-Val, Val-Ala-Val, Lys-Lys, Pro-Val-Gly-Val-Val, Ala-Asn-Val, Val-Leu-Lys, Ala-Ala-Asn, Cit-Cit, Val-Lys, Lys, Cit, Ser, or Glu. The ADCs can be prepared as described in U.S. Pat. Nos. 7,087,600; 6,989,452; and 7,129,261; PCT Publications WO 02/096910; WO 07/038,658; WO 07/051,081; WO 07/059,404; WO 08/083,312; and WO 08/103,693; U.S. Patent Publications 20060024317; 20060004081; and 20060247295; the disclosures of which are incorporated herein by reference.

In another aspect, the present disclosure features bispecific molecules which may comprise one or more antibodies of the disclosure linked to at least one other functional molecule, e.g., another peptide or protein (e.g., another antibody or ligand for a receptor) to generate a bispecific molecule that binds to at least two different binding sites or target molecules. Thus, as used herein, “bispecific molecule” includes molecules that have three or more specificities.

In an embodiment, a bispecific molecule has, in addition to the FcR binding specificity and an anti-SIRPα binding specificity, a third specificity. The third specificity can be for a tumor associated antigen such as CD19, CD20, CD22, CD4, CD24, CD38, CD123, CD228, CD138, BCMA, GPC3, CEA, folate receptor (FRα), mesothelin, CD276, gp100, 5T4, GD2, EGFR, MUC-1, PSMA, EpCAM, MCSP, SM5-1, MICA, MICB, ULBP and HER-2.

Bispecific molecules may be in many different formats and sizes. At one end of the size spectrum, a bispecific molecule retains the traditional antibody format, except that, instead of having two binding arms of identical specificity, it has two binding arms each having a different specificity. At the other extreme are bispecific molecules consisting of two single-chain antibody fragments (scFv's) linked by a peptide chain, a so-called Bs(scFv)2 construct.

Intermediate-sized bispecific molecules include two different F(ab) fragments linked by a peptidyl linker. Bispecific molecules of these and other formats can be prepared by genetic engineering, somatic hybridization, or chemical methods. See, e.g., Kufer et al, cited supra; Cao and Suresh, Bioconjugate Chemistry, 9 (6), 635-644 (1998); and van Spriel et al., Immunology Today, 21 (8), 391-397 (2000), and the references cited therein.

An oncolytic virus preferentially infects and kills cancer cells. Antibodies of the present disclosure can be used in conjunction with oncolytic viruses. Alternatively, oncolytic viruses encoding antibodies of the present invention can be introduced into human body.

Also provided herein are a chimeric antigen receptor (CAR) containing an anti-SIRPα scFv, the anti-SIRPα scFv may comprise CDRs and heavy/light chain variable regions described herein.

The anti-SIRPα CAR may comprise (a) an extracellular antigen binding domain which may comprise an anti-SIRPα scFv; (b) a transmembrane domain; and (c) an intracellular signaling domain.

The CAR may contain a signal peptide at the N-terminus of the extracellular antigen binding domain that directs the nascent receptor into the endoplasmic reticulum, and a hinge peptide at the N-terminus of the extracellular antigen binding domain that makes the receptor more available for binding. The CAR preferably comprises, at the intracellular signaling domain, a primary intracellular signaling domain and one or more co-stimulatory signaling domains. The mainly used and most effective primary intracellular signaling domain is CD3-zeta cytoplasmic domain which contains ITAMs, the phosphorylation of which results in T cell activation. The co-stimulatory signaling domain may be derived from the co-stimulatory proteins such as CD28, CD137 and OX40.

The CARs may further add factors that enhance T cell expansion, persistence, and anti-tumor activity, such as cytokines, and co-stimulatory ligands.

Also provided are engineered immune effector cells, which may comprise the CAR provided herein. In certain embodiments, the immune effector cell is a T cell, an NK cell, a peripheral blood mononuclear cell (PBMC), a hematopoietic stem cell, a pluripotent stem cell, or an embryonic stem cell. In certain embodiments, the immune effector cell is a T cell.

In another aspect, the present disclosure provides a pharmaceutical composition which may comprise one or more antibodies (or antigen-binding portion thereof, or the bispecifics, immune cells with CARs, immunoconjugates, oncolytic viruses, or nucleic acid molecules or expression vectors expressing the same) of the present disclosure formulated together with a pharmaceutically acceptable carrier. The antibodies (or antigen-binding portion thereof, or the bispecifics, immune cells with CARs, immunoconjugates, oncolytic viruses, or nucleic acid molecules or expression vectors expressing the same) can be dosed separately when the composition contains more than one antibody (or antigen-binding portion thereof, or the bispecifics, immune cells with CARs, immunoconjugates, oncolytic viruses, or nucleic acid molecules or expression vectors expressing the same). The composition may optionally contain one or more additional pharmaceutically active ingredients, such as another antibody or a drug, such as an anti-tumor drug.

The pharmaceutical composition may comprise any number of excipients. Excipients that can be used include carriers, surface active agents, thickening or emulsifying agents, solid binders, dispersion or suspension aids, solubilizers, colorants, flavoring agents, coatings, disintegrating agents, lubricants, sweeteners, preservatives, isotonic agents, and combinations thereof. The selection and use of suitable excipients are taught in Gennaro, ed., Remington: The Science and Practice of Pharmacy, 20th Ed. (Lippincott Williams & Wilkins 2003), the disclosure of which is incorporated herein by reference.

Preferably, the pharmaceutical composition is suitable for intravenous, intramuscular, subcutaneous, parenteral, spinal or epidermal administration (e.g., by injection or infusion). Depending on the route of administration, the active ingredient can be coated in a material to protect it from the action of acids and other natural conditions that may inactivate it. The phrase “parenteral administration” as used herein means modes of administration other than enteral and topical administration, usually by injection, and includes, without limitation, intravenous, intramuscular, intra-arterial, intrathecal, intracapsular, intraorbital, intracardiac, intradermal, intraperitoneal, transtracheal, subcutaneous, subcuticular, intra-articular, subcapsular, subarachnoid, intraspinal, epidural and intrasternal injection and infusion. Alternatively, an antibody of the disclosure can be administered via a non-parenteral route, such as a topical, epidermal or mucosal route of administration, e.g., intranasally, orally, vaginally, rectally, sublingually or topically.

Pharmaceutical compositions can be in the form of sterile aqueous solutions or dispersions. They can also be formulated in a microemulsion, liposome, or other ordered structure suitable to high drug concentration.

The amount of active ingredient which can be combined with a carrier material to produce a single dosage form will vary depending upon the subject being treated and the particular mode of administration and will generally be that amount of the composition which produces a therapeutic effect. Generally, out of one hundred percent, this amount will range from about 0.01% to about ninety-nine percent of active ingredient, preferably from about 0.1% to about 70%, most preferably from about 1% to about 30% of active ingredient in combination with a pharmaceutically acceptable carrier.

Dosage regimens are adjusted to provide the optimum desired response (e.g., a therapeutic response). For example, a single bolus can be administered, several divided doses can be administered over time or the dose can be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active ingredient calculated to produce the desired therapeutic effect in association with the required pharmaceutical carrier. Alternatively, antibody can be administered as a sustained release formulation, in which case less frequent administration is required.

For administration of the composition, the dosage may range from about 0.0001 to 100 mg/kg. An exemplary treatment regime entails administration once per week.

A “therapeutically effective dosage” of an anti-SIRPα antibody, or the antigen-binding portion thereof, or an bispecifics, an immune cell with CAR, an immunoconjugate, an oncolytic virus, or a nucleic acid molecule or an expression vector expressing the same, of the disclosure preferably results in a decrease in severity of disease symptoms, an increase in frequency and duration of disease symptom-free periods, or a prevention of impairment or disability due to the disease affliction. For example, for the treatment of tumor-bearing subjects, a “therapeutically effective dosage” preferably eliminate inflammations by at least about 20%, more preferably by at least about 40%, even more preferably by at least about 60%, and still more preferably by at least about 80% relative to untreated subjects.

The pharmaceutical composition can be a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. See, e.g., Sustained and Controlled Release Drug Delivery Systems, J. R. Robinson, ed., Marcel Dekker, Inc., New York, 1978.

Therapeutic compositions can be administered via medical devices such as (1) needleless hypodermic injection devices (e.g., U.S. Pat. Nos. 5,399,163; 5,383,851; 5,312,335; 5,064,413; 4,941,880; 4,790,824; and 4,596,556); (2) micro-infusion pumps (U.S. Pat. No. 4,487,603); (3) transdermal devices (U.S. Pat. No. 4,486,194); (4) infusion apparatuses (U.S. Pat. Nos. 4,447,233 and 4,447,224); and (5) osmotic devices (U.S. Pat. Nos. 4,439,196 and 4,475,196); the disclosures of which are incorporated herein by reference.

In certain embodiments, the monoclonal antibodies or antigen binding portions thereof of the disclosure can be formulated to ensure proper distribution in vivo. For example, to ensure that the therapeutic antibody of the disclosure cross the blood-brain barrier, they can be formulated in liposomes, which may additionally comprise targeting moieties to enhance selective transport to specific cells or organs. See, e.g. U.S. Pat. Nos. 4,522,811; 5,374,548; 5,416,016; and 5,399,331; V. V. Ranade (1989) J. Clin. Pharmacol. 29:685; Umezawa et al., (1988) Biochem. Biophys. Res. Commun. 153:1038; Bloeman et al., (1995) FEBS Lett. 357:140; M. Owais et al., (1995) Antimicrob. Agents Chemother. 39:180; Briscoe et al., (1995) Am. J. Physiol. 1233:134; Schreier et al., (1994) J. Biol. Chem. 269:9090; Keinanen and Laukkanen (1994) FEBS Lett. 346:123; and Killion and Fidler (1994) Immunomethods 4:273.

The pharmaceutical composition which may comprise the antibodies or the antigen-binding portion thereof, or the bispecifics, immune cells with CARs, immunoconjugates, oncolytic viruses, or nucleic acid molecules or expression vectors expressing the same, of the present disclosure have numerous in vitro and in vivo utilities involving, for example, treatment of cancers.

The disclosure provides a method for treating cancers associated with SIRPα signaling, which may comprise administering to a subject a therapeutically effective amount of the composition of the present disclosure.

The cancers can be solid or hematologic cancers, including, but not limited to, non-small lung cancer, breast cancer, ovarian cancer, renal cell cancer, colorectal cancer and pancreatic cancer. The composition comprises the antibody, or the antigen-binding portion thereof, with a weak or strong FcR binding heavy chain constant region, the bispecific molecule, the immunoconjugate, the immune cell with CAR, the oncolytic virus, the nucleic acid molecule, or the expression vector of the disclosure. If the composition comprises the antibody or the antigen binding portion thereof with strong FcR binding affinity, the immunoconjugate, the immune cell with CAR, or the oncolytic virus, then local delivery of the composition to the tumor site(s) is preferred. The antibodies of the present disclosure can be, for example, chimeric or human antibodies. In certain embodiments, the subject is human. In some embodiments, at least one additional anti-cancer antibody can be further administered, such as an antibody targeting a tumor associated antigen such as CD19, CD20, CD22, CD4, CD24, CD38, CD123, CD228, CD138, BCMA, GPC3, CEA, folate receptor (FRα), mesothelin, CD276, gp100, 5T4, GD2, EGFR, MUC-1, PSMA, EpCAM, MCSP, SM5-1, MICA, MICB, ULBP, and HER-2, or an antibody targeting an inhibitory immune checkpoint, such as LAG-3, PD-1, VISTA, or CTLA-4. In yet another embodiment, an antibody, or an antigen-binding portion thereof, of the disclosure is administered with a cytokine (e.g., IL-2 and/or IL-21), or a costimulatory antibody (e.g., an anti-CD137 and/or anti-GITR antibody). In another embodiment, an antibody, or an antigen-binding portion thereof, of the disclosure is administered with a chemotherapeutic agent, which may be a cytotoxic agent, such as epirubicin, oxaliplatin, and/or 5-fluorouracil (5-FU).

In yet another aspect, the disclosure provides a method of modulating or enhancing an immune response in a subject comprising administering to the subject the composition of the disclosure such that the immune response in the subject is modulated/enhanced. The composition comprises the antibody, or the antigen-binding portion thereof, with weak FcR binding heavy chain constant regions, the bispecific molecule, the nucleic acid molecule, or the expression vector of the disclosure. The antibodies of the present disclosure can be, for example, chimeric or human antibodies. In certain embodiments, the subject is human.

In another aspect, the disclosure provides methods of combination therapy in which the pharmaceutical composition of the present disclosure is co-administered with one or more additional antibodies that are effective in inhibiting tumor growth in a subject. In one embodiment, the disclosure provides a method for inhibiting tumor growth in a subject which may comprise administering to the subject the pharmaceutical composition of the disclosure and one or more additional antibodies, such as an antibody targeting a tumor associated antigen such as CD19, CD20, CD22, CD4, CD24, CD38, CD123, CD228, CD138, BCMA, GPC3, CEA, folate receptor (FRα), mesothelin, CD276, gp100, 5T4, GD2, EGFR, MUC-1, PSMA, EpCAM, MCSP, SM5-1, MICA, MICB, ULBP, and HER-2, or an antibody targeting an inhibitory immune checkpoint, such as LAG-3, PD-1, VISTA, or CTLA-4. The SIRPα pathway blockade may also be further combined with standard cancer treatments. For example, SIRPα pathway blockade can be combined with chemotherapeutic regimes. For example, a chemotherapeutic agent can be administered with the anti-SIRPα antibodies, which may be a cytotoxic agent. For example, epirubicin, oxaliplatin, and 5-FU are administered to patients receiving anti-SIRPα therapy. Optionally, the combination of anti-SIRPα and one or more additional antibodies can be further combined with an immunogenic agent, such as cancerous cells, purified tumor antigens (including recombinant proteins, peptides, and carbohydrate molecules), and cells transfected with genes encoding immune stimulating cytokines (He et al., (2004) J. Immunol. 173:4919-28). Non-limiting examples of tumor vaccines that can be used include peptides of melanoma antigens, such as peptides of gp100, MAGE antigens, Trp-2, MART1 and/or tyrosinase, or tumor cells transfected to express the cytokine GM-CSF. Other therapies that may be combined with anti-SIRPα antibody includes, but not limited to, interleukin-2 (IL-2) administration, radiation, surgery, or hormone deprivation.

The combination of therapeutic agents discussed herein can be administered concurrently as a single composition in a pharmaceutically acceptable carrier, or concurrently as separate compositions with each agent in a pharmaceutically acceptable carrier. In another embodiment, the combination of therapeutic agents can be administered sequentially.

Furthermore, if more than one dose of the combination therapy is administered sequentially, the order of the sequential administration can be reversed or kept in the same order at each time point of administration, sequential administrations can be combined with concurrent administrations, or any combination thereof.

Although the present invention and its advantages have been described in detail, it should be understood that various changes, substitutions and alterations can be made herein without departing from the spirit and scope of the invention as defined in the appended claims.

The present disclosure is further illustrated by the following examples, which should not be construed as further limiting. The contents of all figures and all references, Genbank sequences, patents and published patent applications cited throughout this application are expressly incorporated herein by reference.

### EXAMPLES

### Example 1. Generation of Anti-SIRPα Monoclonal Antibodies Using Hybridoma Technology

### Immunization

AceMouse™ mice (AceMab Ltd., Hunan Province, China), genetically engineered to produce antibodies with human heavy and light chain variable regions and mouse constant regions, were immunized according to the method as described in E Harlow, D. Lane, Antibody: A Laboratory Manual, Cold Spring Harbor Laboratory Press, Cold Spring Harbor, N.Y., 1998.

The recombinant human SIRPα-Fc protein (in house made with SEQ ID NO: 21) was used as the immunogen, and also used for determining anti-sera titers and for screening hybridomas secreting antigen-specific antibodies.

Immunizing dosages contained 22.5 μg recombinant human SIRPα-Fc proteins per mouse per injection for primary and boost immunizations. To increase immune response, the complete Freud's adjuvant and incomplete Freud's adjuvant (Sigma, St. Louis, Mo., USA) were used respectively for primary and boost immunizations. Briefly, the antigen was prepared in PBS or saline with the concentration ranging from 0.23 to 0.3 mg/ml, the calculated amount of antigen was then added to the desired amount of adjuvant, and the resulting mixture was mixed by gently vortexing for 2 min to generate a water-in-oil emulsion. The adjuvant-antigen emulsion was then drawn into the proper syringe for animal injection. A total of 22.5 μg of antigen was injected in a volume of 150-200 μl. Each animal was immunized, and then boosted for 3 to 4 times depending on the anti-sera titer. Animals with good titers as determined by ELISA were given a final boost by intraperitoneal injection before fusion.

### Hybridoma Fusion and Screening

Cells of murine myeloma cell line (SP2/0-Ag14, ATCC #CRL-1581) were cultured to reach the log phase stage right before fusion. Spleen cells from immunized mice were prepared sterilely and fused with murine myeloma cells according to the method as described in Kohler G, and Milstein C, “Continuous cultures of fused cells secreting antibody of predefined specificity,” Nature, 256: 495-497 (1975). Fused “hybrid cells” were subsequently dispensed into 96-well plates in DMEM/20% FCS/HAT medium. Surviving hybridoma colonies were observed under the microscope seven to ten days post fusion. After two weeks, the supernatant from each well was subjected to Capture ELISA using in house made biotin-labeled human SIRPα-Fc protein. Positive hybridomas secreting antibodies that bound to the human SIRPα-Fc protein were selected and transferred to 24-well plates. These hybridomas were further tested for their activity for blocking human SIRPα binding to cell surface human CD47.

Hybridoma clones producing antibodies that showed high specific human SIRPα-Fc binding and human SIRPα-cell surface human CD47 blocking activity were subcloned by limited dilution to ensure the clonality of the cell line, and then monoclonal antibodies were purified. Briefly, Protein A sepharose columns (from bestchrom (Shanghai) Biosciences, Cat #AA0273) were washed using PBS buffer in 5 to 10 column volumes. Cell supernatants of monoclonal hybridomas were passed through the columns, and then the columns were washed using PBS buffer until the absorbance for protein reached the baseline. The columns were eluted with elution buffer (0.1 M Glycine-HCl, pH 2.7), and immediately collected into tubes with neutralizing buffer (1 M Tris-HCl, pH 9.0). Fractions containing immunoglobulins were pooled and dialyzed in PBS overnight at 4° C.

### Example 2. Binding Activity of Anti-SIRPα Monoclonal Antibodies

The purified anti-SIRPα monoclonal antibodies, including E1D5E1F1, E1F1B1C3, and E1H10B7C7, generated in Example 1 were characterized for their binding activity to human SIRPα, cynomolgus SIRPα and human SIRPβ by Capture ELISA, Indirect ELISA and Flow Cytometry (FACS). KWAR23 (described in “Ring N G, et al. Anti-SIRPα antibody immunotherapy enhances neutrophil and macrophage antitumor activity. Proc Natl Acad Sci USA. 2017 Dec. 5; 114(49): E10578-E10585”, referred to as BM herein, in house made with the heavy chain and light chain amino acid sequences set forth in SEQ ID NOs: 32 and 33, respectively), and BI-765063 (Boehringer Ingelheim International GmbH, referred to as BM5 herein, in house made with the heavy chain and light chain amino acid sequences set forth in SEQ ID NOs: 34 and 35, respectively) were used as positive controls.

### 2.1 Capture ELISA

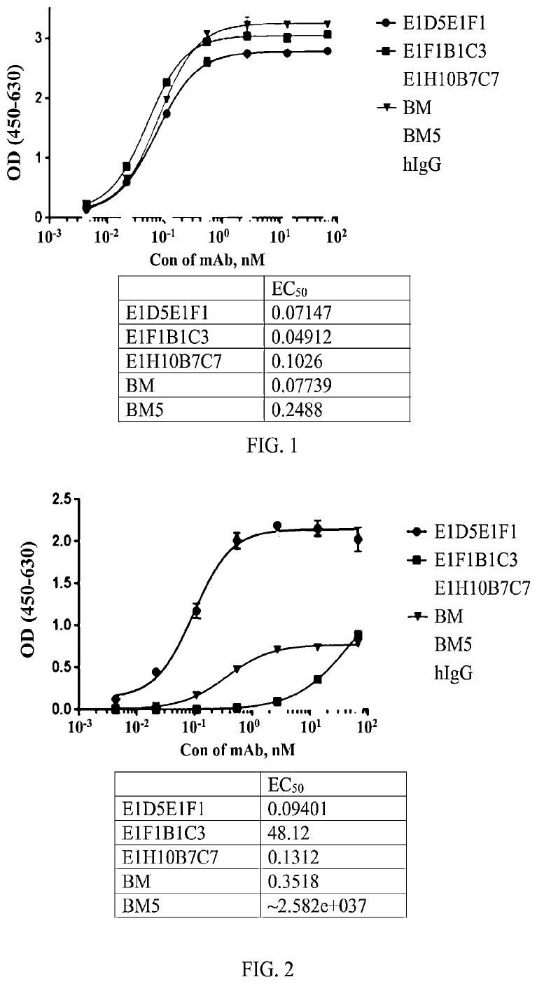

Briefly, 96-well plates were coated with 100 μl 2 μg/ml AffiniPure Goat Anti-Mouse IgG, F(ab′)2 fragment specific (Jackson ImmunoResearch, Cat #115-005-072) in PBS at 37° C. for 2 h. The plates were washed once with wash buffer (PBS+0.05% Tween-20, PBST) and then blocked with 200 μl blocking buffer (5% w/v non-fatty milk in PBST) overnight at 4° C. The plates were washed again and incubated respectively with 100 μl serially diluted anti-SIRPα antibodies of the disclosure, the positive controls and the negative control (hIgG, human immunoglobulin (pH4) for intravenous injection, Hualan Biological Engineering Inc.) (5-fold dilution in 2.5% non-fatty milk in PBST, starting at 66.7 nM) for 40 min at 37° C., and then washed 4 times again. The plates containing the captured anti-SIRPα antibodies were incubated with 100 μl biotin-labeled human SIRPα-Fc protein (in house made with SEQ ID NO: 21, 3.8 ng/mL in 2.5% non-fatty milk in PBST) for 40 min at 37° C., washed 4 times, and incubated with streptavidin conjugated HRP (1:10000 dilution in PBST, Jackson Immuno Research, Cat #016-030-084, 100 μl/well) for 40 min at 37° C. After a final wash, the plates were incubated with 100 μl TMB (Innoreagents, Cat #TMB-S-002) at room temperature. The reaction was stopped in 3 min with 50 μl/well 1 M H2SO₄, and the absorbance of each well was read on a microplate reader using the dual wavelength mode with 450 nm for TMB and 630 nm as the reference wavelength. The OD (450-630) values were plotted against the antibody concentration. Data was analyzed using Graphpad Prism and EC₅₀ values were reported. The results were shown in FIG. 1.

### 2.2 Indirect ELISA

Briefly, 96-well micro plates were coated with 100 μl 2 μg/ml cynomolgus SIRPα-his protein (in house made with SEQ ID NO: 25), or 2 μg/ml human SIRPα-isoform 2-his protein (in house made with SEQ ID NO: 28) in carbonate/bicarbonate buffer (pH 9.6) for 2 h at 37° C.

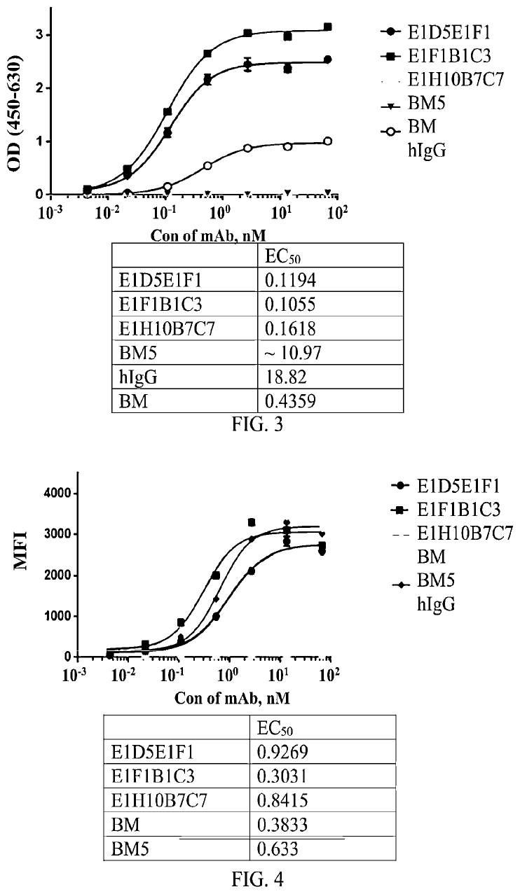

The plates were washed once with wash buffer (PBS+0.05% Tween-20, PBST) and then blocked with 200 μl/well blocking buffer (5% w/v non-fatty milk in PBST) overnight at 4° C. The plates were washed again and incubated with 100 μl serially diluted anti-SIRPα antibodies of the disclosure or the controls (starting at 66.7 nM, 5-fold serial dilution in 2.5% non-fatty milk in PBST) for 40 min at 37° C. The plates were washed 4 times and incubated with Peroxidase AffiniPure Goat Anti-Mouse IgG, Fcγ Fragment Specific (1:5000 dilution in PBST buffer, Jackson Immunoresearch, Cat #115-035-071, 100 μl/well) for 40 min at 37° C. After a final wash, the plates were incubated with 100 μl TMB (Innoreagents) at room temperature. The reaction was stopped 3-10 min later with 50 μl 1 M H2SO₄, and the absorbance of each well was read on a microplate reader using the dual wavelength mode with 450 nm for TMB and 630 nm as the reference wavelength. The OD (450-630) values were plotted against the antibody concentration. Data was analyzed using Graphpad Prism and EC₅₀ values were reported. The results were shown in FIG. 2 and FIG. 3.

### 2.3 Cell Based Binding FACS

The binding activity of anti-SIRPα antibodies of the disclosure to cell surface human SIRPα, human SIRPβ or human SIRPγ were tested by flow cytometry (FACS), using Biosion in-house prepared HEK293-SIRPα cells (expressing full length human SIRPα on cell membranes, uniprot #P78324), HEK293-SIRPβ cells (expressing full length human SIRPβ on cell membranes, uniprot #000241) and Jurkat cells purchased from ATCC (endogenously expressing full length human SIRPγ on cell membranes). The HEK293-SIRPα cells and HEK293-SIRPβ cells were prepared by transfecting HEK293 cells with pCMV-T-P plasmids inserted with human SIRPα and human SIRPβ coding sequences respectively following the instruction of lipofectamine 3000 transfection reagent (Thermo Fisher). The cells were harvested from the cell culture flasks, washed twice and suspended in PBS containing 2% v/v Fetal Bovine Serum (FACS buffer). Then, 1×10⁵ cells per well in 96 well-plates were incubated with 100 μl serially diluted anti-SIRPα antibodies of the disclosure or the controls (starting from 10 μg/mL, 5-fold serial dilution) in FACS buffer for 50 min on ice. Cells were washed twice with FACS buffer, and added with 100 μl R-Phycoerythrin AffiniPure F(ab′)2 Fragment Goat Anti-Mouse IgG (H+L) (1:1000 dilution in FACS buffer, Jackson Immuno Research, Cat #115-116-146). Following an incubation of 50 min at 4° C. in dark, cells were washed three times and re-suspended in FACS buffer. Fluorescence was measured using a Becton Dickinson FACS Canto II-HTS equipment, and the MFI (mean fluorescence intensity) was plotted against the antibody concentration. Data was analyzed using Graphpad Prism and EC₅₀ values were reported. The results were shown in FIGS. 4-6.

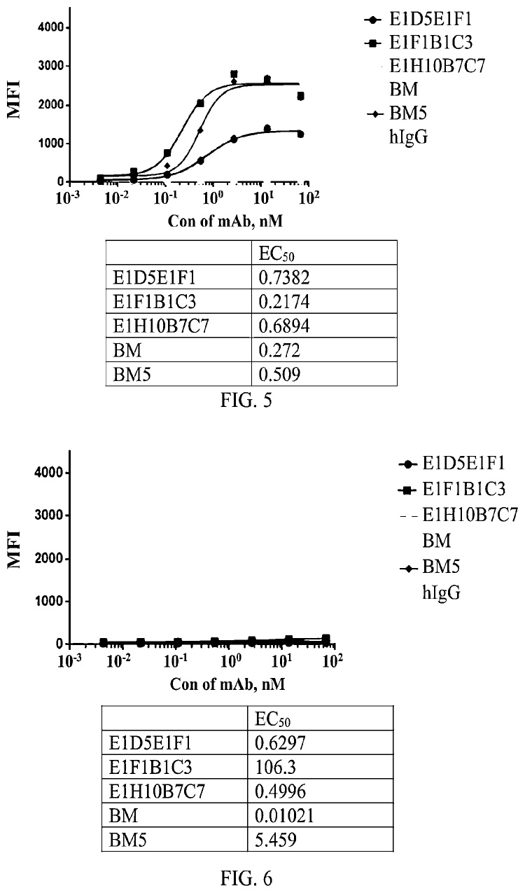

It can be seen from FIG. 1 and FIG. 3 that the antibodies E1D5E1F1, E1F1B1C3 and E1H10B7C7 specifically bound to human SIRPα, and cross-reacted with human SIRPα-isoform 2 with higher Bmax (maximal binding) and lower EC₅₀ than the positive controls. Distinct from BM and BM5, the antibodies E1D5E1F1 and E1H10B7C7 showed high binding activity to cynomolgus SIRPα (FIG. 2). Particularly, the antibody E1F1B1C3 exhibited high binding activity to both cells expressing human SIRPα (FIG. 4) and cells expressing human SIRPβ (FIG. 5) in the cell based binding FACS test. According to FIG. 6, the antibodies E1D5E1F1, E1F1B1C3 and E1H10B7C7 did not bind to human SIRPγ.

### Example 3. Blocking Activity of Anti-SIRPα Antibody on SIRPα-CD47 Interaction

### 3.1 Cell Based Ligand-Blocking FACS

The capability of the anti-SIRPα antibodies of the disclosure to block the binding of the CD47 protein to the cell surface SIRPα was evaluated by FACS, using in house made 293F-SIRPα cells. Briefly, 293F cells were transfected with pCMV-T-P plasmid constructs with the nucleotide sequence encoding human SIRPα (NP_542970.1) between EcoRI and XbaI, following the instruction of lipofectamine 3000 transfection reagent (Thermo Fisher).

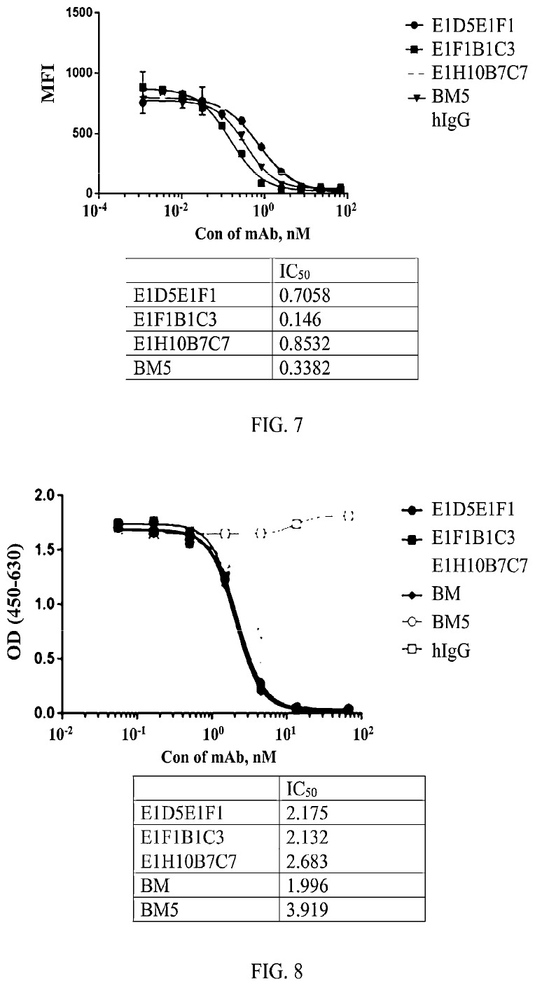

The 293F-SIRPα cells were harvested from cell culture flasks, washed twice and re-suspended in PBS containing 2% v/v Fetal Bovine Serum (FACS buffer). Then, 1×10⁵ cells per well in 96 well-plates were incubated with 100 μl serially diluted anti-SIRPα antibodies of the disclosure or the controls (starting from 10 μg/mL, 5-fold serial dilution in FACS buffer) for 50 min at 4° C. After washed twice in FACS buffer, 100 μl per well of the biotin-labeled human CD47-Fc protein (prepared in-house with SEQ ID NO: 31, 148 ng/mL) in FACS buffer was added to the plates, and the plates were incubated at 4° C. for 60 min. The plates were washed twice with FACS buffer, and then added and incubated for 40 min at 4° C. in dark with 100 μl R-Phycoerythrin Streptavidin (1:500 dilution in FACS buffer, Jackson Immunoresearch, Cat #016-110-084). Cells were washed twice and re-suspended in FACS buffer. Fluorescence was measured using a Becton Dickinson FACS Canto II-HTS equipment, and the MFI (mean fluorescence intensity) was plotted against the antibody concentration. Data was analyzed using Graphpad Prism and IC₅₀ values were reported. The results were shown in FIG. 7.

### 3.2 Ligand Blocking ELISA

The ability of the anti-SIRPα antibodies to block human SIRPα-CD47 binding was also measured in a competitive ELISA assay. Briefly, 100 μl human SIRPα-his protein (in house made with SEQ ID NO: 22) at 2 μg/mL in PBS was coated on 96-well micro plates for 2 h at 37° C. The plates were washed once with wash buffer (PBS+0.05% Tween-20, PBST) and then blocked with 200 μl blocking buffer (5% w/v non-fatty milk in PBST) for 2 h at 37° C. While blocking, the anti-SIRPα antibodies of the disclosure or the controls were diluted with 2.5% non-fatty milk in PBST, starting at 10 μg/ml with a 5-fold serial dilution, and added to the plates, 100 μl per well, and incubated for 40 min at 37° C. After plate washing for three times, 0.8 mg/ml biotin labeled human CD47-his proteins (SEQ ID NO: 30) were 1:5000 diluted with 2.5% milk, and the resulting solutions were added to the plates, 100 μl per well. After incubation at 37° C. for 40 min, the plates were washed using wash buffer, added with 100 μl streptavidin conjugated HRP, and incubated for 40 min at 37° C. to detect the biotin-labeled human CD47-his bound to the SIRPα. The plates were washed again using wash buffer and added with TMB, the reaction was stopped using 1 M H2SO₄. The absorbance of each well was read on a microplate reader using the dual wavelength mode with 450 nm for TMB and 630 nm as the reference wavelength, and the OD (450-630) values were plotted against the antibody concentration. Data was analyzed using Graphpad Prism and IC₅₀ values were reported. The results were shown in FIG. 8.

It can be seen from FIG. 7 that the anti-SIRPα antibodies E1D5E1F1, E1F1B1C3 and E1H10B7C7 were capable of blocking the binding of human CD47 to cell surface human SIRPα, with comparable blocking activity to BM5.

FIG. 8 showed that the anti-SIRPα antibodies E1D5E1F1, E1F1B1C3 and E1H10B7C7 were able to block human SIRPα-CD47 binding, with comparable blocking activity to BM and BM5.

### Example 4. Engineering of Anti-SIRPα Antibodies

The anti-SIRPα antibodies E1F1B1C3 and E1H10B7C7 were engineered in the CDR and framework regions where an asparagine (Asn, N) residue was immediately followed by a glycine residue (Gly, G) such that deamination is likely to occur. In particular, the antibody E1H10B7C7 originally had an N-G residue combination in the heavy chain CDR2 region and an N-G residue combination in the light chain framework 3 region which is also a glycosylation site, and was thus genetically engineered at these two sites to eliminate the deamination and glycosylation risk, as shown in Table 2-1 below. The antibody E1F1B1C3 had two high-risk deamination sites, i.e., an N-G residue combination in the heavy chain CDR2 region, and an N-G residue combination in the light chain CDR2-FR3 region, and was thus engineered at these two sites, as shown in Table 2-2 below. The amino acid sequences of the heavy chain and light chain variable regions after genetic engineering were set forth in Table 1.

TABLE 2-1
Engineered E1H10B7C7 antibodies in hIgG4 format

| mAb | Heavy chain | Light chain | Constant Region |
|---|---|---|---|
| E1H10B7C7 | no mutation | no mutation | hIgG4 (S228P + YTE + K447 Del)/hKappa |
| E1H10B7-CDRV1-IgG4 | G56A | N65S | hIgG4 (S228P + YTE + K447 Del)/hKappa |
| E1H10B7-CDRV2-IgG4 | N55A | N65S | hIgG4 (S228P + YTE + K447 Del)/hKappa |

TABLE 2-2
Engineered E1F1B1C3 antibodies in hIgG4 format

| mAb | Heavy chain | Light chain | Constant Region |
|---|---|---|---|
| E1F1B1C3 | no mutation | no mutation | hIgG4 (S228P + YTE + K447 Del)/hKappa |
| E1F1B1C3-CDRV1-IgG4 | G56A | N56A | hIgG4 (S228P + YTE + K447 Del)/hKappa |
| E1F1B1C3-CDRV2-IgG4 | N55A | N56A | hIgG4 (S228P + YTE + K447 Del)/hKappa |

The engineered antibodies were expressed with human IgG4 (S228P+YTE+K447 Del) constant region (SEQ ID NO: 19) and human kappa constant region (SEQ ID NO: 20), wherein the human IgG4 (S228P+YTE+K447 Del) constant region, compared to the wild-typed one, may reduce the occurrence of arm exchange in IgG4 antibodies and prolong the half-life of the antibodies.

The engineered antibodies were also expressed with the human IgG1 (AQQ+K447 Del or AQQ+YTE+K447 Del) constant region (SEQ ID NO: 38 (X1=M, X2=S, X3=T; or X1=Y, X2=T, X3=E)) and human kappa constant region (SEQ ID NO: 20), as shown in Table 3-1 and 3-2, wherein the IgG1 constant region with the mentioned modifications had enhanced Fey receptor binding affinity (and thus prolonged half-life) and enhanced antibody stability (i.e., decreased antibody heterogeneity).

TABLE 3-1
Engineered E1H10B7C7 antibodies in hIgG1format

| mAb | Heavy chain | Light chain | Constant Region |
|---|---|---|---|
| E1H10B7-CDRV1-IgG1 (YTE) | G56A | N65S | hIgG1 (AQQ + YTE + K447 Del)/hKappa |
| E1H10B7-CDRV1-IgG1 | G56A | N65S | hIgG1 (AQQ + K447 Del)/hKappa |

TABLE 3-2
Engineered E1F1B1C3 antibodies in hIgG1 format

| mAb | Heavy chain | Light chain | Constant Region |
|---|---|---|---|
| E1F1B1C3-CDRV1-IgG1 (YTE) | G56A | N56A | hIgG1 (AQQ + YTE + K447 Del)/hKappa |
| E1F1B1C3-CDRV1-IgG1 | G56A | N56A | hIgG1 (AQQ + K447 Del)/hKappa |

### Example 5. Characterization of Engineered Antibodies

The antibodies prepared in Example 4, namely E1H10B7-CDRV1-IgG1 (YTE), E1H10B7-CDRV1-IgG1, E1F1B1C3-CDRV1-IgG1 (YTE) and E1F1B1C3-CDRV1-IgG1, were purified as described above and tested in BIAcore, indirect ELISA, cell based binding FACS and cell based ligand blocking FACS, following the protocols in the foregoing Examples or set forth below.

### 5.1 Binding Affinity Determination Using BIACORE Surface Plasmon Resonance

The engineered anti-SIRPα antibodies were characterized for their binding affinity and binding kinetics by Biacore T200 system (GE healthcare, Pittsburgh, PA, USA).

Briefly, a Protein A chip (GE healthcare, Cat #Cytiva 29127555) was used. The engineered antibodies of the disclosure or the controls at 2 μg/ml were flowed onto the chip at a flow rate of 10 μL/min. Then, serially diluted human SIRPα-his protein (in house made, amino acid sequence set forth in SEQ ID NO: 22), human SIRPβ-his protein (in house made, amino acid sequence set forth in SEQ ID NO: 23), human SIRPγ-his protein (in house made, amino acid sequence set forth in SEQ ID NO: 24), or cynomolgus SIRPα-his (in house made, amino acid sequence set forth in SEQ ID NO: 25), 2-fold dilution in HBS-EP⁺ buffer (provided by Biacore) starting at 200 nM, were flowed onto the chip at a flow rate of 30 μL/min. The antigen-antibody association kinetics was followed for 2 min and the dissociation kinetics was followed for 10 min. The association and dissociation curves were fit to a 1:1 Langmuir binding model using BIAcore evaluation software. The K_{D}, Kₐ and K_{d} values were determined and summarized in Tables 4-1, and 4-2 below.

TABLE 4-1
Binding affinity of engineered anti-SIRPα antibodies to human SIRPα and human SIRPβ

| | Kinetics on Biacore | | | | | |
|---|---|---|---|---|---|---|
| | Human SIRPα | | | Human SIRPβ | | |
| | Kₐ | K_{d} | K_{D} | Kₐ | K_{d} | K_{D} |
| mAb ID# | (M⁻¹s⁻¹) | (s⁻¹) | (M) | (M⁻¹s⁻¹) | (s⁻¹) | (M) |
| E1H10B7-CDRV1-IgG1 (YTE) | 1.29E+05 | 1.88E−03 | 1.46E−08 | BDL | BDL | BDL |
| E1H10B7-CDRV1-IgG1 | 1.34E+05 | 1.18E−03 | 8.78E−09 | BDL | BDL | BDL |
| E1F1B1C3-CDRV1-IgG1 (YTE) | 1.28E+06 | 1.05E−03 | 8.24E−10 | 7.13E+05 | 2.00E−03 | 2.81E−09 |
| E1F1B1C3-CDRV1-IgG1 | 1.17E+06 | 4.02E−04 | 3.44E−10 | 7.73E+05 | 9.07E−04 | 1.17E−09 |
| BM5 | 2.90E+05 | 7.75E−03 | 2.67E−08 | 3.04E+05 | 9.28E−03 | 3.05E−08 |

BDL: below detection limit

TABLE 4-2
Binding affinity of engineered anti-SIRPα
antibodies to monkey SIRPα

| | monkey SIRPα | | |
|---|---|---|---|
| | Kₐ | K_{d} | K_{D} |
| mAb ID# | (M⁻¹s⁻¹) | (s⁻¹) | (M) |
| E1H10B7-CDRV1-IgG1 (YTE) | 2.64E+04 | 1.46E−02 | 5.53E−07 |
| E1H10B7-CDRV1-IgG1 | 3.50E+04 | 1.32E−02 | 3.76E−07 |
| E1F1B1C3-CDRV1-IgG1 (YTE) | | Low binding | |
| E1F1B1C3-CDRV1-IgG1 | | Low binding | |
| BM5 | | No binding | |

TABLE 4-3
Binding affinity of engineered anti-SIRPα antibodies to FcRn

| | FcRn | |
|---|---|---|
| mAb ID# | K_{D} (M) in pH 6.0 | K_{D} (M) in pH 7.4 |
| E1H10B7-CDRV1-IgG1(YTE) | 2.88E−08 | 1.29E−07 |
| E1H10B7-CDRV1-IgG1 | 9.41E−08 | No binding |
| E1F1B1C3-CDRV1-IgG1(YTE) | 2.68E−08 | 6.51E−07 |
| E1F1B1C3-CDRV1-IgG1 | 5.67E−08 | No binding |

For the binding affinity of engineered anti-SIRPα monoclonal antibodies of the disclosure to human FcRn protein (Cat #FCM-H5286, Acro biosystems Inc.), a CM5 chip (carboxy methyl dextran coated chip from GE healthcare, Cat #BR100530) which was covalently linked with human FcRn protein was used. The engineered anti-SIRPα monoclonal antibodies, 2-fold dilution in HBS-EP⁺ buffer (provided by Biacore) starting at 62.5 μg/ml, were flowed onto the chip at a flow rate of 30 μL/min. The K_{D}, Kₐ and K_{d} values were determined and summarized in Tables 4-3 below.

The BIAcore test data showed that the antibodies E1H10B7-CDRV1-IgG1 (YTE) and E1H10B7-CDRV1-IgG1 specifically bound to human SIRPα but not to human SIRPβ or human SIRPγ (data not shown), while the antibodies E1F1B1C3-CDRV1-IgG1 (YTE) and E1F1B1C3-CDRV1-IgG1 bound to both human SIRPα and human SIRPβ but not to human SIRPγ. Further, the antibodies E1H10B7-CDRV1-IgG1 (YTE) and E1F1B1C3-CDRV1-IgG1 (YTE) showed higher binding affinity to FcRn at pH 6.0 than their respective counterparts without YTE modification, and thus probably had potential longer half-life in human body.

### 5.2 Binding Activity Determination of Engineered Anti-SIRPα Antibodies Using Indirect ELISA, Cell Based Binding FACS and Cell Based Ligand Blocking FACS

The indirect ELISA for testing the antibodies' binding activity to mouse SIRPα-his (in house made with SEQ ID NO: 37, mouse SIRPα (BAA20376.1, Met 1-Phe 363)) was performed following the protocol of Example 2, wherein Peroxidase AffiniPure F(ab′)₂ Fragment Goat Anti-Human IgG, Fey fragment specific (Jackson Immunoresearch, Cat #109-036-098) was used instead of Peroxidase AffiniPure Goat Anti-Mouse IgG, Fcγ Fragment Specific, 100 μl/well.

The anti-SIRPα antibodies of the disclosure were also tested for their cross-reaction with human SIRPα-V8 by indirect ELISA following the protocol of Example 2, wherein the 96-well micro plates were coated with 100 μl 2 μg/ml human SIRPα-V8-his protein (in house made with SEQ ID NO: 29) in carbonate/bicarbonate buffer (pH 9.6).

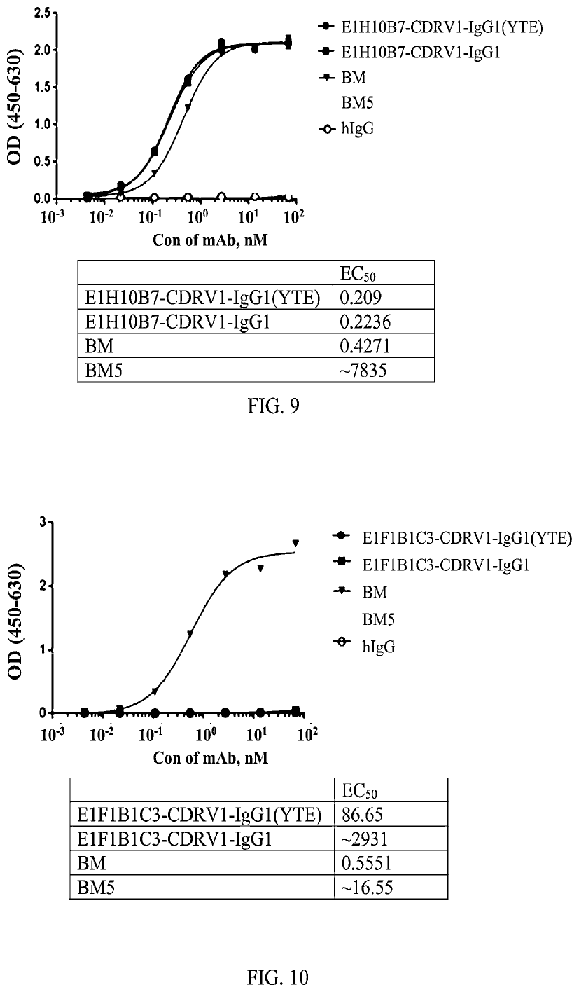

The results of the indirect ELISAs were shown in FIGS. 9-16.

For the cell based binding FACS, R-Phycoerythrin AffiniPure Goat Anti-human IgG Fcγ fragment specific (Jackson Immunoresearch, Cat #109-115-098) was used instead of R-Phycoerythrin AffiniPure F(ab′)2 Fragment Goat Anti-Mouse IgG (H+L), 1:1000 dilution in FACS buffer, 100 μl/well. The binding activity of the anti-SIRPα antibodies to human SIRPα, human SIRPβ and human SIRPγ was also tested by flow cytometry (FACS) following similar protocols. The results of the cell based binding FACS were shown in FIGS. 17-22.

For the cell based ligand-blocking FACS, the capability of the anti-SIRPα antibodies to block binding of CD47 protein to cell surface human SIRPα, and to block binding of SIRPα protein to cell surface human CD47 was evaluated by FACS, using the in house made 293F-SIRPα cells and 293F-CD47 cells, respectively. Briefly, 293F cells were transfected with pCMV-T-P plasmid constructs with the nucleotide sequence encoding human SIRPα (NP_542970.1) or human CD47 (Uniprot #Q08722). The results of the cell based ligand-blocking FACS tests were shown in FIGS. 23-26.

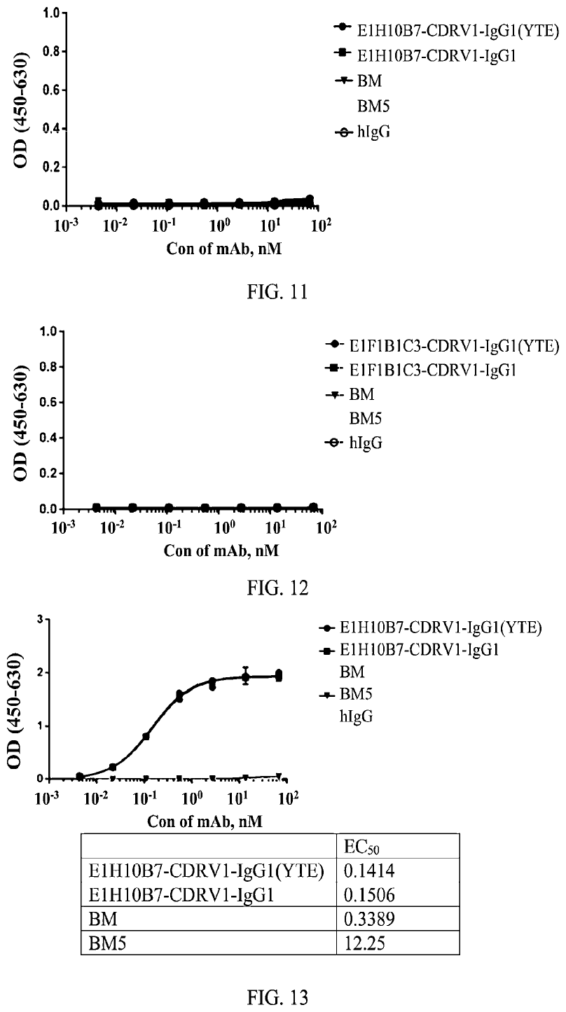

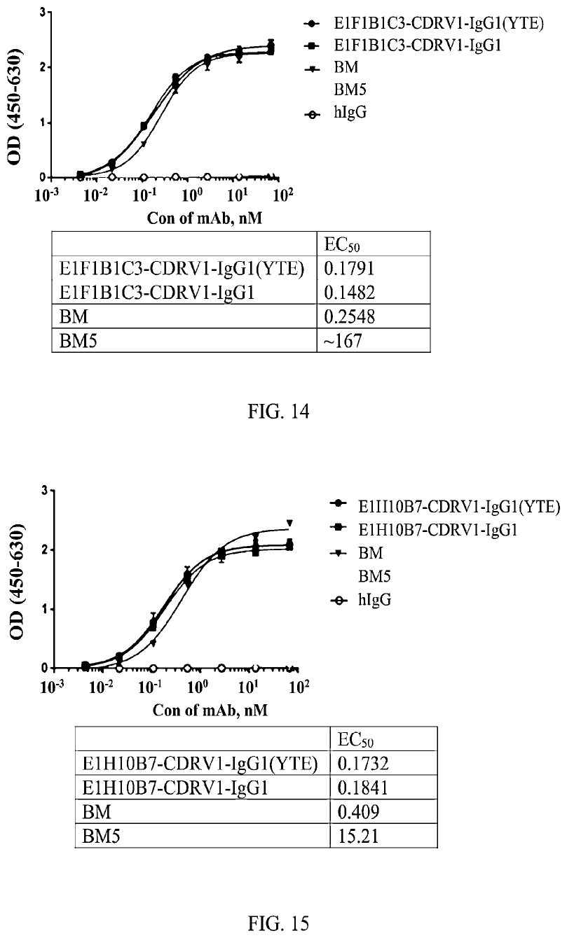

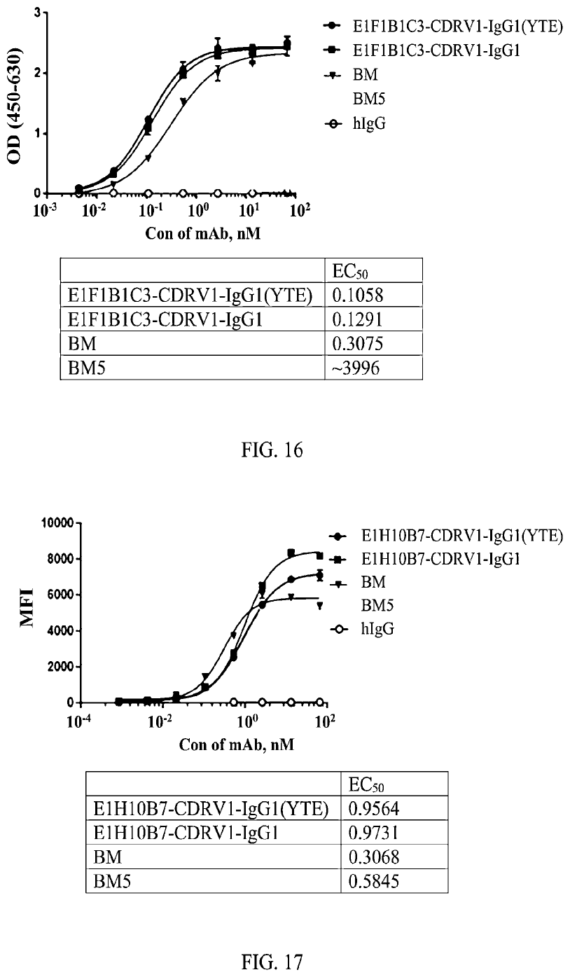

In the indirect ELISA, E1H10B7-CDRV1-IgG1 (YTE), E1H10B7-CDRV1-IgG1, E1F1B1C3-CDRV1-IgG1 (YTE) and E1F1B1C3-CDRV1-IgG1 specifically bound to monkey SIRPα (FIGS. 9-10), human SIRPα-V8 (FIGS. 13-14) and human SIRPα-isoform 2 (FIGS. 15-16), while did not bind mouse SIRPα (FIGS. 11-12).

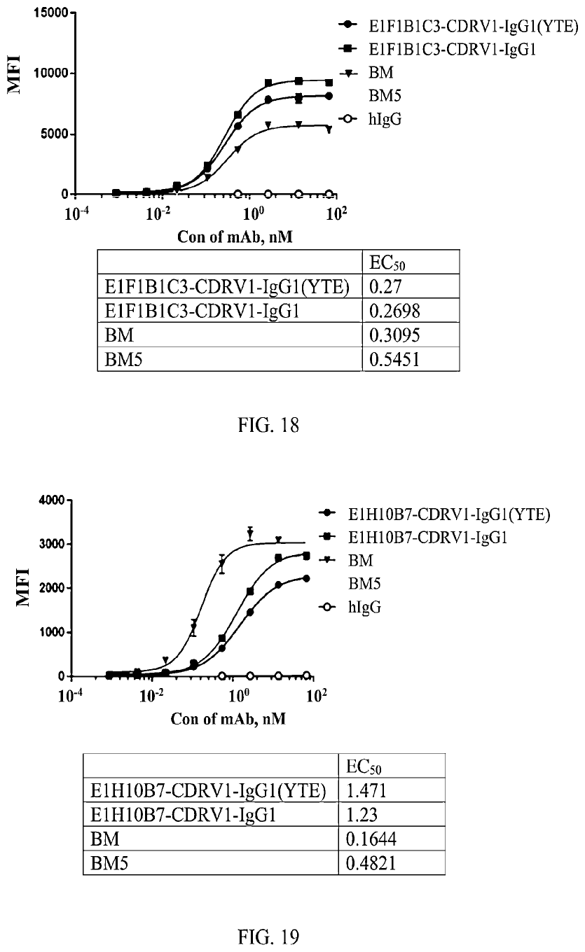

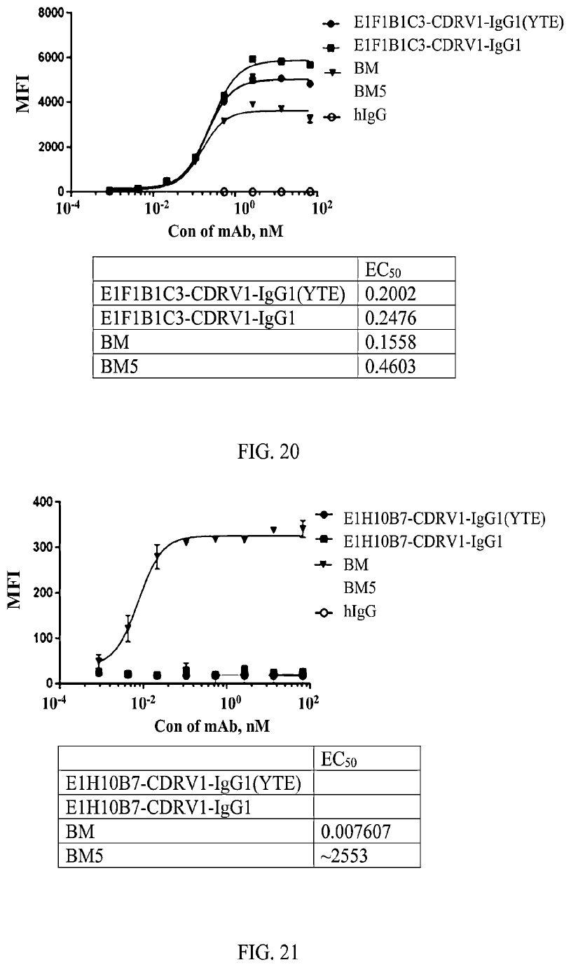

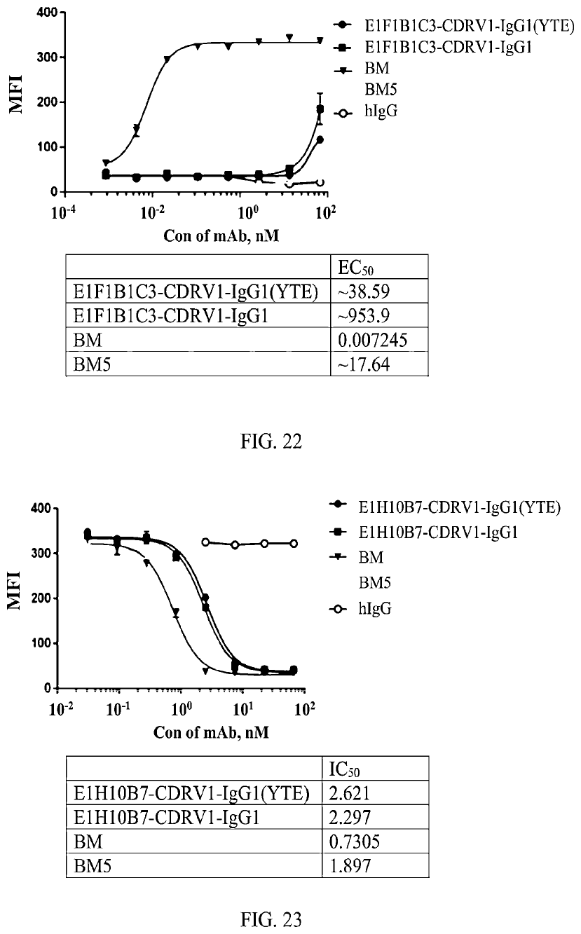

In the cell based binding FACS tests, the data showed that E1H10B7-CDRV1-IgG1 (YTE) and E1H10B7-CDRV1-IgG1 showed comparable binding activity to cell surface human SIRPα (FIG. 17) and weaker binding activity to cell surface human SIRPβ (FIG. 19) when compared to BM and BM5, while E1F1B1C3-CDRV1-IgG1 (YTE) and E1F1B1C3-CDRV1-IgG1 showed higher binding activity to cell surface human SIRPα and human SIRPβ than BM and BM5, as shown in FIG. 18 and FIG. 20. Similar to the BM5, E1H10B7-CDRV1-IgG1 (YTE), E1H10B7-CDRV1-IgG1, E1F1B1C3-CDRV1-IgG1 (YTE) and E1F1B1C3-CDRV1-IgG1 did not bind to human SIRPγ expressed on Jurkat cells, while BM did (FIGS. 21-22).

The antibodies E1H10B7-CDRV1-IgG1 (YTE) and E1H10B7-CDRV1-IgG1 were capable of blocking binding of human CD47 to cell surface human SIRPα (FIG. 23), and binding of human SIRPα to cell surface human CD47 (FIG. 25), at comparable activity to BM5.

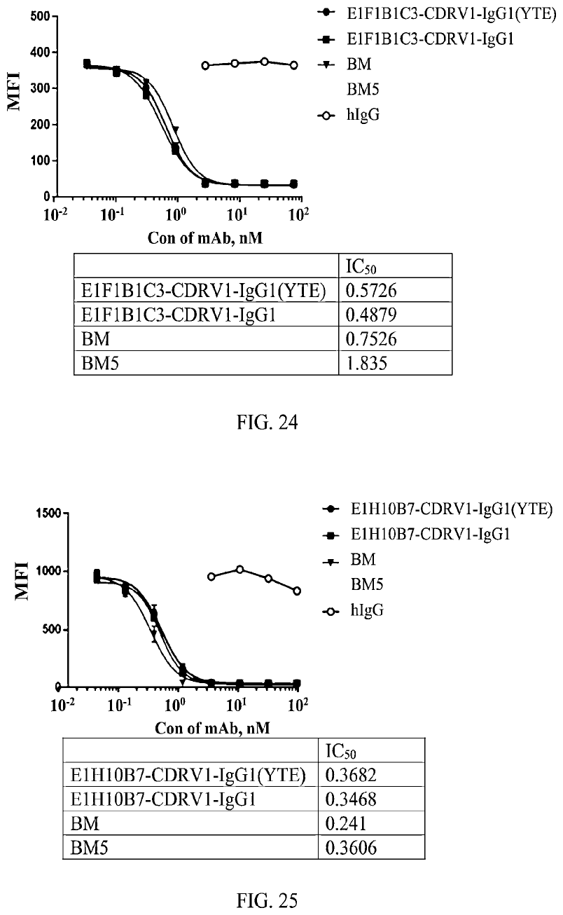

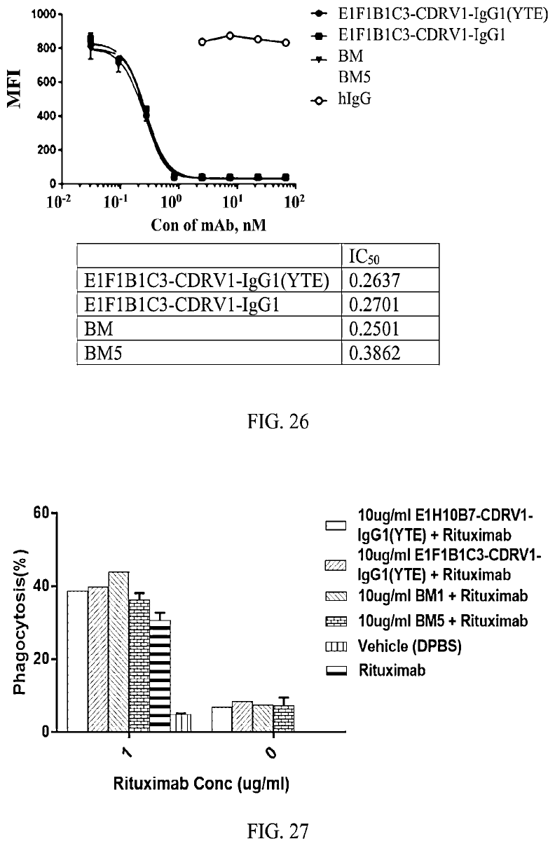

The antibodies E1F1B1C3-CDRV1-IgG1 (YTE) and E1F1B1C3-CDRV1-IgG1 were capable of blocking binding of human CD47 to cell surface human SIRPα (FIG. 24) and binding of human SIRPα to cell surface human CD47 (FIG. 26) at comparable activity to BM. These results indicated that the anti-SIRPα monoclonal antibodies of the disclosure had high SIRPα-CD47 blocking activity.

### 5.3 Melting Temperatures of Anti-SIRPα Antibodies

The antibodies E1H10B7-CDRV1-IgG1 (YTE), E1H10B7-CDRV1-IgG1, E1F1B1C3-CDRV1-IgG1 (YTE) and E1F1B1C3-CDRV1-IgG1 were also tested for their thermal stability. Briefly, a protein thermal shift assay was used to determine Tm (melting temperature) using a GloMelt™ Thermal Shift Protein Stability Kit (Biotium, Cat #33022-T).

Briefly, the GloMelt™ dye was allowed to thaw and reach room temperature. The vial containing the dye was vortexed and centrifuged. Then, 10×dye was prepared by adding 5 μL 200×dye to 95 μL PBS. Then, 2 μL 10×dye and 10 μg antibodies were added, and PBS was added to a total reaction volume of 20 μL. The tubes containing the dye and antibodies were briefly spun and placed in the real-time PCR thermocycler (Roche, LightCycler 480 II) set up with a melt curve program having the parameters in Table 5.

TABLE 5
Parameters for Melt Curve Program

| Profile step | Temperature | | Ramp rate | Holding Time |
|---|---|---|---|---|
| Initial hold | 25° | C. | NA | 30 s |
| Melt curve | 25-99° | C. | 0.1° C./s | NA |

The results were shown in Table 6, suggesting that the antibodies of the disclosure were probably stable in human body.

TABLE 6
Melting temperatures of anti-SIRPα antibodies

Tm (melting temperature) ° C.

| mAb ID# | Tm1 | Tm2 |
|---|---|---|
| E1H10B7-CDRV1-IgG1 (YTE) | 67.5 | NA |
| E1H10B7-CDRV1-IgG1 | 68.0 | NA |
| E1F1B1C3-CDRV1-IgG1 (YTE) | 78.0 | NA |
| E1F1B1C3-CDRV1-IgG1 | 78.0 | NA |

### Example 6. Cell Based Functional Activity of Anti-SIRPα Antibodies

### 6.1 Anti-SIRPα Antibodies Enhanced Phagocytosis of Raji Cells by Macrophages

CD14+monocytes were isolated from normal human PBMCs by negative selection, and differentiated to macrophages in RPMI-1640 medium supplemented with 10% FBS, 1% penicillin-streptomycin, and 75 ng/mL M-CSF. The macrophages were harvested with Accutase™ on Day 5 and seeded onto 96-well flat bottom plates overnight (10⁴ cells/well, 100 μl). The next day, Raji cells (ATCC #CCL-86, 2×10⁶ cells/mL) were labeled with 1.25 μM CFSE for 20 min at 37° C. and adjusted to the final cell density of 8×10⁵ cells/mL in RPMI-1640 medium supplemented with 10% FBS. The labeled Raji cells (40 μl) and Rituximab (Roche Inc., 4 μg/mL, 40 μl) were added to 96-well U-bottom plates and incubated at room temperature for 30 min. The supernatants in the plates with macrophages were discarded, and 20 μg/mL anti-SIRPα antibodies of the disclosure were added into the plates, 50 μl/well, and incubated at room temperature for 30 min. The mixture of CFSE-labeled Raji cells and Rituximab (50 μl/well) was added into the plates with macrophages and anti-SIRPα antibodies, and incubated at 37° C. for 4 h. And then, all cells were harvested with Accutase™ and transferred into 96-well V-bottom plates. The cells were incubated with Human FcX Blocker (Biolegend, Cat #422302) at 4° C. for 20 min to block Fe receptors, and then incubated with APC-CD11b (Biolegend, Cat #B325845) at 4° C. for 30 min. After centrifugation, the cells were re-suspended in 50 μl staining buffer, and 10 μL PI (Propidium Iodide P3566, Thermo, Cat #2229176) was added. Phagocytosis was measured as the ratio of PI⁻CD11b⁺CFSE⁺ macrophages to the total PI⁻CD11b⁺macrophages. The results were shown in FIG. 27, which showed that the antibodies of the disclosure enhanced phagocytosis of Raji cells by macrophages, with higher activity than BM5.

### 6.2 Anti-SIRPα Antibodies Did Not Induce SIRPα Phosphorylation in THP1 Cells

The anti-SIRPα antibodies of the disclosure were tested in a cell based assay for their agonistic activity. Briefly, 100 μl serially diluted anti-SIRPα antibodies of the disclosure or 100 μl serially diluted human CD47-Fc (in house made with SEQ ID NO: 31) in RPMI-1640 medium supplemented with 10% FBS, 3-fold dilution with the final concentration from 3 nM to 0.004 nM, were plated into 96-well PCR plates. The plates were then added with 10⁶ THP1 cells (ATCC #TIB-202) in 100 μl RPMI-1640 supplemented with 10% FBS, and incubated at 4° C. for 15 min. The plates were centrifuged to remove the supernatants and washed once by PBS. The plates were added with NP-40 lysis buffer with protease/phosphatase inhibitor (1:100), 220 l/well, and incubated on ice for 15 min. The supernatants were collected and stored at −80° C.

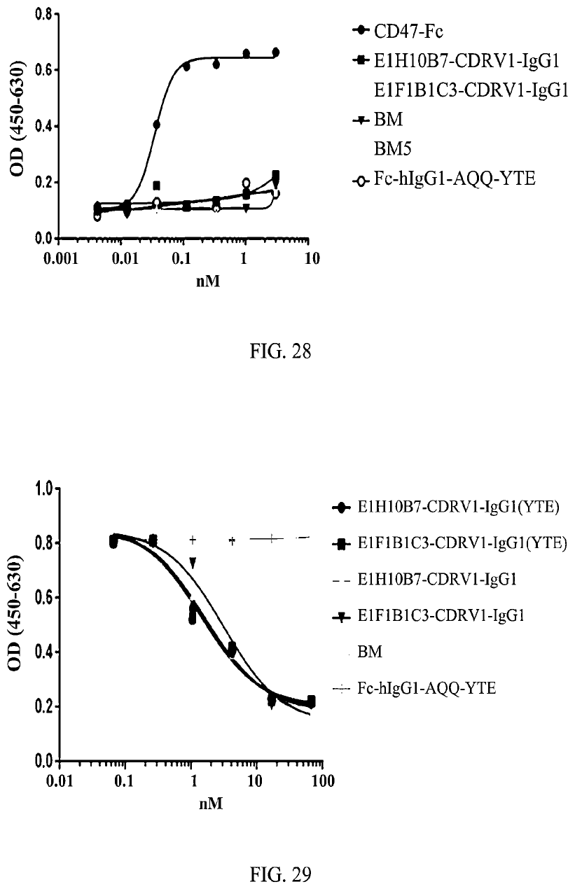

To test the SIRPα protein phosphorylation in cell lysate samples, an anti-SIRPα polyclonal antibody (INVITROGEN, Cat #PA5-81024, 1:1000 in PBS), 100 μl per well, was plated onto 96-well ELISA plates which were incubated at room temperature overnight. The next day, the plates were washed four times with washing buffer (0.05% Tween-20 in PBS, pH7.2 to 7.4) and blocked by 5% milk (250 μl/well) at room temperature for 2 h. The plates were washed four times with washing buffer, added with the cell lysates (100 μl/well) obtained above, and incubated at room temperature for 2 h. After washing four times, the plates were added anti-pY-HRP (RD systems Inc., Cat #HAM1676, 1:5000, 100 μl/well) and incubated at room temperature for 2 h in dark. After washing, the plates were added with TMB (100 μl/well) and incubated at room temperature for about 20 min. OD450 values were measured after addition of 1 M H2SO₄ (50 μl/well). The results were shown in FIG. 28, which showed that the antibodies of the disclosure did not induce SIRPα phosphorylation in THP1 cells, indicating they had no agonistic activity.

### 6.3 Anti-SIRPα Antibodies Antagonized CD47-Mediated Phosphorylation of SIRPα in THP1 Cells

Serially diluted anti-SIRPα antibodies of the disclosure in RPMI-1640 medium supplemented with 10% FBS (4-fold diluted to the final concentration from 66.7 nM to 0.065 nM) were plated onto 96-well plates (50 μl/well). The plates were added with 10⁶ THP1 cells in 100 μl RPMI-1640 supplemented with 10% FBS, incubated at 4° C. for 60 min, added with human CD47-Fc (final concentration at 0.625 nM, 50 μl/well) in RPMI-1640 supplemented with 10% FBS, and incubated at 4° C. for 15 min. The plates were centrifuged to remove the supernatants and the cells in each well were washed once by PBS. Then, NP-40 lysis buffer with protease/phosphatase inhibitor (1:100) was added (220 μl/well), and the plates were incubated on ice for 15 min. The supernatants were collected and stored at −80° C. SIRPα polyclonal antibody (INVITROGEN, Cat #PA5-81024, 1:1000 in PBS), 100 μl per well, was plated onto 96-well ELISA plates at room temperature overnight. The next day, the plates were washed four times with washing buffer (0.05% Tween-20 in PBS, pH7.2 to 7.4) and blocked by 5% milk (250 μl/well) at room temperature for 2 h. The plates were washed four times with washing buffer. And then, the plates were added with the cell lysates obtained above, 100 μl/well, and incubated at room temperature for 2 h. After washing four times, anti-pY-HRP (RD systems Inc., Cat #HAM1676, 1:5000, 100 l/well) was added, and the plates were incubated at room temperature for 2 h in dark. After washing, the plates were added with TMB (100 μl/well) and incubated at room temperature for about 20 min. OD450 values were measured after addition of 1 M H2SO₄ (50 μl/well).

As shown in FIG. 29, the data showed that the antibodies of the disclosure were antagonistic.

### 6.4 Internalization Assay

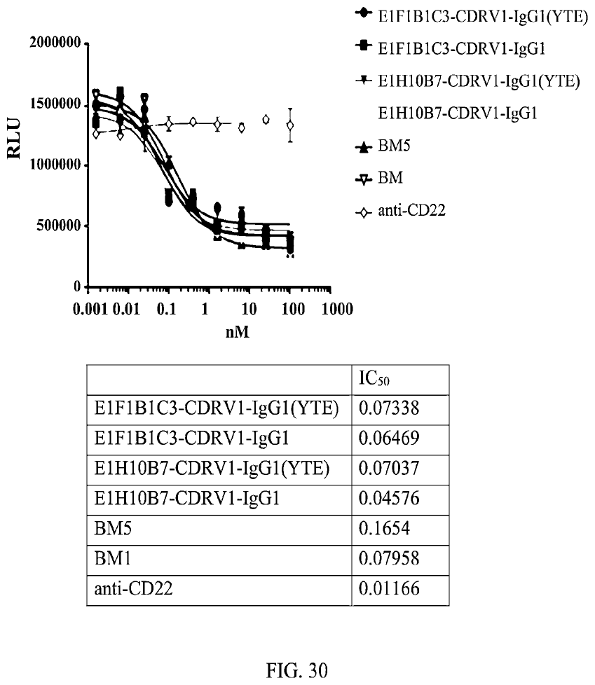

Briefly, 293F-SIRPα cells in 100 μl FreeStyle 293 medium with 10% FBS were plated onto 96-well white/clear bottom plates, 1500 cells/well, and incubated overnight under 5% CO₂ at 37° C. The antibodies of the disclosure and a recombinant toxic protein (DT3C, in house made with SEQ ID NO: 36) were diluted in FreeStyle 293 medium, respectively, mixed (volume ratio 1:1, molar ratio 1:2.2) and incubated at room temperature for 30 min. Serially diluted mixtures were added into the plates with 293F-SIRPα cells, 100 μl per well, and incubated for 3 days under 5% CO₂ at 37° C. Luminescent Cell Viability Assay reagent (Vendor #Vazyme Inc., Cat #DD1101-02) was added, and RLUs were measured (the bottom of the plates was covered with foil). The results were shown in FIG. 30. The results showed that the antibodies of the disclosure were internalized into the cells at similar level and rate to the BM and BM5.

### Example 7. In Vivo Anti-tumor Efficacy of Anti-SIRPα Antibodies in Mouse Tumor Model

7.1 Anti-Tumor Activity of Anti-SIRPα Antibodies in Combination with Anti-CD20 Monoclonal Antibody

The in vivo anti-tumor activity of the antibodies E1H10B7-CDRV1-IgG1 (YTE) and E1F1B1C3-CDRV1-IgG1 (YTE) was tested in B-NDG hSIRPA mice (Beijing Biocytogen Co., Ltd).

Raji cells were purchased from ATCC and genetically engineered by Biocytogen Pharmaceuticals (Beijing) Co., Ltd to express luciferase (termed as B-luc-GFP-Raji cells). The B-luc-GFP-Raji cells were cultured in RPMI 1640 medium containing 10% inactivated fetal bovine serum at 37.0° C. with 5% C02, and injected via the tail vein into 42 female B-NDG hSIRPA mice, 1×10⁵ cells/0.2 mL per mouse. On the 3rd day after Raji cell injection when the average tumor signal intensity reached 1.5×10⁶ p/sec, the mice were randomly assigned into four groups based on the tumor signal intensity and body weight, six per group, and this day was designated as Day 1. From Day 1, the animals in the vehicle group (G2) were injected with Dulbecco's phosphate-buffered saline (also referred to as DPBS), and the animals in group 1 (G1) were intraperitoneally injected with E1H10B7-CDRV1-IgG1 (YTE), twice a week, at the dose of 10 mg/kg, and intravenously injected with Rituximab, an anti-CD20 antibody, every 2 weeks, at 0.1 mg/kg. The animals in group 3 (G3) were intraperitoneally injected with DPBS twice a week and intravenously injected with Rituximab every 2 weeks at 0.1 mg/kg, while the animals in group 4 (G4) were intraperitoneally injected with E1F1B1C3-CDRV1-IgG1 (YTE) twice a week at 10 mg/kg and intravenously injected with Rituximab every 2 weeks at 0.1 mg/kg.

During the study, the tumor signal intensity and the body weight of the mice were measured twice a week. The study was terminated on Day 27. The tumor inhibition rate (TGI) was calculated according to the tumor signal intensity, and the survival curves were generated at the end of the study.

At Day 21, the animals of the vehicle group had a significant body weight loss (−2.2 g, 10.8%) compared to that of Day 1. Further, compared to vehicle group, the body weight changes in the G1 and G3 animals were not significantly different while the average weight of the G4 mice was significantly higher (Table 7-1).

TABLE 7-1
Body weight change of B-NDG hSIRPA mice

| | | | | | Body weight |
|---|---|---|---|---|---|
| Group | | Weight (g)^{a} | | | changes on |
| No. | Drug | Pre-dose | D 21 | P ^{b} | D 21 (g) |
| G1 | E1H10B7-CDRV1-IgG1(YTE) + | 19.7 ± 0.8 | 19.9 ± 0.9 | 0.3355 | +0.2 |
| | Rituximab | | | | |
| G2 | DPBS | 20.3 ± 0.7 | 18.1 ± 0.7 | — | −2.2 |
| G3 | Rituximab | 20.4 ± 0.7 | 19.0 ± 0.8 | 0.8259 | −1.4 |
| G4 | E1F1B1C3-CDRV1-IgG1(YTE) + | 20.2 ± 0.7 | 21.6 ± 0.6 | *0.0140 | +1.4 |
| | Rituximab | | | | |

^{a}Mean ± SEM
^{b} Statistical analysis via One-way ANOVA on the basis of the mouse body weight on Day 21 (administration group versus G2).
*p < 0.05.

TABLE 7-2
Tumor growth inhibition

| Group | | Tumor signal intensity (p/sec)^{a} | | | |
|---|---|---|---|---|---|
| No. | Drug | Before treatment | D 21 | TGI_{TV} (%) | P ^{b} |
| G1 | E1H10B7-CDRV1- | 1.54E+06 ± | 8.39E+08 ± | 76.6 | ***0.0001 |
| | IgG1(YTE) + Rituximab | 1.04E+05 | 1.54E+08 | | |
| G2 | DPBS | 1.56E+06 ± | 3.58E+09 ± | — | — |
| | | 1.74E+05 | 3.08E+08 | | |
| G3 | Rituximab | 1.54E+06 ± | 2.77E+09 ± | 22.5 | 0.2367 |
| | | 1.58E+05 | 5.48E+08 | | |
| G4 | E1F1B1C3-CDRV1- | 1.54E+06 ± | 5.94E+08 ± | 83.4 | ***0.0001 |
| | IgG1(YTE) + Rituximab | 1.70E+05 | 1.46E+08 | | |

^{a}Mean ± SEM
^{b} Statistical analysis via One-way ANOVA on the basis of the tumor signal intensity on Day 21 (administration group versus G2).
***p < 0.001

At Day 21, the average tumor signal intensity of the vehicle group was 3.58×10⁹ p/sec, and those of the G1 and G4 groups were 8.39×10⁸ p/sec and 5.94×10⁸ p/sec, respectively. Further, the TGI_{TV} for G1 and G4 was 76.6% and 83.4%, respectively (Table 7-2).

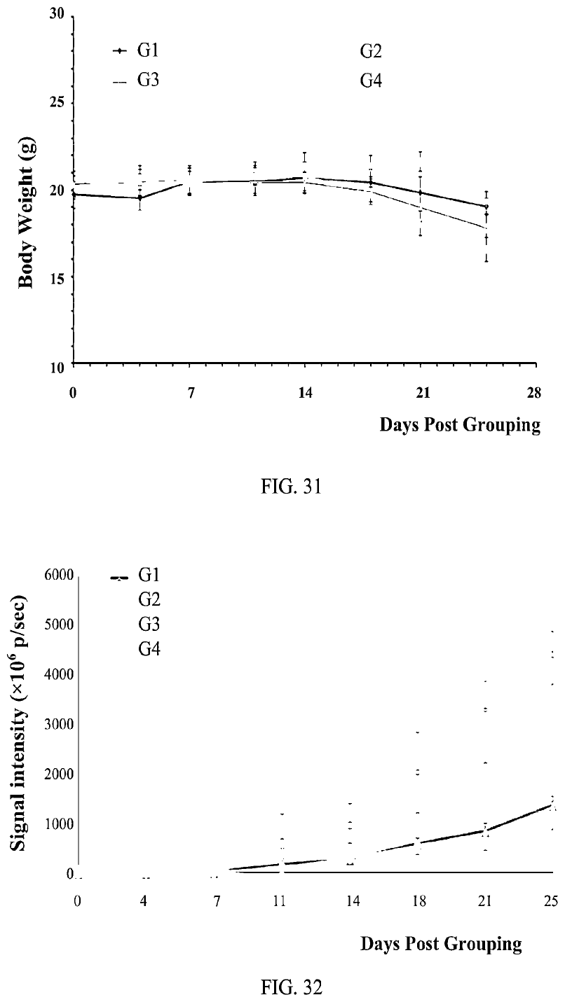

As can be seen from Tables 7-1, 7-2 and FIGS. 31-32, the anti-SIRPα antibodies of the disclosure at the dose of 10 mg/kg showed high efficacy when used in combination with Rituximab.

### 7.2 Dose Dependent Anti-Tumor Activity of Anti-SIRPα Antibodies in Animal Model

The in vivo anti-tumor activity of E1F1B1C3-CDRV1-IgG1 (YTE) was further tested according to the protocol as described in 7.1.

TABLE 7-3
Tumor growth inhibition

| Group | | Tumor signal intensity (p/sec)^{a} | | | |
|---|---|---|---|---|---|
| No. | Drug | Before treatment | D 17 | TGI_{TV} (%) | P ^{b} |
| G1 | Rituximab | 1.42E+06 ± | 2.28E+09 ± | 31.31 | 0.1538 |
| | | 1.93E+05 | 4.96E+08 | | |
| G2 | DPBS | 1.43E+06 ± | 3.32E+09 ± | / | / |
| | | 1.40E+05 | 4.56E+08 | | |
| G3 | E1F1B1C3-CDRV1-IgG1 | 1.42E+06 ± | 9.12E+07 ± | 97.25 | 0.00003*** |
| | (YTE) + Rituximab | 2.04E+05 | 3.72E+07 | | |
| G4 | BM5 + Rituximab | 1.41E+06 ± | 2.93E+08 ± | 91.18 | 0.00008*** |
| | | 1.59E+05 | 1.42E+08 | | |
| G5 | E1F1B1C3-CDRV1-IgG1 | 1.42E+06 ± | 6.23E+08 ± | 81.25 | 0.0007*** |
| | (YTE) + Rituximab | 1.90E+05 | 3.28E+08 | | |

^{a}Mean ± SEM
^{b} Statistical analysis via One-way ANOVA on the basis of the tumor signal intensity on Day 17 (administration group versus G2).
***p < 0.001

Specifically, the mice were randomly assigned into five groups (six for each group) based on the tumor signal intensity and body weight. From Day 1, the animals in the vehicle group (G2) were injected with DPBS; the animals in group 3 (G3) were intraperitoneally injected with E1F1B1C3-CDRV1-IgG1 (YTE), twice a week, at the dose of 10 mg/kg, and intravenously injected with Rituximab, every 2 weeks, at 0.1 mg/kg; and the animals in group 5 (G5) were intraperitoneally injected with E1F1B1C3-CDRV1-IgG1 (YTE), twice a week, at the dose of 3 mg/kg, and intravenously injected with Rituximab, every 2 weeks, at 0.1 mg/kg. The animals in group 1 (G1) were intraperitoneally injected with DPBS twice a week and intravenously injected with Rituximab every 2 weeks at 0.1 mg/kg, while the animals in group 4 (G4) were intraperitoneally injected with BM5 twice a week at 10 mg/kg and intravenously injected with Rituximab every 2 weeks at 0.1 mg/kg. The results are showed in Table 7-3.

At Day 17, the average tumor signal intensity of the vehicle group was 3.32×10⁹ p/sec, and those of the G3 and G5 groups were 9.12×10⁷ p/sec and 6.23×10⁸ p/sec, respectively. And the TGI_{TV} for G3 and G5 was 97.25% and 81.25%, respectively (Table 7-3).

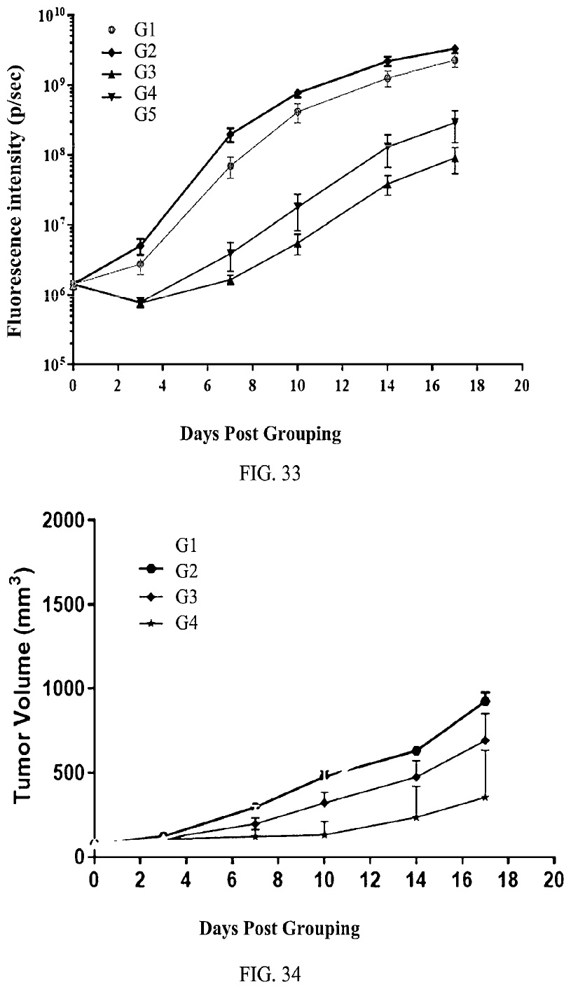

As can be seen from Table 7-3 and FIG. 33, the anti-SIRPα antibodies of the disclosure showed anti-tumor activity in a dose dependent manner. Moreover, the anti-tumor efficacy of the anti-SIRPα antibodies of the disclosure was higher than that of BM5 at the dose of 10 mg/kg when used in combination with Rituximab.

### 7.3 Anti-Tumor Activity of Anti-SIRPα Antibodies in Solid Tumor Model

The in vivo anti-tumor activity of E1F1B1C3-CDRV1-IgG1 (YTE) was tested in C57BL/6-hSIRPA(2)/hCD47 mice (Shanghai Model Organisms Center, Inc.) implanted with colon tumors.

MC38-hCLDN18.2/hCD47 cells, a murine colon cancer cell line (NM-S13-TM19, Shanghai Model Organisms Center, Inc.), were cultured in EMEM/High+10% fetal bovine serum medium at 37° C. with 5% CO₂, and injected subcutaneously via the right flank into 48 female C57BL/6-hSIRPA(2)/hCD47 mice, 3×10⁶ cells/0.1 mL per mouse. When the average tumor volume reached 75-95 mm³, the mice were randomly assigned into four groups based on the tumor volume and body weight, eight per group, and this day was designated as Day 1.

From Day 1, the animals in the vehicle group (G2) were injected with DPBS intraperitoneally, and the animals in group 1 (G1) were intraperitoneally injected with an anti-CLDN18.2 antibody (in house made with the heavy and light chain of SEQ ID NOs: 39 and 40), twice a week, at the dose of 10 mg/kg. The animals in group 3 (G3) were intraperitoneally injected with E1F1B1C3-CDRV1-IgG1 (YTE) twice a week at 10 mg/kg, while the animals in group 4 (G4) were intraperitoneally injected with E1F1B1C3-CDRV1-IgG1 (YTE) twice a week at 10 mg/kg and anti-CLDN18.2 antibody twice a week at 10 mg/kg.

During the study, the tumor volume and the body weight were measured twice a week. The tumor inhibition rate (TGI) was calculated according to the tumor volume, and the results are showed in table 7-4.

At Day 17, the average tumor volume of the vehicle group was 924.72 mm³, and those of the G3 and G4 groups were 693.94 mm³ and 355.15 mm³, respectively. The TGI_{TV} for G3 and G4 was 23.83% and 66.05%, respectively.

As can be seen from Tables 7-4 and FIG. 34, the anti-SIRPα antibodies of the disclosure showed high anti-tumor activity when combined with the anti-CLDN18.2 antibody in suppressing solid tumor growth.

TABLE 7-4
Tumor growth inhibition

| Group | | Tumor Volume (mm³)^{a} | | | |
|---|---|---|---|---|---|
| No. | Drug | Before treatment | D 17 | TGI_{TV} (%) | P ^{b} |
| G1 | anti-CLDN18.2 | 79.40 ± 2.99 | 1112.58 ± 225.10 | −16.95 | 0.4685 |
| G2 | DPBS | 80.15 ± 3.54 | 924.72 ± 49.10 | / | / |
| G3 | E1F1B1C3-CDRV1-IgG1 | 79.45 ± 2.93 | 693.94 ± 147.58 | 23.83 | 0.2003 |
| | (YTE) | | | | |
| G4 | E1F1B1C3-CDRV1-IgG1 | 79.50 ± 2.86 | 355.15 ± 262.39 | 66.05 | 0.0095** |
| | (YTE) + anti-CLDN18.2 | | | | |

^{a}Mean ± SEM
^{b} Statistical analysis via One-way ANOVA on the basis of the tumor size on Day 17 (administration group versus G2).
**p < 0.01

While the disclosure has been described above in connection with one or more embodiments, it should be understood that the disclosure is not limited to those embodiments, and the description is intended to cover all alternatives, modifications, and equivalents, as may be included within the spirit and scope of the appended claims. All referenced cited herein are further incorporated by reference in their entirety.

Sequences in the present application are summarized below.

Description/
Sequence/SEQ ID NO.
VH CDR1 of chimeric and fully human E1F1B1C3, E1F1B1C3-CDRV1, and E1F1B1C3-CDRV2
SNAMH (SEQ ID NO: 1)
VH CDR2 of chimeric and fully human E1F1B1C3
WIKAGX1X2EIKYSQKFQG (SEQ ID NO: 2) X1 = N, X2 = G
WIKAGNGEIKYSQKFQG
VH CDR2 of E1F1B1C3-CDRV1
SEQ ID NO: 2, X1 = N, X2 = A
WIKAGNAEIKYSQKFQG
VH CDR2 of E1F1B1C3-CDRV2
SEQ ID NO: 2, X1 = A, X2 = G
WIKAGAGEIKYSQKFQG
VH CDR3 of chimeric and fully human E1F1B1C3, E1F1B1C3-CDRV1, and E1F1B1C3-CDRV2
GDYYYYYMDV (SEQ ID NO: 3)
VL CDR1 for chimeric and fully human E1F1B1C3, E1F1B1C3-CDRV1, and E1F1B1C3-CDRV2
RASQSISDFLN (SEQ ID NO: 4)
VL CDR2 of chimeric and fully human E1F1B1C3
AASSLQX (SEQ ID NO: 5) X = N
AASSLQN
VH CDR2 of E1F1B1C3-CDRV1 and E1F1B1C3-CDRV2
SEQ ID NO: 5, X = A
AASSLQA
VL CDR3 of chimeric and fully human E1F1B1C3, E1F1B1C3-CDRV1, and E1F1B1C3-CDRV2
QQSYSTPLS (SEQ ID NO: 6)
VH of chimeric and human E1F1B1C3
QVQLVQSGAEVKKPGASVKVSCKASGYTFTSNAMHWVRQVPGQGLEWMGWIKAGX1X2EIK
YSQKFQGRVTITRDTSATTAYMELSSLRSEDTAVYYCARGDYYYYYMDVWGKGTTVTVSS
(SEQ ID NO: 7) X1 = N, X2 = G
QVQLVQSGAEVKKPGASVKVSCKASGYTFTSNAMHWVRQVPGQGLEWMGWIKAGNGEIKYS
QKFQGRVTITRDTSATTAYMELSSLRSEDTAVYYCARGDYYYYYMDVWGKGTTVTVSS
VH of E1F1B1C3-CDRV1
SEQ ID NO: 7, X1 = N, X2 = A
QVQLVQSGAEVKKPGASVKVSCKASGYTFTSNAMHWVRQVPGQGLEWMGWIKAGNAEIKYS
QKFQGRVTITRDTSATTAYMELSSLRSEDTAVYYCARGDYYYYYMDVWGKGTTVTVSS
VH of E1F1B1C3-CDRV2
SEQ ID NO: 7, X1 = A, X2 = G
QVQLVQSGAEVKKPGASVKVSCKASGYTFTSNAMHWVRQVPGQGLEWMGWIKAGAGEIKYS
QKFQGRVTITRDTSATTAYMELSSLRSEDTAVYYCARGDYYYYYMDVWGKGTTVTVSS
VL of chimeric and human E1F1B1C3
DIQVTQSPSSLSASVGDRVTITCRASQSISDFLNWYQQKPGKAPKLLIHAASSLQXGVPSRFSGS
GSGTDFSLTINSLQPEDFATYYCQQSYSTPLSFGGGTKVEIK (SEQ ID NO: 8) X = N
DIQVTQSPSSLSASVGDRVTITCRASQSISDFLNWYQQKPGKAPKLLIHAASSLQNGVPSRFSGS
GSGTDFSLTINSLQPEDFATYYCQQSYSTPLSFGGGTKVEIK
VL of E1F1B1C3-CDRV1 and E1F1B1C3-CDRV2
SEQ ID NO: 8, X = A
DIQVTQSPSSLSASVGDRVTITCRASQSISDFLNWYQQKPGKAPKLLIHAASSLQAGVPSRFSGS
GSGTDFSLTINSLQPEDFATYYCQQSYSTPLSFGGGTKVEIK
VH CDR1 of chimeric and fully human E1H10B7C7, E1H10B7-CDRV1, and E1H10B7-CDRV2
SXAMQ (SEQ ID NO: 9) X = Y
SYAMQ
VH CDR2 of chimeric and fully human E1H10B7C7
WIIAGX1X2DTKYSQNFQD (SEQ ID NO: 10) X1 = N, X2 = G
WIIAGNGDTKYSQNFQD
VH CDR2 of E1H10B7-CDRV1
SEQ ID NO: 10, X1 = N, X2 = A
WIIAGNADTKYSQNFQD
VH CDR2 of E1H10B7-CDRV2
SEQ ID NO: 10, X1 = A, X2 = G
WIIAGAGDTKYSQNFQD
VH CDR3 of chimeric and fully human E1H10B7C7, E1H10B7-CDRV1, and E1H10B7-CDRV2
GSGFSGYGYFDK (SEQ ID NO: 11)
VL CDR1 of chimeric and fully human E1H10B7C7, E1H10B7-CDRV1, and E1H10B7-CDRV2
RASHSISSYLN (SEQ ID NO: 12)
VL CDR2 of chimeric and fully human E1H10B7C7, E1H10B7-CDRV1, and E1H10B7-CDRV2
ASSSLQS (SEQ ID NO: 13)
VL CDR3 of chimeric and fully human E1H10B7C7, E1H10B7-CDRV1, and E1H10B7-CDRV2
QQSHSIPLT (SEQ ID NO: 14)
VH of chimeric and fully human E1H10B7C7
QVQLVQSGAEVKKPGASVKVSCKASGYTFTSYAMQWVRQAPGQRLEWMGWIIAGX1X2DTK
YSQNFQDRVTLTRDTSANTAYMDLSGLRSEDTAVYYCARGSGFSGYGYFDKWGQGTLVTVSS
(SEQ ID NO: 15) X1 = N, X2 = G
QVQLVQSGAEVKKPGASVKVSCKASGYTFTSYAMQWVRQAPGQRLEWMGWIIAGNGDTKYS
QNFQDRVTLTRDTSANTAYMDLSGLRSEDTAVYYCARGSGFSGYGYFDKWGQGTLVTVSS
VH of E1H10B7-CDRV1
SEQ ID NO: 15, X1 = N, X2 = A
QVQLVQSGAEVKKPGASVKVSCKASGYTFTSYAMQWVRQAPGQRLEWMGWIIAGNADTKYS
QNFQDRVTLTRDTSANTAYMDLSGLRSEDTAVYYCARGSGFSGYGYFDKWGQGTLVTVSS
VH of E1H10B7-CDRV2
SEQ ID NO: 15, X1 = A, X2-G
QVQLVQSGAEVKKPGASVKVSCKASGYTFTSYAMQWVRQAPGQRLEWMGWIIAGAGDTKYS
QNFQDRVTLTRDTSANTAYMDLSGLRSEDTAVYYCARGSGFSGYGYFDKWGQGTLVTVSS
VL of chimeric and fully human E1H10B7C7
DIQMTQSPSSLSASVGDRVTITCRASHSISSYLNWYQQKPGKVPKLLIYASSSLQSGVPSRFSGX
GSGTDFTLIISSLQPEDFATYYCQQSHSIPLTFGGGTKVEIK (SEQ ID NO: 16) X = N
DIQMTQSPSSLSASVGDRVTITCRASHSISSYLNWYQQKPGKVPKLLIYASSSLQSGVPSRFSGN
GSGTDFTLIISSLQPEDFATYYCQQSHSIPLTFGGGTKVEIK
VL of E1H10B7-CDRV1 and E1H10B7-CDRV2
SEQ ID NO: 16, X = S
DIQMTQSPSSLSASVGDRVTITCRASHSISSYLNWYQQKPGKVPKLLIYASSSLQSGVPSRFSGSG
SGTDFTLIISSLQPEDFATYYCQQSHSIPLTFGGGTKVEIK
VH CDR1 of chimeric and human E1D5E1F1
SEQ ID NO: 9, X = F
SFAMQ
VH CDR2 of chimeric and human E1D5E1F1
SEQ ID NO: 10, X1 = N, X2 = G
WIIAGNGDTKYSQNFQD
VH CDR3 of chimeric and human E1D5E1F1
GSGFSGYGYFDK (SEQ ID NO: 11)
VL CDR1 of chimeric and human E1D5E1F1
RASHSISSYLN (SEQ ID NO: 12)
VL CDR2 of chimeric and human E1D5E1F1
ASSSLQS (SEQ ID NO: 13)
VL CDR3 of chimeric and human E1D5E1F1
QQSHSIPLT (SEQ ID NO: 14)
VH of chimeric and human E1D5E1F1
QVQLVQSGAEVKKPGASVKVSCKASGYTFTSFAMQWVRRAPGQRLEWMGWIIAGNGDTKYS
QNFQDRVTLTRDTSANTAYMDLSGLRSEDTAVYYCARGSGFSGYGYFDKWGQGTLVTVSS
(SEQ ID NO: 17)
VL of chimeric and human E1D5E1F1
DIQMTQSPSSLSASVGDRVTITCRASHSISSYLNWYQQKPGKVPKLLIYASSSLQSGVPSRFSGN
GSGTDFTLIISSLQPEDFATYYCQQSHSIPLTFGGGTKVEIK (SEQ ID NO: 18)
Heavy chain constant region-IgG4 (S228P + YTE + K447 Del)
ASTKGPSVFPLAPCSRSTSESTAALGCLVKDYFPEPVTVSWNSGALTSGVHTFPAVLQSSGLYSL
SSVVTVPSSSLGTKTYTCNVDHKPSNTKVDKRVESKYGPPCPPCPAPEFLGGPSVFLFPPKPKDT
LYITREPEVTCVVVDVSQEDPEVQFNWYVDGVEVHNAKTKPREEQFNSTYRVVSVLTVLHQD
WLNGKEYKCKVSNKGLPSSIEKTISKAKGQPREPQVYTLPPSQEEMTKNQVSLTCLVKGFYPSD
IAVEWESNGQPENNYKTTPPVLDSDGSFFLYSRLTVDKSRWQEGNVFSCSVMHEALHNHYTQ
KSLSLSLG (SEQ ID NO: 19)
Light chain constant region
RTVAAPSVFIFPPSDEQLKSGTASVVCLLNNFYPREAKVQWKVDNALQSGNSQESVTEQDSKD
STYSLSSTLTLSKADYEKHKVYACEVTHQGLSSPVTKSFNRGEC (SEQ ID NO: 20)
Human SIRPα-Fc protein
MEPAGPAPGRLGPLLCLLLAASCAWSGVAGEEELQVIQPDKSVLVAAGETATLRCTATSLIPVG
PIQWFRGAGPGRELIYNQKEGHFPRVTTVSDLTKRNNMDFSIRIGNITPADAGTYYCVKFRKGS
PDDVEFKSGAGTELSVRAKPSAPVVSGPAARATPQHTVSFTCESHGFSPRDITLKWFKNGNELS
DFQTNVDPVGESVSYSIHSTAKVVLTREDVHSQVICEVAHVTLQGDPLRGTANLSETIRVPPTLE
VTQQPVRAENQVNVTCQVRKFYPQRLQLTWLENGNVSRTETASTVTENKDGTYNWMSWLLV
NVSAHRDDVKLTCQVEHDGQPAVSKSHDLKVSAHPKEQGSNTAAENTGSNEREPKSCDKTHT
CPPCPAPELLGGPSVFLFPPKPKDTLMISRTPEVTCVVVDVSHEDPEVKFNWYVDGVEVHNAKT
KPREEQYNSTYRVVSVLTVLHQDWLNGKEYKCKVSNKALPAPIEKTISKAKGQPREPQVYTLP
PSREEMTKNQVSLTCLVKGFYPSDIAVEWESNGQPENNYKTTPPVLDSDGSFFLYSKLTVDKSR
WQQGNVFSCSVMHEALHNHYTQKSLSLSPGK (SEQ ID NO: 21)
Human SIRPα-his
MEPAGPAPGRLGPLLCLLLAASCAWSGVAGEEELQVIQPDKSVLVAAGETATLRCTATSLIPVG
PIQWFRGAGPGRELIYNQKEGHFPRVTTVSDLTKRNNMDFSIRIGNITPADAGTYYCVKFRKGS
PDDVEFKSGAGTELSVRAKPSAPVVSGPAARATPQHTVSFTCESHGFSPRDITLKWFKNGNELS
DFQTNVDPVGESVSYSIHSTAKVVLTREDVHSQVICEVAHVTLQGDPLRGTANLSETIRVPPTLE
VTQQPVRAENQVNVTCQVRKFYPQRLQLTWLENGNVSRTETASTVTENKDGTYNWMSWLLV
NVSAHRDDVKLTCQVEHDGQPAVSKSHDLKVSAHPKEQGSNTAAENTGSNERHHHHHHHHH
H (SEQ ID NO: 22)
Human SIRPβ-his
MDMRVPAQLLGLLLLWFPGSRCEDELQVIQPEKSVSVAAGESATLRCAMTSLIPVGPIMWFRG
AGAGRELIYNQKEGHFPRVTTVSELTKRNNLDFSISISNITPADAGTYYCVKFRKGSPDDVEFKS
GAGTELSVRAKPSAPVVSGPAVRATPEHTVSFTCESHGFSPRDITLKWFKNGNELSDFQTNVDP
AGDSVSYSIHSTARVVLTRGDVHSQVICEIAHITLQGDPLRGTANLSEAIRVPPTLEVTQQPMRA
ENQANVTCQVSNFYPRGLQLTWLENGNVSRTETASTLIENKDGTYNWMSWLLVNTCAHRDD
VVLTCQVEHDGQQAVSKSYALEISAHQKEHGSDITHEAALAPTAPLHHHHHHHHHH (SEQ ID
NO: 23)
Human SIRPγ-his
MPVPASWPHPPGPFLLLTLLLGLTEVAGEEELQMIQPEKLLLVTVGKTATLHCTVTSLLPVGPV
LWFRGVGPGRELIYNQKEGHFPRVTTVSDLTKRNNMDFSIRISSITPADVGTYYCVKFRKGSPE
NVEFKSGPGTEMALGAKPSAPVVLGPAARTTPEHTVSFTCESHGFSPRDITLKWFKNGNELSDF
QTNVDPTGQSVAYSIRSTARVVLDPWDVRSQVICEVAHVTLQGDPLRGTANLSEAIRVPPTLEV
TQQPMRVGNQVNVTCQVRKFYPQSLQLTWSENGNVCQRETASTLTENKDGTYNWTSWFLVN
ISDQRDDVVLTCQVKHDGQLAVSKRLALEVTVHQKDQSSDATPGPASHHHHHH (SEQ ID NO:
24)
Cyno SIRPα-his
MEPAGPAPGRLGPLLCLLLTASCAWSGVLGEEELQVIQPEKSVSVAAGDSATLNCTVSSLIPVG
PIQWFRGAGPGRELIYNLKEGHFPRVTAVSDPTKRNNMDFSIRISNITPADAGTYYCVKFRKGSP
DVELKSGAGTELSVRAKPSAPVVSGPAVRATAEHTVSFTCESHGFSPRDITLKWFKNGNELSDV
QTNVDPAGKSVSYSIRSTARVLLTRRDVHSQVICEVAHVTLQGDPLRGTANLSEAIRVPPFLEVT
QQSMRADNQVNVTCQVTKFYPQRLQLTWLENGNVSRTEMASALPENKDGTYNWTSWLLVN
VSAHRDDVKLTCQVEHDGQPAVNKSFSVKVSAHPKEQGSNTAAENTGTNERHHHHHHHHHH
(SEQ ID NO: 25)
Full length human SIRPα
MEPAGPAPGRLGPLLCLLLAASCAWSGVAGEEELQVIQPDKSVLVAAGETATLRCTATSLIPVG
PIQWFRGAGPGRELIYNQKEGHFPRVTTVSDLTKRNNMDFSIRIGNITPADAGTYYCVKFRKGS
PDDVEFKSGAGTELSVRAKPSAPVVSGPAARATPQHTVSFTCESHGFSPRDITLKWFKNGNELS
DFQTNVDPVGESVSYSIHSTAKVVLTREDVHSQVICEVAHVTLQGDPLRGTANLSETIRVPPTLE
VTQQPVRAENQVNVTCQVRKFYPQRLQLTWLENGNVSRTETASTVTENKDGTYNWMSWLLV
NVSAHRDDVKLTCQVEHDGQPAVSKSHDLKVSAHPKEQGSNTAAENTGSNERNIYIVVGVVC
TLLVALLMAALYLVRIRQKKAQGSTSSTRLHEPEKNAREITQDTNDITYADLNLPKGKKPAPQA
AEPNNHTEYASIQTSPQPASEDTLTYADLDMVHLNRTPKQPAPKPEPSFSEYASVQVPRKYPYD
VPDYA (SEQ ID NO: 26)
Full length human CD47
MWPLVAALLLGSACCGSAQLLFNKTKSVEFTFCNDTVVIPCFVTNMEAQNTTEVYVKWKFKG
RDIYTFDGALNKSTVPTDFSSAKIEVSQLLKGDASLKMDKSDAVSHTGNYTCEVTELTREGETII
ELKYRVVSWFSPNENILIVIFPIFAILLFWGQFGIKTLKYRSGGMDEKTIALLVAGLVITVIVIVGA
ILFVPGEYSLKNATGLGLIVTSTGILILLHYYVFSTAIGLTSFVIAILVIQVIAYILAVVGLSLCIAA
CIPMHGPLLISGLSILALAQLLGLVYMKFVASNQKTIQPPRNN (SEQ ID NO: 27)
Human SIRPα-isoform2-his
MGWSCIILFLVATATGVHSEEELQVIQPDKSVSVAAGESAILHCTVTSLIPVGPIQWFRGAGPAR
ELIYNQKEGHFPRVTTVSESTKRENMDFSISISNITPADAGTYYCVKFRKGSPDTEFKSGAGTELS
VRAKPSAPVVSGPAARATPQHTVSFTCESHGFSPRDITLKWFKNGNELSDFQTNVDPVGESVSY
SIHSTAKVVLTREDVHSQVICEVAHVTLQGDPLRGTANLSETIRVPPTLEVTQQPVRAENQVNV
TCQVRKFYPQRLQLTWLENGNVSRTETASTVTENKDGTYNWMSWLLVNVSAHRDDVKLTCQ
VEHDGQPAVSKSHDLKVSAHPKEQGSNTAAENTGSNERHHHHHHHHHH (SEQ ID NO: 28)
Human SIRPα-V8-his
MGWSCIILFLVATATGVHSGVAGEEELQVIQPDKSVLVAAGETATLRCTATSLIPVGPIQWFRG
AGPARELIYNQKEGHFPRVTTVSESTKRENMDFSISISNITPADAGTYYCVKFRKGSPDTEFKSG
AGTELSVRAKPSAPVVSGPAARATPQHTVSFTCESHGFSPRDITLKWFKNGNELSDFQTNVDPV
GESVSYSIHSTAKVVLTREDVHSQVICEVAHVTLQGDPLRGTANLSETIRVPPTLEVTQQPVRAE
NQVNVTCQVRKFYPQRLQLTWLENGNVSRTETASTVTENKDGTYNWMSWLLVNVSAHRDD
VKLTCQVEHDGQPAVSKSHDLKVSAHPKEQGSNTAAENTGSNERHHHHHHHHHH (SEQ ID
NO: 29)
Human CD47-his
MDMRVPAQLLGLLLLWFPGSRCQLLFNKTKSVEFTFCNDTVVIPCFVTNMEAQNTTEVYVKW
KFKGRDIYTFDGALNKSTVPTDFSSAKIEVSQLLKGDASLKMDKSDAVSHTGNYTCEVTELTRE
GETIIELKYRVVSWFSPHHHHHHHHHH (SEQ ID NO: 30)
Human CD47-Fc
QLLFNKTKSVEFTFCNDTVVIPCFVTNMEAQNTTEVYVKWKFKGRDIYTFDGALNKSTVPTDF
SSAKIEVSQLLKGDASLKMDKSDAVSHTGNYTCEVTELTREGETIIELKYRVVSWFSPEPKSCD
KTHTCPPCPAPELLGGPSVFLFPPKPKDTLMISRTPEVTCVVVDVSHEDPEVKFNWYVDGVEVH
NAKTKPREEQYNSTYRVVSVLTVLHQDWLNGKEYKCKVSNKALPAPIEKTISKAKGQPREPQV
YTLPPSREEMTKNQVSLTCLVKGFYPSDIAVEWESNGQPENNYKTTPPVLDSDGSFFLYSKLTV
DKSRWQQGNVFSCSVMHEALHNHYTQKSLSLSPGK* (SEQ ID NO: 31)
Heavy chain of BM
EVQLQQSGAELVKPGASVKLSCTASGFNIKDYYIHWVQQRTEQGLEWIGRIDPEDGETKYAPK
FQDKATITADTSSNTAYLHLSSLTSEDTAVYYCARWGAYWGQGTLVTVSAASTKGPSVFPLAP
CSRSTSESTAALGCLVKDYFPEPVTVSWNSGALTSGVHTFPAVLQSSGLYSLSSVVTVPSSSLGT
KTYTCNVDHKPSNTKVDKRVESKYGPPCPPCPAPEFLGGPSVFLFPPKPKDTLMISRTPEVTCVV
VDVSQEDPEVQFNWYVDGVEVHNAKTKPREEQFNSTYRVVSVLTVLHQDWLNGKEYKCKVS
NKGLPSSIEKTISKAKGQPREPQVYTLPPSQEEMTKNQVSLTCLVKGFYPSDIAVEWESNGQPEN
NYKTTPPVLDSDGSFFLYSRLTVDKSRWQEGNVFSCSVMHEALHNHYTQKSLSLSLGK (SEQ
ID NO: 32)
Light chain of BM
QIVLTQSPAIMSASPGEKVTLTCSASSSVSSSYLYWYQQKPGSSPKLWIYSTSNLASGVPARFSG
SGSGTSYSLTISSMEAEDAASYFCHQWSSYPRTFGAGTKLELKRTVAAPSVFIFPPSDEQLKSGT
ASVVCLLNNFYPREAKVQWKVDNALQSGNSQESVTEQDSKDSTYSLSSTLTLSKADYEKHKV
YACEVTHQGLSSPVTKSFNRGEC (SEQ ID NO: 33)
Heavy chain of BM5
EVQLVQSGAEVKKPGESLRISCKASGYSFTSYWVHWVRQMPGKGLEWMGNIDPSDSDTHYSP
SFQGHVTLSVDKSISTAYLQLSSLKASDTAMYYCVRGGTGTLAYFAYWGQGTLVTVSSASTKG
PSVFPLAPCSRSTSESTAALGCLVKDYFPEPVTVSWNSGALTSGVHTFPAVLQSSGLYSLSSVVT
VPSSSLGTKTYTCNVDHKPSNTKVDKRVESKYGPPCPPCPAPEFLGGPSVFLFPPKPKDTLMISR
TPEVTCVVVDVSQEDPEVQFNWYVDGVEVHNAKTKPREEQFNSTYRVVSVLTVLHQDWLNG
KEYKCKVSNKGLPSSIEKTISKAKGQPREPQVYTLPPSQEEMTKNQVSLTCLVKGFYPSDIAVE
WESNGQPENNYKTTPPVLDSDGSFFLYSRLTVDKSRWQEGNVFSCSVMHEALHNHYTQKSLSL
SPGK (SEQ ID NO: 34)
Light chain of BM5
DVVMTQSPLSLPVTLGQPASISCRSSQSLVHSYGNTYLYWFQQRPGQSPRLLIYRVSNRFSGVP
DRFSGSGSGTDFTLKISRVEAEDVGVYYCFQGTHVPYTFGGGTKVEIKRTVAAPSVFIFPPSDEQ
LKSGTASVVCLLNNFYPREAKVQWKVDNALQSGNSQESVTEQDSKDSTYSLSSTLTLSKADYE
KHKVYACEVTHQGLSSPVTKSFNRGEC (SEQ ID NO: 35)
DT3C protein
MGADDVVDSSKSFVMENFSSYHGTKPGYVDSIQKGIQKPKSGTQGNYDDDWKGFYSTDNKY
DAAGYSVDNENPLSGKAGGVVKVTYPGLTKVLALKVDNAETIKKELGLSLTEPLMEQVGTEEF
IKRFGDGASRVVLSLPFAEGSSSVEYINNWEQAKALSVELEINFETRGKRGQDAMYEYMAQAC
AGNRVRRSVGSSLSCINLDWDVIRDKTKTKIESLKEHGPIKNKMSESPNKTVSEEKAKQYLEEF
HQTALEHPELSELKTVTGTNPVFAGANYAAWAVNVAQVIDSETADNLEKTTAALSILPGIGSV
MGIADGAVHHNTEEIVAQSIALSSLMVAQAIPLVGELVDIGFAAYNFVESIINLFQVVHNSYNRP
AYSPGHKHIDEILAALPKTDTYKLILNGKTLKGETTTEAVDAATAEKVFKQYANDNGVDGEWT
YDDATKTFTVTEKPEVIDASELTPAVTTYKLVINGKTLKGETTTEAVDAATAEKVFKQYANDN
GVDGEWTYDDATKTFTVTEKPEVIDASELTPAVTTYKLVINGKTLKGETTTKAVDAETAEKAF
KQYANDNGVDGVWTYDDATKTFTVTELEHHHHHH (SEQ ID NO: 36)
mouse SIRPα-his
MEPAGPAPGRLGPLLLCLLLSASCFCTGATGTEVKVTQPEKSVSVAAGDSTILNCTVTSLLPVGP
IRWYRGVGQSRLLIYSFTGEHFPRVRNVSDTTKRNNMDFSIRISNVTPEDAGTYYCVKFQRGSS
EPDTEIQSGGGTEVYVLAKPSPPEVSGPADRGIPDQKVNFTCKSHGFSPRNITLKWFKDGQELHP
LETTVNPSGKNVSYNISSTVRVVLNSMDVNSKVICEVAHITLDRSPLRGIANLSNFIRVSPTVKV
TQQPPTSMNQVNLTCRAERFYPEDLQLIWLENGNVSRNDTPKNLTKNTDGTYNYTSLFLVNSS
AHREDVVFTCQVKHDQQPAITRNHTVLGLAHSSDQGSMQTFHHHHHHHHHH* (SEQ ID NO: 37)
Heavy chain constant region-IgG1(AQQ + K447 Del)
ASTKGPSVFPLAPSSKSTSGGTAALGCLVKDYFPEPVTVSWNSGALTSGVHTFPAVLQSSGLYS
LSSVVTVPSSSLGTQTYICNVNHKPSNTKVDKKVEPKSCDKTHTCPPCPAPEAQGGPSVFLFPPK
PKDTLX1IX2RX3PEVTCVVVDVSHEDPEVKFNWYVDGVEVHNAKTKPREEQYNSTYRVVSVL
TVLHQDWLNGKEYKCQVSNKALPAPIEKTISKAKGQPREPQVYTLPPSRDELTKNQVSLTCLV
KGFYPSDIAVEWESNGQPENNYKTTPPVLDSDGSFFLYSKLTVDKSRWQQGNVFSCSVMHEAL
HNHYTQKSLSLSPG (SEQ ID NO: 38) X1 = M, X2 = S, X3 = T
ASTKGPSVFPLAPSSKSTSGGTAALGCLVKDYFPEPVTVSWNSGALTSGVHTFPAVLQSSGLYS
LSSVVTVPSSSLGTQTYICNVNHKPSNTKVDKKVEPKSCDKTHTCPPCPAPEAQGGPSVFLFPPK
PKDTLMISRTPEVTCVVVDVSHEDPEVKFNWYVDGVEVHNAKTKPREEQYNSTYRVVSVLTV
LHQDWLNGKEYKCQVSNKALPAPIEKTISKAKGQPREPQVYTLPPSRDELTKNQVSLTCLVKG
FYPSDIAVEWESNGQPENNYKTTPPVLDSDGSFFLYSKLTVDKSRWQQGNVFSCSVMHEALHN
HYTQKSLSLSPG
Heavy chain constant region-IgG1(AQQ + YTE + K447 Del)
SEQ ID NO: 38, X1 = Y, X2 = T, X3 = E
ASTKGPSVFPLAPSSKSTSGGTAALGCLVKDYFPEPVTVSWNSGALTSGVHTFPAVLQSSGLYS
LSSVVTVPSSSLGTQTYICNVNHKPSNTKVDKKVEPKSCDKTHTCPPCPAPEAQGGPSVFLFPPK
PKDTLYITREPEVTCVVVDVSHEDPEVKFNWYVDGVEVHNAKTKPREEQYNSTYRVVSVLTV
LHQDWLNGKEYKCQVSNKALPAPIEKTISKAKGQPREPQVYTLPPSRDELTKNQVSLTCLVKG
FYPSDIAVEWESNGQPENNYKTTPPVLDSDGSFFLYSKLTVDKSRWQQGNVFSCSVMHEALHN
HYTQKSLSLSPG
Heavy chain of anti-CLDN18.2 antibody
QVQLVQSGAEVKKPGASVKVSCKASGYSFTNYWVNWVRQAPGQGLEWIGMVHPSGSETRLN
QQFKDKATLTVDKSTSTAYMELSSLRSEDTAVYYCARVWSGNAFDYWGQGTLVTVSSASTKG
PSVFPLAPSSKSTSGGTAALGCLVKDYFPEPVTVSWNSGALTSGVHTFPAVLQSSGLYSLSSVVT
VPSSSLGTQTYICNVNHKPSNTKVDKKVEPKSCDKTHTCPPCPAPELLGGPSVFLFPPKPKDTLM
ISRTPEVTCVVVDVSHEDPEVKFNWYVDGVEVHNAKTKPREEQYNSTYRVVSVLTVLHQDWL
NGKEYKCKVSNKALPAPIEKTISKAKGQPREPQVYTLPPSRDELTKNQVSLTCLVKGFYPSDIA
VEWESNGQPENNYKTTPPVLDSDGSFFLYSKLTVDKSRWQQGNVFSCSVMHEALHNHYTQKS
LSLSPGK (SEQ ID NO: 39)
Light chain of anti-CLDN18.2 antibody
DIVMTQSPDSLAVSLGERATINCLSSQSLLASGNQRNYLTWYQQKPGQPPKLLIYWASTRDSGV
PDRFTGSGSGTDFTLTISSVQAEDVAVYYCQNGFSFPFTFGQGTKLEIKRTVAAPSVFIFPPSDEQ
LKSGTASVVCLLNNFYPREAKVQWKVDNALQSGNSQESVTEQDSKDSTYSLSSTLTLSKADYE
KHKVYACEVTHQGLSSPVTKSFNRGEC (SEQ ID NO: 40)

Having thus described in detail preferred embodiments of the present invention, it is to be understood that the invention defined by the above paragraphs is not to be limited to particular details set forth in the above description as many apparent variations thereof are possible without departing from the spirit or scope of the present invention.

1. An isolated monoclonal antibody, or an antigen-binding portion thereof, capable of binding to Signal-regulatory protein alpha (SIRPα), comprising (i) a heavy chain variable region comprising a VH CDR1 region, a VH CDR2 region and a VH CDR3 region, and (ii) a light chain variable region comprising a VL CDR1 region, a VL CDR2 region and a VL CDR3 region,
wherein the VH CDR1 region, the VH CDR2 region, the VH CDR3 region, the VL CDR1 region, the VL CDR2 region and the VL CDR3 region comprise the amino acid sequences of
(1) SEQ ID NOs: 1, 2, 3, 4, 5 and 6, respectively,
wherein X1 and X2 in SEQ ID NO: 2 are N and A, respectively, and X in SEQ ID NO: 5 is A;
wherein X1 and X2 in SEQ ID NO: 2 are N and G, respectively, and X in SEQ ID NO: 5 is N; or
wherein X1 and X2 in SEQ ID NO: 2 are A and G, respectively, and X in SEQ ID NO: 5 is A; or
(2) SEQ ID NOs: 9, 10, 11, 12, 13 and 14, respectively,
wherein X in SEQ ID NO: 9 is Y, and X1 and X2 in SEQ ID NO: 10 are N and A respectively;
wherein X in SEQ ID NO: 9 is Y, and X1 and X2 in SEQ ID NO: 10 are N and G, respectively;
wherein X in SEQ ID NO: 9 is Y, and X1 and X2 in SEQ ID NO: 10 are A and G, respectively; or
wherein X in SEQ ID NO: 9 is F, and X1 and X2 in SEQ ID NO: 10 are N and G respectively.

2. (canceled)

3. The isolated monoclonal antibody, or the antigen-binding portion thereof, of claim 1, wherein the heavy chain variable region comprises an amino acid sequence having at least 85%, 86%, 87%, 88%, 89%, 90%, 91%, 92%, 93%, 94%, 95%, 96%, 97%, 98%, 99% or 100% identity to SEQ ID NOs: 7, 15 or 17,
wherein X1 and X2 in SEQ ID NO: 7 are N and A, respectively; N and G, respectively; or A and G, respectively;
wherein X1 and X2 in SEQ ID NO: 15 are N and A, respectively; N and G, respectively; or A and G, respectively.

4. The isolated monoclonal antibody, or the antigen-binding portion thereof, of claim 1, wherein the light chain variable region comprises an amino acid sequence having at least 85%, 86%, 87%, 88%, 89%, 90%, 91%, 92%, 93%, 94%, 95%, 96%, 97%, 98%, 99% or 100% identity to SEQ ID NOs: 8 or 18,
wherein X in SEQ ID NO: 8 is A or N;
wherein X in SEQ ID NO: 16 is S or N.

5. The isolated monoclonal antibody, or the antigen-binding portion thereof, of claim 2, wherein the heavy chain variable region and the light chain variable region comprise amino acid sequences having at least 85%, 86%, 87%, 88%, 89%, 90%, 91%, 92%, 93%, 94%, 95%, 96%, 97%, 98%, 99% or 100% identity to
(1) SEQ ID NOs: 7 and 8, respectively,
wherein X1 and X2 in SEQ ID NO: 7 are N and A, respectively, and X in SEQ ID NO: 8 is A;
wherein X1 and X2 in SEQ ID NO: 7 are N and G, respectively, and X in SEQ ID NO: 8 is N; or
wherein X1 and X2 in SEQ ID NO: 7 are A and G, respectively, and X in SEQ ID NO: 8 is A;
(2) SEQ ID NOs: 15 and 16, respectively,
wherein X1 and X2 in SEQ ID NO: 15 are N and A, respectively, and X in SEQ ID NO: 16 is S;
wherein X1 and X2 in SEQ ID NO: 15 are N and G, respectively, and X in SEQ ID NO: 16 is N; or
wherein X1 and X2 in SEQ ID NO: 15 are A and G, respectively, and X in SEQ ID NO: 16 is S; or
(3) SEQ ID NOs: 17 and 18, respectively.

6. The isolated monoclonal antibody, or the antigen-binding portion thereof, of claim 1, which is of IgG1, IgG2 or IgG4 isotype.

7. The isolated monoclonal antibody, or an antigen-binding portion thereof, of claim 1, comprising a heavy chain constant region comprising an amino acid sequence of SEQ ID NO: 19 or 38, linked to the heavy chain variable region, and a light chain constant region comprising an amino acid sequence of SEQ ID NO: 20, linked to the light chain variable region, wherein X1, X2 and X3 in SEQ ID NO: 38 are M, S and T, respectively; or Y, T and E, respectively.

8. The isolated monoclonal antibody, or the antigen-binding portion thereof, of claim 1, which (a) is able to bind human SIRPα; (b) is able to bind monkey SIRPα; (c) does not bind mouse SIRPα; (d) is able to bind human SIRPβ; (e) does not bind human SIRPγ; (f) is able to block SIRPα binding to CD47; (g) is able to be internalized by SIRPα-expressing cells; (h) is able to induce phagocytosis of tumor cells by macrophages, and/or (i) has in vivo anti-tumor activity

9. The isolated monoclonal antibody, or the antigen-binding portion thereof, of claim 1, which is chimeric or human.

10. A nucleic acid molecule encoding the isolated monoclonal antibody or the antigen-binding portion thereof of claim 1.

11. An expression vector comprising the nucleic acid molecule of claim 10.

12. A host cell comprising the expression vector of claim 11.

13. A pharmaceutical composition comprising the isolated monoclonal antibody, or antigen-binding portion thereof, of claim 1, and a pharmaceutically acceptable carrier.

14. A method for treating a cancer associated with SIRPα in a subject in need thereof, comprising administering to the subject a therapeutically effective amount of the pharmaceutical composition of claim 13.

15. The method of claim 14, wherein the cancer is a solid cancer or a hematologic cancer.

16. The method of claim 15, wherein the cancer is non-small lung cancer, breast cancer, ovarian cancer, renal cell cancer, colorectal cancer, or pancreatic cancer.

17. The method of claim 14, wherein the subject is further administered with an antibody targeting a tumor associated antigen.

18. The method of claim 17, wherein the tumor associated antigen is selected from the group consisting of CD19, CD20, CD22, CD4, CD24, CD38, CD123, CD228, CD138, BCMA, GPC3, CEA, folate receptor (FRα), mesothelin, CD276, gp100, 5T4, GD2, EGFR, MUC-1, PSMA, EpCAM, MCSP, SM5-1, MICA, MICB, ULBP and HER-2.

19. The method of claim 14, wherein the subject is further administered with an antibody targeting an inhibitory immune checkpoint.

20. A method for enhancing an immune response in a subject in need thereof, comprising administering to the subject the pharmaceutical composition of claim 13.
